SEQUENCE LISTING (29 sequences)

SEQ ID NO: 1 - protein, 414 aa, Arthrobacter sp.
  misc_feature 1..414: Arthrobacter sp. DSM 3747 (strain)
  HyuC
MTLQKAQAERIEKEIWELSRFSAEGPGVTRLTYTPEHAAARETLIAAMEAAALSVREDALGNIIGRREGTDPQLPAIAVGSHFDSVRNGGMFDGTAGVVCALEAARVMLESGYVNRHPFEFIAIVEEEGARFSSGMLGGRAIAGLVADRELDSLVDEDGVSVRQAATAFGLKPGELQAAARSAADLRAFIELHIEQGPILEQEQIEIGVVTSIVGVRALRVAVKGRSAHAGTTPMHLRQDALVPAALMVREVNRFVNEIADGTVATVGHLTVAPGGGNQVPGEVEFTLDLRSPHEESLRVLINRISVMVGEVASQAGVAADVDEFFNLSPVQLAPTMVDAVREAASALQFTHRDISSGAGHDSMFIAQVTDVGMVFVPSRAGRSHVPEEWTDFDDLRKGTEVVLRVMKALDRGS

SEQ ID NO: 2 - protein, 409 aa, Geobacillus stearothermophilus
  AmaB
MIQGERLWQRLMELGEVGKQPSGGVTRLSFTAEERRAKDLVASYMREAGLFVYEDAAGNLIGRKEGTNPDATVVLVGSHLDSVYNGGCFDGPLGVLAGVEVVQTMNEHGVVTHHPIEVVAFTDEEGARFRFGMIGSRAMAGTLPPEALECRDAEGISLAEAMKQAGLDPDRLPQAARKPGTVKAYVELHIEQGRVLEETGLPVGIVTGIAGLIWVKFTIEGKAEHAGATPMSLRRDPMAAAAQIIIVIEEEARRTGTTVGTVGQLHVYPGGINVIPERVEFVLDLRDLKAEVRDQVWKAIAVRAETIAKERNVRVTTERLQEMPPVLCSDEVKRAAEAACQKLGYPSFWLPSGAAHDSVQLAPICPIGMIFVRSQDGVSHSPAEWSTKEDCAAGAEVLYHTVWQLAQGE

SEQ ID NO: 3 - protein, 420 aa, Pseudomonas sp.
  misc_feature 1..420: Pseudomonas sp. QR-101 (strain)
  AtcC
MSGVNSMNRTPGIQPERLWGDLMSTAKFGALGETGMRRLALSNEDRQVRDWFVAQCRELGCTIDIDQIGNIFATYPGLDASLAPIAMGSHLDTQPAGGRFDGILGVLAGIEVLRSLHDASVRPAHPITVIVWTNEEGSRFAPAMMGSGVYCGAHQWETVAATCDKSGVSVAQALDAIGYAGEHEPGFMQFSAYLELHIEQGPVLEAENIEIGVVDAVQGVCWLDIKVPGVSAHAGGRPMTMRDDALVAASKIVLAVEAVASAHLPGVGTIGYISAGPNSRNVIPGTVALEVDLRHPDDAELSALENEVTAEIKRICPAAEVQRVWRKPPVTFDSRIVDAIAARTEALGYSARRMVSGAGHDAAHVAGIAPSAMIFIPSYLGLSHNVREYSSPEQCAQGATVLLGAVLDLDNLLLADSRTG

SEQ ID NO: 4 - protein, 409 aa, Geobacillus kaustophilus
  Lnc
MIQGERLWQRLMELGEVGKKPSGGVTRLSFTAEERRAKDLVASYMREAGLFVYEDTAGNLIGRKEGANPDAPVVLVGSHLDSVYNGGCFDGPLGVLAGVEVVQTMNEHGVVTHHPIEVVAFTDEEGARFRFGMIGSRAMAGTLLPEALECRDANGISIAEAMRQTGLDPDRLPQAARKPGTVKAYVELHIEQGRVLEEAGLPVGIVTGIAGLIWVKFIIEGKAEHAGATPMSLRRDPMAAAAQIITVIEEEARRTGTTVGTVGQLHVYPGGINVIPERVEFVLDLRDLKAEVRDQVWNDIASRAETIAKERNVRLTTERLQEMAPVLCSEVVKQAAERACKQLGYPPFWLPSGAAHDGVQLAPICPIGMIFVRSQDGVSHSPAEWSTKEDCAVGAEVLYHTVWQLAQGE

SEQ ID NO: 5 - protein, 412 aa, Paenarthrobacter aurescens
  HyuC
MTLQKAQAERIEKEIRELSRFSAEGPGVTRLTYTPEHAAARETLIAAMKAAALSVREDALGNIIGRREGTDPELPAIAVGSHFDSVRNGGMFDGTAGVVCALEAARVMLENGYVNRHPFEFIAIVEEEGARFSSGMLGGRAIAGLVADRELDSLVDEDGVSVRQAATAFGLKPGELQAAARSAADLRAFIELHIEQGPILEQEQIEIGVVTSIVGVRALRVAVKGRSDHAGTTPMHLRQDALVPAALMVREVNRFVNEIADGTVATVGHLTVAPGGGNQVPGEVDFTLDLRSPHEESLRVLIDRISVMVGEVASQAGVAADVDEFFNLSPVQLAPTMVDAVREAASALQFTHRDISSGAGHDSMFIAQVTDVGMVFVPSRAGRSHVPEEWTDFDDLRKGTEVVLRVMKALDR

SEQ ID NO: 6 - protein, 416 aa, Sinorhizobium meliloti
  SinmeB_2280
MAAPGENRRVNADRLWDSLMEMAKIGPGVAGGNNRQTLTDADGEGRRLFQSWCEEAGLSMGVDKMGTMFLTRPGTDPDALPVHIGSHLDTQPTGGKFDGVLGVLSGLEAVRTMNDLGIKTKHPIVVTNWTNEEGARFAPAMLASGVFAGVHTLEYAYARKDPEGKSFGDELKRIGWLGDEEVGARKMHAYFEYHIEQGPILEAENKQIGVVTHCQGLWWLEFTLTGREAHTGSTPMDMRVNAGLAMARILEMVQTVAMENQPGAVGGVGQMFFSPNSRNVLPGKVVFTVDIRSPDQAKLDGMRARIEAEAPKICERLGVGCSIEAVGHFDPVTFDPKLVETVRGAAEKLGYSHMNLVSGAGHDACWAAKVAPTTMIMCPCVGGLSHNEAEDISREWAAAGADVLFHAVLETAEIVE

SEQ ID NO: 7 - protein, 413 aa, Bacillus fordii
  misc_feature 1..413: Bacillus fordii MH602 (strain)
MEKQKVLINGERLKDTIEEFADFGRTEKNGVTRLALSDVDVKARRHFQSLCEQLGMSVVWDDMGNMYAKLPGIDNDQPPVVIGSHLDSVKKGGRFDGTLGVLTGLEVVRTMVENGIKPEIPIIVANITNEEGARFEPSLMASGVLSGRFDKAAMLKSTDVDGITFAEALKKSGYEGKKENRLKEAAAFLELHIEQGPVLESEDIQIGIVECVVGMVCFEIEVTGESDHAGTTPMSMRKDALFAANQLISEIRQKMNRLDDQLVYTVGRMTVSPNIHTVIPNKVVFTIGARHQDGKIIRQVEEIIQGLPNSSGKEKCNVTTTKLWDRHTVWFNEEIVNTLEKSARSLGYSFKRMVSGAGHDAQFIATYIPTAMVFVPSINGKSHDEDELTTWEDCENGVNVILQTVLDLTTDKL

SEQ ID NO: 8 - protein, 412 aa, Arthrobacter sp.
  misc_feature 1..412: Arthrobacter sp. BT801 (strain)
  HyuC
MTSLRAQADRIEKDIRDLARFSAGGPGVTRLSYTPEHAAARDLIIAAMQQAGLDVREDGLGNITGRREGSDPDLPAIAVGSHFDSVRNGGMFDGTAGVVCALEAARVVNESEYVNRHPFEFIAIVEEEGSRFNSGMLGGRALAGLVTDEDLDKLKDEDGISVRDAATEFGLQPGNLQTSVRSRLDLRAFIEPHIEQGPVLEQEGIEIGVVTSIVGIRTLRVAVLGRSDHAGTTPMHLRQDALIPSALMVREVNRLVNELGDETVATVGHLTVAPGGINQVPGEVNFTLDLRSPHEESLKQIVKQITVMIQEVASQAEVTANVEEFFSLDPVPLAPSVVDAVRQAASNLRFSHRDMPSGAGHDSMFISQVTDVGMVFVPSRDGRSHVPEEWSDFDDLRKGTDVVLGVMTALDK

SEQ ID NO: 9 - protein, 412 aa, Microbacterium liquefaciens
  misc_feature 1..412: Microbacterium liquefaciens AJ 3912 (strain)
MTLQQARADRIEEELWTLSRFSVEGPGVTRLTYTPEHAAAREVIVAAMQRTGLSVHEDALGNIIGRREGSDPALPAIAFGSHFDSVRNGGMFDGTAGVVCALEAARVLQESGYVNRHPLEVIAIVEEEGTRFSSGMLGGRAIAGLVSDADLDTLVDEDGVTVREAATAFGLEPGELRTAARTRDDLRAFIELHIEQGPILEQEKVEIGVVTGIVGVRAFRITVEGRSDHAGTTPMHLRQDALVPAALMVREINRFVNEIADGTVATVGHLTVTPGGLNQVPGGVEFTLDLRSPHEESIRLLVDRIEAMVAEVAAAAGVEAAVNGFFALSPVGLSPVVVDRVRDAASELGFTHRDITSGAGHDSMFIAQITDVGMVFVPSRAGRSHVPEEWSDFDDLRKGTDVVLHVVTALDR

SEQ ID NO: 10 - protein, 420 aa, Arthrobacter sp.
  misc_feature 1..420: Arthrobacter sp. DSM 9771 (strain)
  HyuH
MFDVIVKNCRMVSSDGITEADILVKDGKVAAISSDTSDVEASRTIDAGGKFVMPGVVDEHVHIIDMDLKNRYGRFELDSESAAVGGITTIFEMPFTFPPTTTLDAFLEKKKQAGQRLKVDFALYGGGVPGNLPEIRKMHDAGAVGFKSMMAASVPGMFDAVSDGELFEIFQEIAACGSVAVVHAENETIIQALQKQIKAAGRKDMAAYEASQPVFQENEAIQRALLLQKEAGCRLIVLHVSNPDGVELIHRAQSEGQDVHCESGPQYLNITTDDAERIGPYMKVAPPVRSAEMNVRLWEQLENGLIDTLGSDHGGHPVEDKEPGWKDVWKAGNGALGLETSLPMMLTNGVNKGRLSLERLVEVMCEKPAKLFGIYPQKGTLQVGSDADLLILDLDIDTKVDASQFRSLHKYSPFDGMPVT

SEQ ID NO: 11 - protein, 479 aa, Pseudomonas fluorescens
MSLLIRGATVITYDERYRADVLCAGGLIRAIGTGLDVPAGTEVLDGSGQYLMPGGIDPHTHMQLPFMGTVASEDFFSGTAAGLAGGTTSIIDFVIPNPQQSLMEAFHQWRGWAEKSASDYGFHVAITWWSEQVREEMAELVSHHGINSFKHFMAYKNAIMAADDTLVASFERCLELGAVPTVHAENGELVYHLQRKLMAQGITGPEAHPLSRPSQVEGEAASRAIRIAETIGTPLYLVHVSTKEALDEITYARGKGQPVYGEVLAGHLLLDDSVYQHPDWHTAAGYVMSPPFRPRGHQEALWHGLQSGNLHTTATDHCCFCAEQKAAGRDDFSKIPNGTAGIEDRMALLWHEGVNTGRLSMQEFVALTSTNTAKIFNLYPRKGAIRVGADADLVLWDPEGTRTISAKTHHQKVDFNIFEGKTVRGVPSHTISQGKLVWADGDLRAERGAGRYVERPAYPSVFEQLSKRAEHSRPTAVKR

SEQ ID NO: 12 - protein, 479 aa, Pseudomonas aeruginosa
  Dht
MSLLIRGATVVTHEESYRADVLCANGLIQAIGENLETPSGCDVLDGGGQYLMPGGIDPHTHMQLPFMGTVASEDFFSGTAAGLAGGTTSIIDFVIPNPRQSLLEAFHTWRGWAQKSAADYGFHVAITWWSDEVAREMGELVAQHGVNSFKHFMAYKNAIMAADDTLVASFERCLELGAVPTVHAENGELVFHLQQKLLAQGLTGPEAHPLSRPPQVEGEAASRAIRIAETLGTPLYLVHISSREALDEIAYARAKGQPVYGEVLAGHLLLDDSVYRHPDWATAAGYVMSPPFRPVEHQEALWRGLQSGNLHTTATDHCCFCAEQKAMGRDDFSKIPNGTAGIEDRMALLWDAGVNSGRLSMHEFVALTSTNTAKIFNLFPRKGAIRVGADADLVLWDPQGSRTLSAATHHQRVDFNIFEGRTVRGIPSHTISQGKLLWAAGDLRAEPGAGRYVERPAYPSVYEVLGRRAERQRPVAVER

SEQ ID NO: 13 - protein, 475 aa, Bacillus fordii
  misc_feature 1..475: Bacillus fordii MH602 (strain)
MKKIIKDGTVVTSTDVYTADVLIEDEKIQAIGQNISDIYAEVIDASGYYVMPGGIDPHTHMDMPFGGTVTADDFETGSIAAACGGTTTIIDFCLTGKGKSLKSALEKWHAKSAGKAVIDYGFHLQIAEANEEVFMEMPQIIEEEGVTSFKIFMAYKDVLQADDETLFQTLVTAREHGGLVMVHAENGDVINYLTEKALKEGNTEPIYHALTRPPELEGEATGRAARLTALADSQLYVVHVTCKEAVEQIAEARRRGYRVFGETCPQYLVLDQTYLERPDFEGAKYVWSPPLRERSNQEVLWNALKSGDLQAIGSDHCSFNFRGQKELGKNDFSKIPNGGPFVEDRFSVLFSEGVKKGRISIHDFVNIISTQAAKLFGLFPRKGTIAPGSDADIVIFDPNVERIISVETHHMNVDYSALEGLKIIGEPITVLSRGNYVVKDKEFVGKPGKGKFLKCNKFNHDLYKETGQLEGLLKG

SEQ ID NO: 14 - protein, 458 aa, Arthrobacter sp.
  misc_feature 1..458: Arthrobacter sp. BT801 (strain)
  HyuH
MFDVIVKNCRIVSSEGITEADILVKEGKVAAISSEGLNDEVQETIDAGGKFVLPGVVDEHVHIIDMDLKDQNGRFEFDSESAAVGGITTIIEMPITSPPTTTLEAFLAKKKEAGDRLKVDFALYGGGVPGNLSEIRKMHEAGAVGFKSMMAASVPGMFEAVTDGELFEIFQEIATCGSVIVVHAENETIIQSLQRQLKEAGRTDMAAYEASQPAFQENEAIQRALALQKETGCRLIVLHVSNPDGVQLIHQAQSSGQDVHCESGPQYLNITAEDASRIGPYMKIAPPVRSAEMNARLWEQLEKGLIDTLGSDHGGHPKENKERGWDNVWDASNGALGLETSLPMMLTNGVNRGRLSLERLVEVMCENPAKLFGIYPQKGTLQVGSDADLVIVDLEVDDKVDASEFRSLLKHSPFDGMPVTGKPVLTMVRGTVVAENGKILVEPGFGQFVTCRESQDPK

SEQ ID NO: 15 - protein, 455 aa, Alcaligenes faecalis
  misc_feature 1..455: Alcaligenes faecalis subsp. faecalis (subspecies)
MSDFDLVVRGNIVERDRIITDGWVAVREGRVAARGIGAAPSAREHVDARGMWVMPGVLDGQVHSGSQANQEGLGWASRAAAAGGVTTMVEMPHDDPEPVASRAQLEDKIARIESDCHVDVACYGTLNDQHGLEAAAGLIEGGVCGFKFSTFEASPNRSPRVEEDILMEAFRLIAPSGLACGVHNQDQELTRKNIKRMVEAGDTGWDAFLRAHTPLIENLATSAIYEIGAQTGARAHAVHVSLSRGFEICNMYRRAGYKASIETCVQYLMLNHEEHTRRFGAKTKHYPPIRPKAEMDLLWTHIANDECTFVSSDHVSWGLERKQFENVFQNSSGGPGLETLLPAFWTGCAEHGISPTMVVKQLCWGPAQHFLLEHRKGSLNVGADADIVIVKPDTYRFDPSTSLSAVTWSSFEDRELQVRVEATYVRGQLAWDGKTIRNAAGDGQFIRPHKAGELS

SEQ ID NO: 16 - protein, 459 aa, Microbacterium liquefaciens
  misc_feature 1..459: Microbacterium liquefaciens AJ 3912 (strain)
MFDVIVKNCRVVSSQGIIEADILVKDGRIAAISEEPLEAEAARTIDAAGRFVMPGVVDEHVHIIDMDLKEVYGRFELDSESAAVGGVTTIIEMPITFPPTTTLEAFLEKKKQGEQRLKVDFALYGGGVPGNLSEIRKMHDAGAVGFKSMMAASVPGMFEAVDDGQLFEIFQEIAACGSVIVVHAENEMLIQTLQKQLKAAGRKDLAAYEASQPVFQENEAIQRALLLQKEAGCRLIVVHVSNPGGVELIHKAQSEGQDVHCESGPQYLNLTMDDAEKVGPYMKIAPPVRSAELNAVLWEQLEKGYIDTLGSDHGGHPVENKEGGWDDIWTASNGALGLETSLPMMLTNGVNKGRVSLERLVEVMCENPAKLFGIYPQKGTLQVGSDADLLILDLEIEDRKVDASQFRSLLHYSPFDGRPVTGAPVLTMIRGTVVAQDGEILVDQGFGQFVTRRDSEVSS

SEQ ID NO: 17 - protein, 236 aa, Arthrobacter aurescens
  misc_feature 1..236: Arthrobacter aurescens DSM 3745 (strain)
  HyuR
MRILVINPNSSSALTESVADAAQQVVATGTIISAINPSRGPAVIEGSFDEALATFHLIEEVERAERENPPDAYVIACFGDPGLDAVKELTDRPVVGVAEAAIHMSSFVAATFSIVSILPRVRKHLHELVRQAGATNRLASIKLPNLGVMAFHEDEHAALETLKQAAKEAVQEDGAESIVLGCAGMVGFARQLSDELGVPVIDPVEAACRVAESLVALGYQTSKANSYQKPTEKQYL

SEQ ID NO: 18 - protein, 249 aa, Pseudomonas sp.
  misc_feature 1..249: Pseudomonas sp. NS671 (strain)
  HyuE
MKIKVINPNTTLAMTKGIEHAAKSAARSDTQIVAVSPKMGPASIESYYDEYLSIPGVIEEIKKGEEEGVDAFVIACWGDPGLHAAREVTDKPVVGIAESSVYLASMLAARFSVVTVLPRIKTMLEDLVDSYGMQKRVLNIRTTPMGVLDFERDPEAGIEMLRQEGKRAVEEDNAEAILLGCAGMAEFADSLEKELGVPVIDGVVAGVKFAETIVDLGKKTSKLKTYKYPEKKEYVGALENFGRNQTTTK

SEQ ID NO: 19 - protein, 244 aa, Rhodococcus
  misc_feature 1..244: Rhodococcus R04 (strain)
  Hyu2
MFIKVVNPNTTWSMTATIEACARAVAGPGTRVEAVSPTMGPPSIESHYDDALAVPGILTEIEKGERDGVDGYVIACFGDPGLDAARELAGGPVVGIAEAAMHTAAVLGRGFSVVTTLARTTGRAWDLAHRYGMRDACRGVHACDLPVLALDSEPDARKIVTEACLDALYEDGSDAIVLGCAGMADLCAHISAEIGVPVVDGVAAATLTVQSLVTMGLATGKRGEFAAPPPKRYAGLLDGFTTGG

SEQ ID NO: 20 - protein, 236 aa, Microbacterium liquefaciens
  misc_feature 1..236: Microbacterium liquefaciens AJ 3912 (strain)
  HRase
MRIHVINPNSSVDLTDAVAEAARSVVSPGTTITAVNPSKGPTVIEGSYDEVLATYHLVEEVRRAEREDRPDAYVIACFGDPGLDAVRELTDRPVVGIAEAAIQMTSFVAASFSIVSILPRVRKHLHELVHRAGATDRLASLKLPDLGVLAFHEDEAAAFETLRRVAGEAVREDGAESIVLGCAGMAGFARQLSEELGVPVIDAVEAACRVAESLVALGYRTSKANTYQAPTEKQYL

SEQ ID NO: 21 - protein, 241 aa, Sinorhizobium meliloti
  misc_feature 1..241: Sinorhizobium meliloti strain CECT 4114 (strain)
  HyuA
MHIHLINPNSTASMTAQALESALLVKHAHTHVSASNPTDTPASIEGGADEAMSVPGMLAEIRQGEAQGVDAYVIACFDDPGLHAAREVAKGPVIGICQAAVQVAMTISRRFSVITTLPRSVPIIEDLVSDYGAERHCRKVRAIDLPVLALEEDPQRAERLLLKEIEIAKAEDGAEAIVLGCAGMSSLCDRLQKATGVPVIDGVTAAVKMAEALLGAGYATSKVNTYAYPRIKAAAGHKVCA

SEQ ID NO: 22 - protein, 243 aa, Flavobacterium sp.
  misc_feature 1..243: Flavobacterium sp. AJ 11199 (strain)
  HRase
MKIKVINPNTTLTMTAKIGEAAAAVASAGTEVVAVSPAMGPASIEGHYDEAVSALGVLDEVRKGKAEGCDGYLIACFDDPGLQAAREIADGPVVGIAEAAMHMASFVSEGFSVVATGHRSRIILEHLARSYGMEHKCRKVRTTELAVLDLEVEGSDARGIILEECRRAIVEDHSDCIVLGCAGMADLADYISKELGVPVVDGVAAGVKVLEGLIGLRLSTSRACGYAYPNPKTYSGEMARFQP

SEQ ID NO: 23 - protein, 247 aa, Agrobacterium sp.
  misc_feature 1..247: Agrobacterium sp. IP I-671 (strain)
  HyuA
MKIKVINPNTTWTMTDKIAGAARAAAAPGTEIVAVSPDMGPVSIEGYYDEVFAAVGVVDEVRKGELEGCDGYVIACFGDPGLNAAREVARGPVIGIAEAAMHAASLIGGSFSIISMLGRSRGVLEHLVHSYGMAHKCRSVRMTDLPVLEFEEEGSDARRIVVEECRRAIEQDHADSVLLGCGGMSDLMAYVSQEIGAPAIDGVSAGVKLVEALVGMGLGTSKRAGLRLSDRESLHCSFSSFAPSRPR

SEQ ID NO: 24 - protein, 247 aa, Pasteurella sp.
  misc_feature 1..247: Pasteurella sp. AJ 11221 (strain)
MKIKVINPNTTWSMTEKIGEAARRVAAPGTEIVAVSPAMGPVSIEGFYDEAFAAIGVIDEVRKGEEEGCDGYVIACFGDPGLLAAREIARGPVVIAEAAMHAASLIGNGFTIVSMLERTRATMEHLVHAYGMSHKCRNIRMTDLPVLELEKEGSNAQAIIMEECRRALEEDHSDAVLLGCGGMSDLMALITREIGAPAIDGVSSGVKLVEALVSLGLGTSKRQAYAYPVEKTYTGSFSQFSVPAANV

SEQ ID NO: 25 - protein, 228 aa, Pyrococcus horikoshii
  misc_feature 1..228: Pyrococcus horikoshii OT3 (strain)
  PH1054
MYRMDKYTIGLIRVITLEDKEILNLHGRIIESAFPELKVVSRCIEDQPKGIYNEETEREAEPKIIRLAKEFEREGVDAIIISCAADPAVEKVRKLLSIPVIGAGSSVSALALAYGRRVGVLNLTEETPKVIRSILGNNLIAEDHPSGVSNTLDLLTDWGRREVINAAKRLKEKGVEVIALGCTGMSTIGIAPVLEEEVGIPVIDPVIASGAVALHALKRREVKRFEGR

SEQ ID NO: 26 - protein, 236 aa, Arthrobacter sp.
  misc_feature 1..236: Arthrobacter sp. BT801 (strain)
  HyuA
MRIHIINPNSSSELTEAVAETARSVVSAGTAVTAVNPSSGPAVIEGSYDEALATYHLVQEVMRAEREDRPDAYVIACFGDPGLNAVRELTEKPVVGIAEAAIQLSSFIGATFSIVSTLPRVRSHLHGLVRRAGATNRLASVKTPDLGVMAFHEDAGSAQATLEQAAKEAVQEDGAEVVVLGCAGMAGLARRLSEELQVPVIDPVEAACRVAESLSALGYGTSKANTYEKPTEKVYN

SEQ ID NO: 27 - DNA, 1263 bp, Arthrobacter aurescens
  misc_feature 1..1263: Arthrobacter aurescens DSM 3747 (strain)
  hyuC
atgaccctgcagaaagcgcaagcggagcgcattgagaaagagatctgggagctctcccggttctcggcggaaggccccggtgttacccggctgacctacactccagagcatgccgccgcgcgggaaacgctcattgcggctatggaagcggccgctttgagcgttcgtgaagacgctctcgggaacatcatcggccgacgtgaaggcactgatccgcagctccctgcgatcgcggtcggttcacacttcgattctgtccgaaacggcgggatgttcgatggcactgcaggcgtggtgtgcgcccttgaggctgcccgggtgatgctggagagcggctacgtgaatcggcatccatttgagttcatcgcgatcgtggaggaggaaggggcccgcttcagcagtggcatgttgggcggccgggccattgcaggtttggtcgccgacagggaactggactctttggttgatgaggatggagtgtccgttaggcaggcggctactgccttcggcttgaagccgggcgaactgcaggctgcagcccgctccgcggcggacctgcgtgcttttatcgaactacacattgaacaaggaccgatcctcgagcaggagcaaatagagatcggagttgtgacctccatcgttggcgttcgcgcattgcgggttgctgtcaaaggcagaagcgcacacgccggcacaacccccatgcacctgcgccaggatgcgctggtacccgccgctctcatggtgcgggaggtcaaccggttcgtcaacgagatcgccgatggcacagtggctaccgttggccacctcacagtggcccccggtggcggcaaccaggtcccgggggaggtggagttcacactggacctgcgttctccgcatgaggagtcgctccgggtgttgatcaaccgcatctcggtcatggtcggcgaggtcgcctcgcaggccggtgtggctgccgatgtggatgaatttttcaatctcagcccggtgcagctggctcctaccatggtggacgccgttcgcgaagcggcctcggccctgcagttcacgcaccgggatatcagcagtggggcgggccacgactcgatgttcatcgcccaggtcacggacgtcggaatggttttcgttccaagccgtgctggccggagccacgttcccgaagaatggaccgatttcgatgaccttcgcaagggaactgaggttgtcctccgggtaatgaaggcacttgaccggggatcccatcatcatcatcatcattga

SEQ ID NO: 28 - DNA, 1377 bp, Arthrobacter sp.
  misc_feature 1..1377: Arthrobacter sp. DSM 9771 (strain)
  hyuH
atgtttgacgtaatagttaagaactgccgtatggtgtccagcgacggaatcaccgaggcagacattctggtgaaagacggcaaagtcgccgcaatcagctcggacacaagtgatgttgaggcgagccgaaccattgacgcgggtggcaagttcgtgatgccgggcgtggtcgatgaacatgtgcatatcatcgacatggatctgaagaaccggtatggccgcttcgaactcgattccgagtctgcggccgtgggaggcatcaccaccatctttgagatgccgtttaccttcccgcccaccaccactttggacgccttcctcgaaaagaagaagcaggcggggcagcggttgaaagttgacttcgcgctctatggcggtggagtgccgggaaacctgcccgagatccgcaaaatgcacgacgccggcgcagtgggcttcaagtcaatgatggcagcctcagttccgggcatgttcgacgccgtcagcgacggcgaactgttcgaaatcttccaggagatcgcagcctgtggttcagtcgccgtggtccatgccgagaatgaaacgatcattcaagcgctccagaagcagatcaaagccgctggtcgcaaggacatggccgcctacgaggcatcccaaccagttttccaggagaacgaggccattcagcgtgcgttactactgcagaaagaagccggctgtcgactgattgtgcttcacgtgagcaaccctgacggggtcgagctgatacatcgggcgcaatccgagggccaggacgtccactgcgagtcgggtccgcagtatctgaatatcaccacggacgacgccgaacgaatcggaccgtatatgaaggtcgcgccgcccgtccgctcagccgagatgaacgtcagattatgggaacaacttgagaacgggctcatcgacacccttgggtcagaccacggcggacatcctgtcgaggacaaagaacccggctggaaggacgtgtggaaagccggcaacggtgcgctgggccttgagacatccctgcctatgatgctgaccaacggagtgaataaaggcaggctatccttggaacgcctcgtcgaggtgatgtgcgagaaacctgcgaagctctttggcatctatccgcagaagggcacgctacaggttggttccgacgccgatctgctcatcctcgatctggatattgacaccaaagtggatgcctcgcagttccgatccctgcataagtacagcccgttcgacgggatgcccgtcacgggtgcaccggttctgacgatggtgcgcggaacggtggtggcagagaagggagaagttctggtcgagcagggattcggccagttcgtcacccgtcacgactacgaggcgtcgaagtga

SEQ ID NO: 29 - DNA, 711 bp, Arthrobacter aurescens
  misc_feature 1..711: Arthrobacter aurescens DSM 3745 (strain)
  hyuR
atgagaatcctcgtgatcaaccccaacagttccagcgcccttactgaatcggttgcggacgcagcacaacaagttgtcgcgaccggcaccataatttctgccatcaacccctccagaggacccgccgtcattgaaggcagctttgacgaagcactggccacgttccatctcattgaagaggtggagcgcgctgagcgggaaaacccgcccgacgcctacgtcatcgcatgtttcggggatccgggacttgacgcggtcaaggagctgactgacaggccagtggtaggagttgccgaagctgcaatccacatgtcttcattcgtcgcggccaccttctccattgtcagcatcctcccgagggtcaggaaacatctgcacgaactggtacggcaagcgggggcgacgaatcgcctcgcctccatcaagctcccaaatctgggggtgatggccttccatgaggacgaacatgccgcactggagacgctcaaacaagccgccaaggaggcggtccaggaggacggcgccgagtcgatagtgctcggatgcgccggcatggtggggtttgcgcgtcaactgagcgacgaactcggcgtccctgtcatcgaccccgtcgaggcagcttgccgcgtggccgagagtttggtcgctctgggctaccagaccagcaaagcgaactcgtatcaaaaaccgacagagaagcagtacctctag

(19) United States
(12) Patent Application Publication
(10) Pub. No.: US 2024/0287558 A1
(43) Pub. Date: Aug. 29, 2024

(54) ENZYMATIC METHOD FOR THE PRODUCTION OF L-GLUFOSINATE P-ALKYL ESTERS

(71) Applicant: Evonik Operations GmbH, Essen (DE)

(72) Inventors: Ludger Lautenschütz, Hanau (DE); Steffen Oßwald, Nidderau (DE); Markus Pötter, Muenster (DE); Jakob Müller, Haltern am See (DE)

(73) Assignee: Evonik Operations GmbH, Essen (DE)

(21) Appl. No.: 18/569,989

(22) PCT Filed: May 31, 2022
(86) PCT No.: PCT/EP2022/064682

§ 371 Date: Dec. 13, 2023

(30) Foreign Application Priority Data
Jun. 16, 2021 (EP) 21179773.3

(51) Int. Cl. C12P 13/04; C12N 9/80; C12N 9/90

(52) U.S. Cl. CPC C12P 13/04; C12N 9/80; C12N 9/90; C12Y 305/01087; C12Y 305/02002; C12Y 501/99005

(57) ABSTRACT

An enzymatic method for the production of an L-glufosinate P-alkyl ester can be performed. This method is characterized by reacting an L-glufosinate P-alkyl ester carbamoylate to give the corresponding L-glufosinate P-alkyl ester. This is catalyzed by a carbamoylase. The L-glufosinate P-alkyl ester carbamoylate employed may be obtained by a reaction from the corresponding L-glufosinate hydantoin P-alkyl ester. This is catalyzed by a hydantoinase, preferably an L-enantioselective hydantoinase. In a second aspect, an enzymatic method for enantioselective production of an L-glufosinate P-alkyl ester from a mixture M_{IIIA} of L- and D-glufosinate P-alkyl ester hydantoins can be performed. The L-glufosinate P-alkyl ester obtained in the methods according to the first or second aspect of the invention may be saponified to give L-glufosinate.

The present invention relates in a first aspect to an enzymatic method for the production of an L-glufosinate P-alkyl ester. This method is characterized by a step (c) in which an L-glufosinate P-alkyl ester carbamoylate is reacted to give the corresponding L-glufosinate P-alkyl ester. This step (c) is catalyzed by a carbamoylase, preferably by an L-enantioselective carbamoylase. The L-glufosinate P-alkyl ester carbamoylate employed in step (c) may be obtained by reaction from the corresponding L-glufosinate hydantoin P-alkyl ester. This optional, antecedent step (b) is catalyzed by a hydantoinase, preferably an L-enantioselective hydantoinase. Even more preferred, the L-glufosinate hydantoin P-alkyl ester employed in step (b) may be obtained by reaction from the corresponding D-glufosinate hydantoin P-alkyl ester. This optional, antecedent step (a) is preferably catalyzed by a hydantoin racemase.

In a second aspect, the present invention relates to an enzymatic method for enantioselective production of an L-glufosinate P-alkyl ester from a mixture M_{IIIA} of L- and D-glufosinate P-alkyl ester hydantoins. In the method according to the second aspect of the invention, the mixture M_{IIIA} is provided in a step (i-A) and employed in steps (ii) and (iii). Steps (ii) and (iii) correspond to steps (b) and (c) described above, respectively, wherein in step (c), an L-carbamoylase is employed. In an optional, preferred step (i-B) of the method according to the second aspect of the invention, corresponding to preferred step (a) described above, a hydantoin racemase is preferably used to convert at least a part of the D-glufosinate P-alkyl ester hydantoin in the mixture M_{IIIA} into its corresponding L-enantiomer, giving a mixture M_{IIIB}, which is then employed in steps (ii) and (iii). The L-glufosinate P-alkyl ester obtained in the methods according to the first or second aspect of the invention may be saponified to give L-glufosinate.

### 1. BACKGROUND OF THE INVENTION

Organic phosphorous compounds, i.e. chemical agents comprising a carbon-phosphor bond, are widely applied as herbicides in the area of plant protection. Agents such as the herbicides glyphosate (Roundup®, Touchdown®) and glufosinate (Basta®, Liberty®) as well as the growth regulator glyphosine (Polaris®) are used for this purpose (as described for example by G. Hörlein, Rev. Environ. Contam. Toxicol. 1994, 138, 73-145).

The esters of P-methyl phosphinic acid (for example, P-methyl phosphinic acid butyl ester; “MPBE”; CAS-No: 6172-80-1) have a key role as synthetic building blocks in the synthesis of the non-selective herbicide glufosinate. These esters are accessible via two fundamental synthetic pathways (summarized in FIGS. 3a and 3b, page 130, of the article of K. Haack, Chem. Unserer Zeit 2003, 37, 128-138):
a. Reacting diethyl chlorophosphite [CIP(OC₂H₅)₂] with CH₃MgCl provides methyl diethoxy phosphine [H₃CP(OC₂H₅)₂; “DEMP”; CAS-No. 15715-41-0], which is partially hydrolyzed to give the corresponding methylphosphinic acid ethyl ester (MPEE; CAS-Nr: 16391-07-4).
b. Alternatively, methane can be reacted with phosphor trichloride at 500° C. to give methyl dichloro phosphane H₃CPCl₂. The latter can then be solvolyzed in alcohols to give the corresponding methyl phosphinic acid esters.

The esters of P-methyl phosphinic acid add to carbon-carbon double bonds regioselectively. This property is used in the synthesis of glufosinate for the formation of the second phosphor-carbon bond. For example, H₃CPH(O)OR (R═Alkyl) reacts with 1-cyano allyl acetate in an addition reaction to provide an intermediate. Subsequent exchange of the acetate substitent with ammonia and hydrolysis of the cyano group and the ester group of the phosphinic acid moiety give glufosinate.

Acrylic acid ester is a cheaper alternative starting material. It can react with the ester of P-methyl phosphinic acid to 3-[alkoxy(methyl)phosphinyl]propionic acid alkyl ester. Claisen reaction of this diester with diethyl oxalate, hydrolysis and decarboxylation provide the corresponding α-keto acid, which can be reductively aminated to give glufosinate.

These and further synthetic routes towards glufosinate are also described in the art, e.g. in WO 1999/009039 A1, EP 0 508 296 A1.

A general disadvantage of all synthetic routes to glufosinate is that the obtained glufosinate is a racemic mixture. However, as there is no herbicidal activity of the D-enantiomer, L-glufosinate [hereinafter “LGA”; CAS-Nr. 35597-44-5; other names “(S)-glufosinate”, “(−)-glufosinate”] is the enantiomer of economical interest.

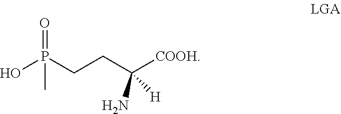

For example, CN 111662325 A discloses a synthetic pathway in which L-homoserine is reacted to the respective hydantoin, followed by addition of methane phosphor dichloride, which results in a methane phosphane which is disubstituted with L-homoserine hydantoin. After a final Arbuzov reaction and hydrolysis, LGA is obtained. Although the disclosure describes a high enantiomeric excess (=“ee”), this ee could not be reproduced by the inventors of the present invention. A reason could be that during this synthesis pathway, strong acidic (HCl for saponification of the hydantoin phosphor bond) and alkaline conditions (NaOH, 100° C. for ring opening) are applied. Such conditions usually lead to racemization, as described by M. Bovarnick & H. T. Clarke, Journal of the American Chemical Society 1938, 60, 2426-2430, by R. A. Lazarus, J. Org. Chem. 1990, 55, 4755-4757, and by A. S. Bommarius, M. Kottenhahn, H. Klenk, K. Drauz: “A direct route from hydantoins to D-amino acids employing a resting cell biocatalyst with D-hydantoinase and D-carbamoylase activity” on page 164 and 167 in “Microbial Reagents in Organic Synthesis” Series C: Mathematical and Physical Sciences-Vol. 381, S. Servi (Ed.), 1992, Springer Science+Business Media, B. V., Dordrecht.

For enantioselective syntheses of LGA, chemical and enzymatic pathways are described in the art.

WO 2020/145513 A1 and WO 2020/145514 A1 describe a chemical route to LGA. In this route, an L-homoserine derivative such as O-acetyl L-homoserine or O-succinyl L-homoserine is used as starting material and L-glufosinate is obtained by a sequence of reactions including lactonization and halogenation.

WO 2020/145627 A1 describes a similar route, wherein during halogenation, a bromo derivative is obtained.

The route disclosed by CN 106083922 A resembles these synthetic pathways but starts off from L-methionine.

CN 108516991 A describes another synthetic pathway to LGA, starting from the azeotropic dehydration of L-homoserine to give L-3,6-bis(2-haloethyl)-2,5-diketopiperazine, followed by introduction of a methylphosphonate diester group, and hydrolysis.

WO 2017/151573 A1 discloses a two-step enzymatic synthesis of LGA from D-glufosinate. In the first step, D-glufosinate is oxidatively deaminated to give 2-oxo-4-[hydroxy(methyl)phosphinoyl]-butyric acid (“PPO”), followed by the specific amination of PPO to LGA as the second step. The first step is carried out by catalysis of a D-amino acid oxidase, the second step is catalyzed by a transaminase.

WO 2020/051188 A1 discloses a similar method of converting racemic glufosinate to the L-enantiomer. In addition, it discloses a step in which the α-ketoacid or ketone byproduct formed during amination of PPO with an amine donor is converted by ketoglutarate decarboxylase to further shift the equilibrium to LGA.

WO 2019/018406 A1 discloses a method of purifying LGA from a mixture comprising LGA and glutamate. Glutamate is converted to pyroglutamate enzymatically by glutaminyl-peptidyl cyclotransferase, and LGA is then purified from the resulting mixture with an ion-exchange resin.

WO 2013/072486 A1 disclose hydantoinase mutants which have a greater activity towards D-amino acids.

WO 00/58449 A1 disclose hydantoinase mutants which have a greater activity towards L-amino acids.

The object of the present invention is to provide a further enzymatic process for producing glufosinate or glufosinate P-alkyl esters. This process should provide products with a high excess of the L-enantiomer over the D-enantiomer. Moreover, this process should allow to use new substrates which heretofore were not used in the enzymatic synthesis of glufosinate or glufosinate esters. Moreover, there is a need in the art for an enantioselective method for the production of L-glufosinate or L-glufosinate esters from starting materials comprising D- and L-enantiomers, such as racemic mixtures, in a minimal amount of synthetic steps.

### 2. Short Summary of the Invention

Namely, it was surprisingly found that the desired L-glufosinate P-alkyl esters can be produced by an enzymatically catalyzed reaction from the corresponding carbamoylates of formula L-(II), wherein R is an alkyl group or an aryl group:

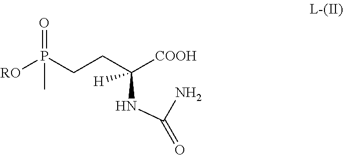

Such carbamoylates were not used in the enzymatically catalyzed production of L-glufosinate or its P-alkyl esters before. This finding was even more surprising, as it turned out that the presence of an alkyl group or aryl group for R in formula L-(II) is mandatory, as there is no corresponding reaction of compounds according to formula L-(II) in which R═H.
α.1) The present invention hence solves the problems mentioned above by providing in a first aspect a method for the production of a glufosinate P-alkyl ester according to formula L-(I), comprising a step (c) in which a compound according to the above-mentioned formula L-(II) is reacted to give a compound according to formula L-(I):

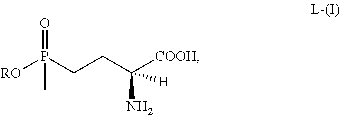

wherein the reaction according step (c) is catalyzed by a carbamoylase E₁, preferably by an L-enantioselective carbamoylase E₁ (“L-enantioselective carbamoylase”=“L-carbamoylase”) and wherein R is an alkyl group or aryl group, in particular an alkyl group or phenyl or benzyl, more preferably an alkyl group, more preferably an alkyl group with 1 to 10, even more preferably with 1 to 6, even more preferably with 1 to 4 carbon atoms, even more preferably R═ethyl or n-butyl, most preferably R═n-butyl.

The L-carbamoylase E₁ is preferably categorized in the EC class 3.5.1.87.

The polypeptide sequence of the L-carbamoylase E₁ is preferably selected from SEQ ID NO: 1 and variants thereof, SEQ ID NO: 2 and variants thereof, SEQ ID NO: 3 and variants thereof, SEQ ID NO: 4 and variants thereof, SEQ ID NO: 5 and variants thereof, SEQ ID NO: 6 and variants thereof, SEQ ID NO: 7 and variants thereof, SEQ ID NO: 8 and variants thereof, SEQ ID NO: 9 and variants thereof.

α.2) In a preferred embodiment of the method according to the first aspect of the invention, the compound according to formula L-(II) that is employed in step (c) is obtained by a step (b) in which a compound according to formula L-(III) is reacted to give the compound according to formula L-(II):

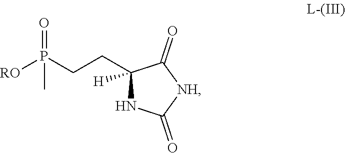

wherein the reaction according to step (b) is catalyzed by a hydantoinase E₂, preferably an L-enantioselective hydantoinase E₂ (“L-enantioselective hydantoinase”=“L-hydantoinase”), and wherein R in formula L-(III) has the same meaning as described for L-(I) above.

The hydantoinase E₂, in particular the L-hydantoinase E₂, is preferably categorized in the EC class 3.5.2.2.

The polypeptide sequence of the hydantoinase E₂, in particular the L-hydantoinase E₂, is preferably selected from SEQ ID NO: 10 and variants thereof, SEQ ID NO: 11 and variants thereof, SEQ ID NO: 12 and variants thereof, SEQ ID NO: 13 and variants thereof, SEQ ID NO: 14 and variants thereof, SEQ ID NO: 15 and variants thereof, SEQ ID NO: 16 and variants thereof.

α.3) In an even more preferred embodiment of the method according to the first aspect of the invention, the compound according to formula L-(III) that is employed in step (b) is obtained by a step (a) in which a compound according to formula D-(III) is reacted to give the compound according to formula L-(III):

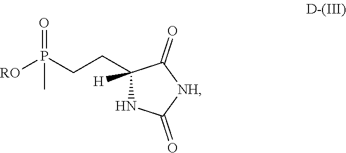

wherein the reaction according to step (a) is preferably catalyzed by a hydantoin racemase E₃, and wherein R in formula D-(III) has the same meaning as described for L-(III) above [which is the same meaning of R as described for L-(I) above].

The hydantoin racemase E₃ is preferably categorized in the EC class 5.1.99.5.

The polypeptide sequence of the hydantoin racemase E₃ in step (a) is preferably selected from SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, SEQ ID NO: 26 and variants thereof.

It was further surprisingly found that the above-mentioned finding can be advantageously used for a method for enantioselective production of an L-glufosinate P-alkyl ester from mixtures of enantiomers such as racemic mixtures.

β.1) Hence, the present invention relates in a second aspect to a method for the production of an L-glufosinate P-alkyl ester according to formula L-(I):

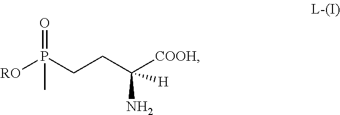

wherein R is an alkyl group or aryl group, in particular an alkyl group or phenyl or benzyl, more preferably an alkyl group, more preferably an alkyl group with 1 to 10, even more preferably with 1 to 6, even more preferably with 1 to 4 carbon atoms, even more preferably R═ethyl or n-butyl, most preferably R═n-butyl.

In this method, a mixture M_{IIIA} comprising both enantiomers L-(III) and D-(III):

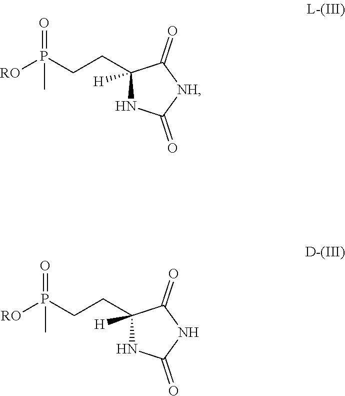

wherein R has the same meaning as described for L-(I) above, is provided [step (i)-A].

β.2) In a step (ii), the mixture M_{IIIA} is subjected to a reaction with a hydantoinase E₂, which is more preferably as defined under item α.2), giving a composition M_{II} comprising L-(II) and optionally its enantiomer D-(II), wherein L-(II) and D-(II) have the following formulae and wherein R in formulae L-(II) and D-(II) has the same meaning as described for L-(I) above:

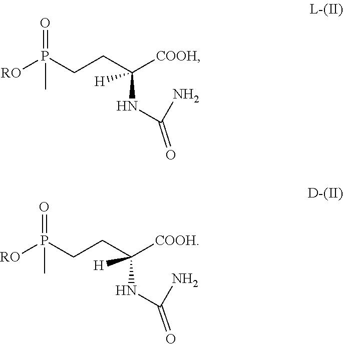

β.3) Then, in a further step (iii), M_{II} is subjected to a reaction, which is catalyzed by an L-carbamoylase E₁, which is preferably as defined under item α.1), giving a composition M_{I} comprising L-(I) and optionally its enantiomer D-(I), wherein D-(I) has the following formula and wherein R in formula D-(I) has the same meaning as described for L-(I) above:

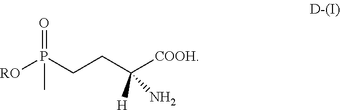

The method according to the second aspect of the invention is enantioselective, because M_{I} comprises either only L-(I) or, in case M_{I} comprises both enantiomers L-(I) and D-(I), the molar ratio of L-(I) to D-(I) in M_{I} is greater than the molar ratio of L-(III) to D-(III) in M_{IIIA}.

β.4) Furthermore, it was surprisingly found that the method for enantioselective production according to the second aspect of the invention can be further improved. Namely, in an optional step (i-B) which is carried out after step (i-A) [and which may be carried out before or concomitantly with steps (ii) and (iii), preferably concomitantly with steps (ii) and (iii)], at least a part of the compounds D-(III) comprised by the mixture M_{IIIA} are reacted, preferably enzymatically catalyzed by a hydantoin racemase E₃, into L-(III), which is the enantiomer of D-(III), giving a composition M_{IIIB} comprising L-(III) and optionally its enantiomer D-(III).

In case the reaction according to step (i-B) is enzymatically catalyzed, it is preferably catalyzed by a hydantoin racemase E₃.

The hydantoin racemase E₃ is preferably as defined under item α.3).

### 3. BRIEF DESCRIPTION OF THE DRAWINGS

FIG. 1 shows the pOM21c plasmid map.

FIG. 2 shows the pOM22c plasmid map.

FIG. 3 shows the pOM17c {Prha} plasmid map.

FIG. 4 shows the pOM17c {Prha}[amaB_Gst] plasmid map.

FIG. 5 shows the pOM17c {Prha}[atc_Ps] plasmid map.

FIG. 6 shows the pOM17c {Prha}[hyuC_Pau] plasmid map.

FIG. 7 shows the pOM17c {Prha}[hyuC_Asp (co_Ec)] plasmid map.

### 4. DETAILED DESCRIPTION OF THE INVENTION

### 4.1 Definitions

Any of the enzymes used according to any aspect of the present invention, may be an isolated enzyme. In particular, the enzymes used according to any aspect of the present invention may be used in an active state and in the presence of all cofactors, substrates, auxiliary and/or activating polypeptides or factors essential for its activity. Such factors may be metal ions such as Mn²⁺ or Co²⁺.

In particular, an enzyme according to the present application may be a carbamoylase E₁, a hydantoinase E₂, or a hydantoin racemase E₃.

“Enzymatically catalyzed” means that the respective reaction is catalyzed by an enzyme, which may be a carbamoylase E₁, a hydantoinase E₂, or a hydantoin racemase E₃.

The enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

A “polypeptide” (one or more peptides) is a chain of chemical building blocks called amino acids that are linked together by chemical bonds called peptide bonds. A protein or polypeptide, including an enzyme, may be “native” or “wild-type”, meaning that it occurs in nature or has the amino acid sequence of a native protein, respectively. These terms are sometimes used interchangeably. A polypeptide may or may not be glycosylated.

The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell.

### 4.2 Methods to Obtain Enzymes

The enzymes that can be used in the method according to the present invention can be synthesized by methods that are known to the skilled person.

One approach, which is a preferred approach according to the invention, is to express the enzyme(s) in microorganism(s) such as Escherichia coli (=“E. coli”), Saccharomyces cerevisiae, Pichia pastoris, and others, and to add the whole cells to the reactions as whole cell biocatalysts. Another approach is to express the enzyme(s), lyse the microorganisms, and add the cell lysate. Yet another approach is to purify, or partially purify, the enzyme(s) from a lysate and add pure or partially pure enzyme(s) to the reaction. If multiple enzymes are required for a reaction, the enzymes can be expressed in one or several microorganisms, including expressing all enzymes within a single microorganism.

For example, the skilled person can obtain the enzymes according to the invention by expression, in particular, overexpression, [hereinafter, “expression, in particular overexpression” is abbreviated as (over)expression“, and “express, in particular overexpress” is abbreviated as (over)express“] of these enzymes in a cell and subsequent isolation thereof, e.g. as described in DE 100 31 999 A1. Episomal plasmids, for example, are employed for increasing the expression of the respective genes. In such plasmids, the nucleic acid molecule to be (over)expressed or encoding the polypeptide or enzyme to be (over)expressed may be placed under the control of a strong inducible promoter such as the lac promoter, located upstream of the gene. A promoter is a DNA sequence consisting of about 40 to 50 base pairs which constitutes the binding site for an RNA polymerase holoenzyme and the transcriptional start point (M. Pátek, J. Holátko, T. Busche, J. Kalinowski, J. Nešvera, Microbial Biotechnology 2013, 6, 103-117), whereby the strength of expression of the controlled polynucleotide or gene can be influenced. A “functional linkage” is obtained by the sequential arrangement of a promoter with a gene, which leads to a transcription of the gene.

Suitable strong promoters or methods of producing such promoters for increasing expression are known from the literature (e.g. S. Lisser & H. Margalit, Nucleic Acid Research 1993, 21, 1507-1516; M. Pátek and J. Nesvera in H. Yukawa and M Inui (eds.), Corynebacterium glutamicum, Microbiology Monographs 23, Springer Verlag Berlin Heidelberg 2013, 51-88; B. J. Eikmanns, E. Kleinertz, W. Liebl, H. Sahm, Gene 1991, 102, 93-98). For instance, native promoters may be optimized by altering the promoter sequence in the direction of known consensus sequences with respect to increasing the expression of the genes functionally linked to these promoters (M. Pátek, B. J. Eikmanns, J. Pátek, H. Sahm, Microbiology 1996, 142, 1297-1309; M. Pátek, J. Holátko, T. Busche, J. Kalinowski, J. Nešvera, Microbial Biotechnology 2013, 6, 103-117).

Constitutive promoters are also suitable for the (over)expression, in which the gene encoding the enzyme activity is expressed continuously under the control of the promoter such as, for example, the glucose dependent deo promoter. Chemically induced promoters are also suitable, such as tac, lac, rha or trp. The most widespread system for the induction of promoters is the lac operon of E. coli. In this case, either lactose or isopropyl ß-D-thiogalactopyranoside (IPTG) is used as inducer. Also, systems using arabinose (e.g. the pBAD System) or rhamnose (e.g. E. coli KRX) are common as inducers. A system for physical induction is, for example, the temperature-induced cold shock promoter system based on the E. coli cspA promoter from Takara or Lambda PL and also osmotically inducible promoters, for example, osmB (e.g. WO 95/25785 A1).

Suitable plasmids or vectors are in principle all embodiments available for this purpose to the person skilled in the art. The state of the art describes standard plasmids that may be used for this purpose, for example the pET system of vectors exemplified by pET-3a or pET-26b(+) (commercially available from Novagen). Further plasmids and vectors can be taken, for example, pOM21 described in WO 2004/111227 A2, pOM22 described in WO 00/058449 A1 or pOM18 described in WO 2013/072486 A1 or from the brochures of the companies Novagen, Promega, New England Biolabs, Clontech or Gibco BRL. Further preferred plasmids and vectors can be found in: Glover, D. M. (1985) DNA cloning: a practical approach, Vol. I-III, IRL Press Ltd., Oxford; Rodriguez, R. L. and Denhardt, D. T (eds) (1988) Vectors: a survey of molecular cloning vectors and their uses, 179-204, Butterworth, Stoneham; Goeddel, D. V. (1990) Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York. Of these plasmids, pOM21 and pOM22 are preferred.

The plasmid vector, which contains the gene to be amplified, is then converted to the desired strain, e.g. by conjugation or transformation. The method of conjugation is described, for example, by A. Schäfer, J. Kalinowski, A. Puhler, Applied and Environmental Microbiology 1994, 60, 756-759. Methods for transformation are described, for example, in G. Thierbach, A. Schwarzer, A. Pühler, Applied Microbiology and Biotechnology 1988, 29, 356-362, L. K. Dunican & E. Shivnan, Bio/Technology 1989, 7, 1067-1070 and A. Tauch, O. Kirchner, L. Wehmeier, J. Kalinowski, A. Puhler, FEMS Microbiology Letters 1994, 123, 343-347. After homologous recombination by means of a “cross-over” event, the resulting strain contains at least two copies of the gene concerned.

The desired enzyme can be isolated by disrupting cells which contain the desired activity in a manner known to the person skilled in the art, for example with the aid of a ball mill, a French press or of an ultrasonic disintegrator and subsequently separating off cells, cell debris and disruption aids, such as, for example, glass beads, by centrifugation for 10 minutes at 13,000 rpm and 4° C. Using the resulting cell-free crude extract, enzyme assays with subsequent LC-ESI-MS detection of the products can then be carried out. Alternatively, the enzyme can be enriched in the manner known to the person skilled in the art by chromatographic methods (such as nickel-nitrilotriacetic acid affinity chromatography, streptavidin affinity chromatography, gel filtration chromatography or ion-exchange chromatography) or else purified to homogeneity. Quantification of the enzyme can be performed by methods known to the person skilled in the art, for example by determination of the concentration of the respective polypeptide of the enzyme (e.g. carbamoylase, hydantoinase and racemase) in the obtained solution by SDS page and analysis of the respective bands via the software GelQuant® (BiochemLabSolutions).

Moreover, whether or not a nucleic acid or polypeptide is (over)expressed, may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions.

4.3 V ariants

In the context of the present invention, the term “variant” with respect to polypeptide sequences refers to a polypeptide sequence with a degree of identity to the reference sequence (“sequence identity”) of at least 60%, preferably at least 70%, more preferably at least 71%, more preferably at least 72%, more preferably at least 73%, more preferably at least 74%, more preferably at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.9%. In still further particular embodiments, the degree of identity is at least 98.0%, more preferably at least 98.2%, more preferably at least 98.4%, more preferably at least 98.6%, more preferably at least 98.8%, more preferably at least 99.0%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, or at least more preferably at least 99.9%.

It goes without saying that a “variant” of a certain polypeptide sequence is not identical to the polypeptide sequence.

Such variants may be prepared by introducing deletions, insertions, substitutions, or combinations thereof, in particular in amino acid sequences, as well as fusions comprising such macromolecules or variants thereof.

Modifications of amino acid residues of a given polypeptide sequence which lead to no significant modifications of the properties and function of the given polypeptide are known to those skilled in the art. Thus for example many amino acids can often be exchanged for one another without problems; examples of such suitable amino acid substitutions are: Ala by Ser; Arg by Lys; Asn by Gln or His; Asp by Glu; Cys by Ser; Gln by Asn; Glu by Asp; Gly by Pro; His by Asn or Gin; Ile by Leu or Val; Leu by Met or Val; Lys by Arg or Gln or Glu; Met by Leu or Ile; Phe by Met or Leu or Tyr; Ser by Thr; Thr by Ser; Trp by Tyr; Tyr by Trp or Phe; Val by Ile or Leu. It is also known that modifications, particularly at the N- or C-terminus of a polypeptide in the form of for example amino acid insertions or deletions, often exert no significant influence on the function of the polypeptide.

In line with this, preferable variants according to the invention of any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, respectively, have a polypeptide sequence that comprises the complete polypeptide sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, respectively, or at least the amino acids of the respective sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 that are essential for the function, for example the catalytic activity of a protein, or the fold or structure of the protein. The other amino acids may be deleted, substituted or replaced by insertions or essential amino acids are replaced in a conservative manner to the effect that the activity of the enzyme, in particular the L-carbamoylase (SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9), hydantoinase (SEQ ID NO: 10, SEQ ID NO: 11, SEQ ID NO: 12, SEQ ID NO: 13, SEQ ID NO: 14, SEQ ID NO: 15, SEQ ID NO: 16), hydantoin racemase (SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26), is preserved.

### 3.4 Sequence Identity

The person skilled in the art is aware that various computer programs are available for the calculation of similarity or identity between two nucleotide or polypeptide sequences.

Preferred methods for determining the sequence identity initially generate the greatest alignment between the sequences to be compared. Computer programs for determining the sequence identity include, but are not limited to, the GCG program package including
GAP [J. Deveroy et al., Nucleic Acid Research 1984, 12, page 387, Genetics Computer Group University of Wisconsin, Medicine (WI)], and
BLASTP, BLASTN and FASTA (S. Altschul et al., Journal of Molecular Biology 1990, 215, 403-410). The BLAST program can be obtained from the National Center for Biotechnology Information (NCBI) and from other sources (BLAST Handbook, S. Altschul et al., NCBI NLM NIH Bethesda ND 22894; S. Altschul et al., above).

For instance, the percentage identity between two polypeptide sequences can be determined by the algorithm developed by S. B. Needleman & C. D. Wunsch, J. Mol. Biol. 1970, 48, 443-453, which has been integrated into the GAP program in the GCG software package, using either a BLOSUM62 matrix or a PAM250 matrix, a gap weight of 16, 14, 12, 10, 8, 6 or 4 and a length weight of 1, 2, 3, 4, 5 or 6. The person skilled in the art will recognize that the use of different parameters will lead to slightly different results, but that the percentage identity between two polypeptide overall will not be significantly different. The BLOSUM62 matrix is typically used applying the default settings (gap weight: 12, length weight: 1).

In the context of the present invention, a sequence identity of 60% according to the above algorithm means 60% homology. The same applies to higher sequence identities.

Most preferably, the degree of identity between sequences is determined in the context of the present invention by the programme “Needle” using the substitution matrix BLOSUM62, the gap opening penalty of 10, and the gap extension penalty of 0.5. The Needle program implements the global alignment algorithm described in S. B. Needleman & C. D. Wunsch, J. Mol. Biol. 1970, 48, 443-453. The substitution matrix used according to the present invention is BLOSUM62, gap opening penalty is 10, and gap extension penalty is 0.5. The preferred version used in the context of this invention is the one presented by F. Madeira, Y. M. Park, J. Lee, N. Buso, T. Gur, N. Madhusoodanan, P. Basutkar, A. R. N. Tivey, S. C. Potter, R. D. Finn, Nucleic Acids Research 2019, 47, W636-W641, Web Server issue (preferred version accessible online on Jun. 16, 2021 via https://www.ebi.ac.uk/Tools/psa/emboss_needle/).

In a particular embodiment, the percentage of identity of an amino acid sequence of a polypeptide with, or to, a reference polypeptide sequence is determined by
i) aligning the two amino acid sequences using the Needle program, with the BLOSUM62 substitution matrix, a gap opening penalty of 10, and a gap extension penalty of 0.5;
ii) counting the number of exact matches in the alignment;
iii) dividing the number of exact matches by the length of the longest of the two amino acid sequences, and
iv) converting the result of the division of iii) into percentage.

### 4.5 First Aspect: Method for Production of a Glufosinate Alkyl Ester

### 4.5.1 Step (c)

### 4.5.1.1 Enzymatic Catalysis of Step (c)

The present invention relates in a first aspect to a method for the production of an L-glufosinate P-alkyl ester according to formula L-(I):

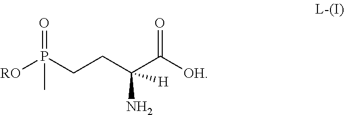

The method according to the first aspect of the invention comprises a step (c).

In step (c), a compound according to formula L-(II) is reacted to give a compound according to formula L-(I). Formula L-(II) has the following structure:

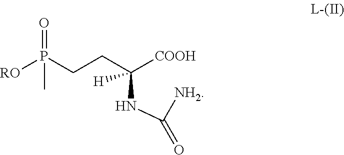

The reaction according step (c) is enzymatically catalyzed, namely it is catalyzed by a carbamoylase E₁.

In formulae L-(II) and L-(I), R is an alkyl group or an aryl group. In particular, R is selected from the group consisting of alkyl group, phenyl group, benzyl group. Preferably, R is an alkyl group, more preferably an alkyl group with 1 to 10, even more preferably with 1 to 6, even more preferably with 1 to 4 carbon atoms. Even more preferably R═ethyl or n-butyl, most preferably R═n-butyl.

### 4.5.1.2 Enantioselective and Enantiospecific Catalysis of Step (c)

Step (c) of the method according to the first aspect of the present invention is preferably L-enantioselective, even more preferably L-enantiospecific.

In such a preferred embodiment, L-(II) is in particular employed in step (c) as a mixture M_{II} comprising, besides L-(II), the enantiomer of L-(II), which is D-(II), wherein D-(II) has the following structure, wherein R in D-(II) has the same meaning as defined for L-(II) and wherein R in L-(II) and D-(II) is the same:

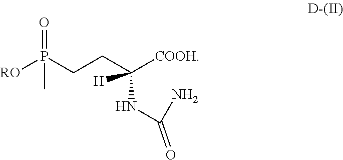

For such mixtures M_{II}, a parallel reaction according to step (c)* may be observed. Namely, in the reaction according to step (c)*, D-(II), i.e. the enantiomer of L-(II), in mixture M_{II} is reacted to give a compound according to formula D-(I):

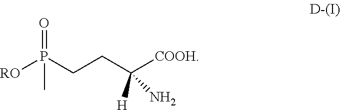

In case that step (c) is “L-enantioselective”, this means that in case a mixture M_{II} is employed in step (c), then there is either no reaction according to step (c)* or, in case there is a reaction according to step (c)*, then the rate of reaction according to step (c)* is lesser than the rate of the reaction according to step (c).

Step (c) is “L-enantiospecific”, if the rate of reaction according to step (c)* is essentially zero, i.e. there is no reaction according to step (c)*.

In a preferred embodiment, the mixture M_{II} is a racemic mixture of enantiomer L-(II) and enantiomer D-(II), meaning that the molar ratio of enantiomer L-(II) to enantiomer D-(II) is essentially 1:1.

In other preferred embodiments, the molar ratio of enantiomer L-(II) to enantiomer D-(II) in mixture M_{II} is in the range of from 3:2 to 1:99, more preferably in the range of from 1.01:1 to 1:99, more preferably in the range of from 1:1 to 1:99, more preferably in the range of from 1:1.01 to 1:99, more preferably in the range of from 1:1.01 to 1:9, more preferably in the range of from 1:1.01 to 1:8, more preferably in the range of from 1:1.01 to 1:3.

Alternatively, enantiomer D-(II) is comprised in an excess to L-(II) in mixture M_{II}, meaning that, while L-(II) is present in the mixture M_{II}, the molar ratio of enantiomer L-(II) to enantiomer D-(II) in mixture M_{II} is <1:1, preferably <0.9:1, more preferably <0.75:1, more preferably <0.5:1, more preferably <0.2:1, more preferably <0.1:1, more preferably <0.01:1.

Step (c) is in particular L-enantioselective, if it is catalyzed by an L-carbamoylase E₁, which may be determined by the skilled person as set forth under 4.5.4.3.

In case step (c) is L-enantioselective, the reaction according to step (c) proceeds preferably at a reaction rate that is at least 2 times greater, preferably at least 10 times greater, more preferably at least 100 times greater, even more preferably at least 103 times greater, even more preferably at least 104 times greater, even more preferably at least 105 times greater than the reaction rate at which step (c)* proceeds.

To quantify the factor at which the reaction rate of step (c) proceeds compared to the reaction rate of step (c)*, the following test may be carried out:

(1) An equimolar mixture [molar ratio of the two enantiomers L-(II) and D-(II) is 1:1] is subjected to the respective reactions conditions and the development of the two products L-(I) and D-(I) is monitored over time (e.g. by LC-MS as set forth under item 5.4).

(2) When n_{LI10}=10 mol-% of the initially employed L-(II) has reacted to the product L-(I), the amount of D-(I) that was formed by reaction from D-(II) [in mol-% relative to the initially employed D-(II)] is measured (=n_{DI10}).

(3) The ratio of n_{LI10}/n_{DI10}=10/n_{DI10} gives the factor at which the reaction rate of step (c) proceeds compared to the reaction rate of step (c)*.
→ If n_{LI10}/n_{DI10}>1, the reaction rate of step (c) is greater than the reaction rate of step (c)*.
→ If n_{LI10}/n_{DI10}<1, the reaction rate of step (c) is lesser than the reaction rate of step (c)*.
→ If n_{LI10}/n_{DI10}=1, the reaction rates of steps (c) and (c)* are the same.

### 4.5.2 Carbamoylases

The reaction according to step (c) of the method according to the first aspect of the invention is catalyzed by a carbamoylase E₁.

Namely, it was surprisingly found that carbamoylases accept compounds of formula L-(II) as substrates and convert them to products L-(I), and hence can be used to catalyze the reaction according to step (c). This finding is of high scientific and economic value, as it opens new synthetic routes based on new starting materials for the production of L-glufosinate P-alkyl esters and L-gluofsinate. Even more surprisingly, it was found that L-glufosinate carbamoylate, i.e. the compound according to L-(II), in which R═H, does not undergo reaction by carbamoylases to give L-glufosinate.

In nature, carbamoylases generally catalyze the following reaction <1>, wherein R^{X} may be an organic residue, e.g. a side chain of one of the naturally occurring amino acids.

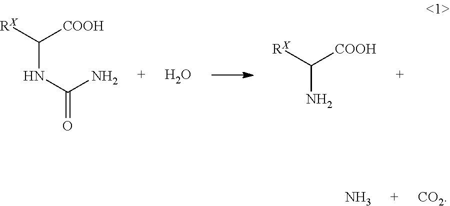

It was now surprisingly found that carbamoylases also accept substrates in which

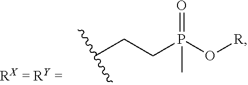

wherein R has the above meaning and wherein preferably

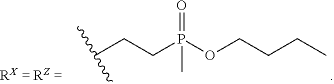 [SMILES: *CCP(C)(=O)OCCCC]

Surprisingly, they do not accept substrates in which

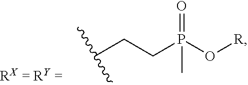

wherein R═H.

In the context of the present invention, a “carbamoylase E₁” is a carbamoylase that catalyzes the following reaction <1A> of a carbamoyl substrate S_{L} to the respective amino acid product P_{L}, wherein R^{X}=R^{Y} and preferably R^{X}=R^{Z}.

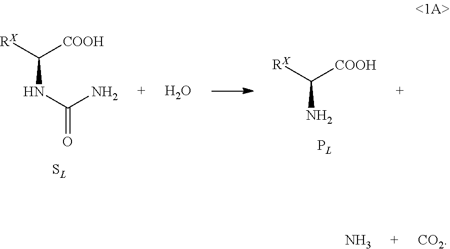

In particular, the carbamoylase E₁ is a “L-carbamolyase”, i.e. it has a greater catalytic activity for reaction <1A> than for reaction <1B>, wherein the substrate Sp in the reaction <1B> is the enantiomer of the substrate S_{L} in the reaction <1A>:

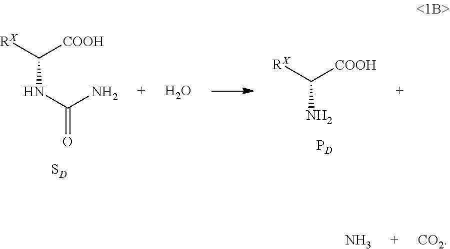

As an L-carbamoylase has a higher catalytic activity for reaction <1A> than for reaction <1B>, it is “L-enantioselective”. An L-carbamoylase that has no catalytic activity for reaction <1B> and thus only has catalytic activity for reaction <1A> is “L-enantiospecific”.

A “D-carbamoylase” is defined as a carbamoylase which is “D-enantioselective”, i.e. it has a higher catalytic activity for reaction <1B> than for reaction <1A>. A D-carbamoylase that does not catalyze reaction <1A> and thus only has catalytic activity for reaction <1B> is “D-enantiospecific”.

A carbamoylase which has the same catalytic activity for reaction <1B> as for reaction <1A>, is referred to as “non-enantioselective carbamoylase”.

For determination whether a carbamoylase may be denoted as “L-carbamoylase”, “D-carbamoylase” or “non-enantioselective carbamoylase” in the context of the present invention, the procedure according to Assay B (item 4.5.4) may preferably be used.

The carbamoylase E₁, in particular the L-carbamoylase E₁, that may be used in step (c) of the method according to the first aspect of the invention may originate from Achromobacter S p, in particular Achromobacter xylosoxidans; Agrobacterium S p., in particular Agrobacterium tumefaciens; Arthrobacter S p., in particular Arthrobacter crystallopoietes, Arthrobacter aurescens, Arthrobacter S p. BT801; Bacillus S p., in particular Bacillus fordii; Blastobacter S p.; Bradyrhizobium sp., in particular Bradyrhizobium japonicum; Brevibacillus S p., in particular Brevibacillus reuszeri; Comamonas S p.; Ensifer S p., in particular Ensifer adhaerens; Flavobacterium S p. Geobacillus S p., in particular Geobacillus kaustophilus, Geobacillus stearothermophilus; Microbacterium S p., in particular Microbacterium liquefaciens S train AJ3912; Paenarthrobacter S p., in particular Paenarthrobacter aurescens; Pasteurella S p.; Pseudomonas S p.; Ralstonia S p., in particular Ralstonia pickettii; Sinorhizobium S p., in particular Sinorhizobium meliloti.

An L-carbamoylase E₁ suitable for the method according to the present invention may be the enzyme HyuC, which originates from Arthrobacter. Other enzymes are AmaB, AtcC, Inc, SinmeB_2280.

WO 01/23582 A1 discloses an example of an enzyme having carbamoylase activity according to the invention.

The carbamoylase E₁ that may be used in step (c) of the method according to the present invention may be an L-carbamoylase categorized in the EC class EC 3.5.1.87.

L-carbamoylase enzymes are for example described by J. Ogawa, H. Miyake, S. Shimizu, Appl. Microbiol. Biotechnol. 1995 43, 1039-1043 and in WO 01/23582 A1.

A L-carbamoylase E₁ that may preferably be used in step (c) according to the first aspect of the invention may originate from Arthrobacter S p., in particular Arthrobacter crystallopoietes, Arthrobacter aurescens, Arthrobacter S p. BT801, Arthrobacter aurescens DSM 3747; Bacillus S p., in particular Bacillus fordii; Geobacillus S p., in particular Geobacillus stearothermophilus, Geobacillus kaustophilus; Microbacterium S p., in particular Microbacterium liquefaciens S train AJ3912; Paenarthrobacter S p., in particular Paenarthrobacter aurescens; Pseudomonas S p., in particular Pseudomonas S p. QR-101; Sinorhizobium S p., in particular Sinorhizobium meliloti. Even more preferably, the L-carbamoylase E₁ that may preferably be used in step (c) according to the first aspect of the invention may originate from Arthrobacter S p., in particular Arthrobacter crystallopoietes, Arthrobacter aurescens, Arthrobacter S p. BT801, Arthrobacter aurescens DSM 3747, most preferably from Arthrobacter aurescens DSM 3747.

The respective sequences can be derived from databases such as the Braunschweig Enzyme Database (BRENDA, Germany, available under www.brenda-enzymes.org/index.php), the National Center for Biotechnological Information (NCBI, available under https://www.ncbi.nlm.nih.gov/) or the Kyoto Encyclopedia of Genes and Genomes (KEGG, Japan, available under www.https://www.genome.jp/kegg/).

The following table 1 gives preferred examples for polypeptide sequences of L-carbamoylases E₁ that may be preferably used in step (c) of the method according to the first aspect of the invention. The genes encoding the respective L-carbamoylase E₁ and the respective accession code are indicated as far as known.

TABLE 1
L-Carbamoylases (EC 3.5.1.87)

| | | GenBank/ | SEQ ID NO: |
|---|---|---|---|
| | | UniProt | of the |
| Strain | Gene name | accession | polypeptide |
| Arthrobacter | hyuC | | SEQ ID NO: 1 |
| aurescens DSM 3747 | | | |
| Geobacillus | amaB | Q53389 | SEQ ID NO: 2 |
| stearothermophilus | | | |
| Pseudomonas | atcC | H9B8T5 | SEQ ID NO: 3 |
| sp. QR-101 | | | |
| Geobacillus | Inc | Q8GQG5 | SEQ ID NO: 4 |
| kaustophilus | | | |
| Paenarthrobacter | hyuC | Q9F464 | SEQ ID NO: 5 |
| aurescens | | | |
| Sinorhizobium meliloti | SinmeB_2280 | A0A0E0UEY4 | SEQ ID NO: 6 |
| Bacillus fordii | not assigned | ABL14248 | SEQ ID NO: 7 |
| strain MH602 | | | |
| Arthrobacter | hyuC | AAL55413 | SEQ ID NO: 8 |
| sp. BT801 | | | |
| Microbacterium | not assigned | | SEQ ID NO: 9 |
| liquefaciens | | | |
| strain AJ3912 | | | |

In a preferred embodiment of the method according to the first aspect of the present invention, the reaction according to step (c) is catalyzed by an L-carbamoylases E₁, wherein the polypeptide sequence of E₁ is selected from the group consisting of SEQ ID NO: 1 and variants thereof, SEQ ID NO: 2 and variants thereof, SEQ ID NO: 3 and variants thereof, SEQ ID NO: 4 and variants thereof, SEQ ID NO: 5 and variants thereof, SEQ ID NO: 6 and variants thereof, SEQ ID NO: 7 and variants thereof, SEQ ID NO: 8 and variants thereof, SEQ ID NO: 9 and variants thereof, preferably SEQ ID NO: 1 and variants thereof.

4.5.3 Assays A_{L} and A_{D} for determining carbamoylase activity

The skilled person is aware of carbamoylases, in particular L-carbamoylases, that may be used in step (c) of the method according to the first aspect of the invention.

In particular, Assay A_{L}, described in the following, may be used to determine carbamolyase and L-carbamoylase activity of a given enzyme E_{X} and may advantageously be used according to the invention to determine carbamoylase and L-carbamoylase activity in variants of SEQ ID NO: 1, variants of SEQ ID NO: 2, variants of SEQ ID NO: 3, variants of SEQ ID NO: 4, variants of SEQ ID NO: 5, variants of SEQ ID NO: 6, variants of SEQ ID NO: 7, variants of SEQ ID NO: 8, variants of SEQ ID NO: 9.

For comparative reasons, Assay A_{D} may be used to determine D-carbamoylase activity of a given enzyme E_{X}.

For the purpose of Assay A_{L} and Assay A_{D}, the molar mass of the enzyme E_{X} to be tested is calculated as the molar mass of the polypeptide sequence of E_{X}.

### 4.5.3.1 Assay A_{L}:

To 0.9 ml of an aqueous reaction solution (phosphate buffer, pH 7.2, 10 mM MnCl₂), containing 50 mM of an n-butyl P-ester of carbamoyl glufosinate according to formula L-(II), wherein R═n-butyl, are added 400 nmol of E_{X} in 0.1 ml aqueous phosphate buffer (pH 7.2, 10 mM MnCl₂). The resulting solution is incubated at 25° C., and the pH is held at pH 7.2 by addition of 0.5 M NaOH. After 300 minutes, the reaction is stopped by addition of 2 M HCl to achieve a pH of 2.5, and the molar amount of the respective LGA P-(n-butyl) ester according to formula L-(I), wherein R═n-butyl, is determined. L-(I), i.e. wherein R═n-butyl, may be detected by the LC-MS method as described in the example section (item 5.4) for detection of LGA.

### 4.5.3.2 Assay A_{D}:

To 0.9 ml of an aqueous reaction solution (phosphate buffer, pH 7.2, 10 mM MnCl₂), containing 50 mM of an n-butyl P-ester of carbamoyl glufosinate according to formula D-(II), wherein R═n-butyl, are added 400 nmol of E_{X} in 0.1 ml aqueous phosphate buffer (pH 7.2, 10 mM MnCl₂). The resulting solution is incubated at 25° C., and the pH is held at pH 7.2 by addition of 0.5 M NaOH. After 300 minutes, the reaction is stopped by addition of 2 M HCl to achieve a pH of 2.5, and the molar amount of the respective D-glufosinate P-(n-butyl) ester D-(I), wherein R═n-butyl, is determined. D-(I), wherein R═n-butyl, may be detected by the LC-MS method described in the example section (item 5.4) for detection of LGA.

### 4.5.4 Assay B for Identifiying Carbamoylases, L-Carbamoylases, D-Carbamoylases, L- and D-Enantiospecificity

The carbamoylase E₁ according to the invention is preferably an L-carbamoylase, more preferably L-enantiospecific.

Whether a given enzyme E_{X} may be considered a carbamoylase E₁, in particular an L-carbamoylase, may be determined in the context of the present invention by the following Assay B.

### 4.5.4.1 Assay B:

B-1. Firstly, Assay A_{L} as set forth under item 4.5.3.1 is conducted, and the obtained molar amount of the compound of the formula L-(I), wherein R═n-butyl, is determined according to Assay A_{L}.

B-2. Secondly, Assay A_{D} as set forth under item 4.5.3.2 is conducted, and the obtained molar amount of the compound of the formula D-(I), wherein R═n-butyl, is determined according to Assay A_{D}.

B-3. Then, step B-1 is repeated, except that instead of the addition of 400 nmol E_{X} in 0.1 ml aqueous phosphate buffer (pH 7.2, 10 mM MnCl₂), 0.1 ml aqueous phosphate buffer (pH 7.2, 10 mM MnCl₂) without E_{X} is added.

B-4. Then, step B-2 is repeated, except that instead of the addition of 400 nmol E_{X} in 0.1 ml aqueous phosphate buffer (pH 7.2, 10 mM MnCl₂), 0.1 ml aqueous phosphate buffer (pH 7.2, 10 mM MnCl₂) without E_{X} is added.

### 4.5.4.2 Carbamoylase Activity

If the molar amount of the compound of the formula L-(I), wherein R═n-butyl, that is determined in step B-1, is greater than the molar amount of the compound of the formula L-(I), wherein R═n-butyl, that is determined in step B-3, then E_{X} is deemed to have carbamoylase activity, and hence may be considered a carbamoylase E₁ in the context of the invention.

### 4.5.4.3 L-Carbamoylase Activity

### 4.5.4.3.1 L-Carbamoylases

(i) If the molar amount of the compound of formula L-(I), wherein R═n-butyl, that is determined in step B-1, is greater than the molar amount of the compound of formula L-(I), wherein R═n-butyl, that is determined in step B-3, and
(ii) if the molar amount of the compound of formula D-(I), wherein R═n-butyl, that is determined in step B-2, is greater than or the same as the molar amount of the compound of formula D-(I), wherein R═n-butyl, that is determined in step B-4, and
(iii) if, in addition, the molar amount of the compound of formula of L-(I), wherein R═n-butyl, that is determined in step B-1, is greater than the molar amount of the compound of formula of D-(I), wherein R═n-butyl, that is determined in step B-2,
→ then E_{X} is deemed to have L-carbamoylase activity, and hence may be considered an L-carbamoylase in the context of the invention. In this case, E_{X} is deemed to be “L-enantioselective” in the context of the invention.

For the sake of clarity it is pointed out that condition (iii) is automatically fulfilled in those cases in which the molar amount of the compound of formula D-(I), wherein R═n-butyl, that is determined in step B-2, is the same as the molar amount of the compound of formula D-(I), wherein R═n-butyl, that is determined in step B-4.
(i) If the molar amount of the compound of formula L-(I), wherein R═n-butyl, that is determined in step B-1, is greater than the molar amount of the compound of formula L-(I), wherein R═n-butyl, that is determined in step B-3, and
(ii) if the molar amount of the compound of formula D-(I), wherein R═n-butyl, that is determined in step B-2 is the same as the molar amount of the compound of formula D-(I), wherein R═n-butyl, that is determined in step B-4,
→ then E_{X} is deemed to have L-carbamoylase activity, and hence may be considered to be an L-carbamoylase in the context of the invention. In this case, E_{X} is not only “L-enantioselective”, but also “L-enantiospecific” in the context of the invention.

For L-carbamyolases E_{X} that are not L-enantiospecific, the L-enantioselectivity may then be quantified by dividing the molar amount of the compound of formula L-(I), wherein R═n-butyl, that is determined in step B-1, by the molar amount of the compound of formula D-(I), wherein R═n-butyl, that is determined in step B-2, and then multiplying the obtained value by 100, giving the L-enantioselectivity of E_{X} in %. 4.5.4.3.2 D-carbamoylases
(i) If the molar amount of the compound of formula L-(I), wherein R═n-butyl, that is determined in step B-1, is greater than or the same as the molar amount of the compound of formula L-(I), wherein R═n-butyl, that is determined in step B-3, and
(ii) if the molar amount of the compound of formula D-(I), wherein R═n-butyl, that is determined in step B-2, is greater than the molar amount of the compound of formula D-(I), wherein R═n-butyl, that is determined in step B-4, and
(iii) if, in addition, the molar amount of the compound of formula D-(I), wherein R═n-butyl, that is determined in step B-2 is greater than the molar amount of the compound of formula L-(I), wherein R═n-butyl, that is determined in step B-1,
→ then E_{X} is deemed to have D-carbamoylase activity, and hence may be considered a D-carbamoylase. In this case, E_{X} is “D-enantioselective” in the context of the invention.

For the sake of clarity it is pointed out that condition (iii) is automatically fulfilled in those cases in which the molar amount of the compound of formula L-(I), wherein R═n-butyl, that is determined in step B-1 is the same as the molar amount of the compound of formula L-(I), wherein R═n-butyl, that is determined in step B-3.
(i) If the molar amount of the compound of formula L-(I), wherein R═n-butyl, that is determined in step B-1, is the same as the molar amount of the compound of formula L-(I), wherein R═n-butyl, that is determined in step B-3, and
(ii) if the molar amount of the compound of formula D-(I), wherein R═n-butyl, that is determined in step B-2, is greater than the molar amount of the compound of formula D-(I), wherein R═n-butyl, that is determined in step B-4,
→ then E_{X} is deemed to have D-carbamoylase activity, and hence may be considered to be a D-carbamoylase. In this case, E_{X} is not only “D-enantioselective”, but also “D-enantiospecific” in the context of the invention.

For D-carbamoylases E_{X}, that are not D-enantiospecific, the D-enantioselectivity may then be quantified by dividing the molar amount of the compound of formula D-(I), wherein R═n-butyl, that is determined in step B-2 by the molar amount of the compound of formula L-(I), wherein R═n-butyl, that is determined in step B-1 and then multiplying the obtained value by 100, giving the D-enantioselectivity of E_{X} in %.

### 4.5.4.3.3 Non-Enantioselective Carbamoylases

(i) If the molar amount of the compound of formula L-(I), wherein R═n-butyl, that is determined in step B-1, is greater than the molar amount of the compound of formula L-(I), wherein R═n-butyl, that is determined in step B-3, and
(ii) if the molar amount of the compound of formula D-(I), wherein R═n-butyl, that is determined in step B-2 is greater than the molar amount of the compound of formula D-(I), wherein R═n-butyl, that is determined in step B-4, and
(iii) if, in addition, the molar amount of the compound of formula D-(I), wherein R═n-butyl, that is determined in step B-2 is the same as the molar amount of the compound of formula L-(I), wherein R═n-butyl, that is determined in step B-1,
→ then E_{X} is deemed to be a non-enantioselective carbamoylase in the context of the invention. In this case, E_{X} is “non-enantioselective”.

### 4.5.5 Assay C for Identifying Preferred Carbamoylase Variants of SEQ ID NOs: 1-9

### 4.5.5.1 L- and D-Enantioselective Carbamoylase Variants

An enzyme, the polpypetide sequence of which is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 has carbamoylase, and L-carbamoylase activity.

In a preferred embodiment of the method according to the first aspect of the invention, the polypeptide sequence of the L-carbamoylase E₁ is selected from the group consisting of SEQ ID NO: 1 and variants thereof, SEQ ID NO: 2 and variants thereof, SEQ ID NO: 3 and variants thereof, SEQ ID NO: 4, and variants thereof, SEQ ID NO: 5 and variants thereof, SEQ ID NO: 6 and variants thereof, SEQ ID NO: 7, and variants thereof, SEQ ID NO: 8 and variants thereof, SEQ ID NO: 9 and variants thereof.

In an even more preferred embodiment of the method according to the first aspect of the invention, the polypeptide sequence of the L-carbamoylase E₁ is selected from the group consisting of SEQ ID NO: 1 and variants thereof, SEQ ID NO: 2 and variants thereof, SEQ ID NO: 3 and variants thereof, SEQ ID NO: 5 and variants thereof, SEQ ID NO: 8 and variants thereof, more preferably SEQ ID NO: 1 and variants thereof.

The term “variant” is defined under item 4.3.

In the context of the invention, an enzyme E₁, the polypeptide sequence of which is a variant of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 has carbamoylase activity, preferably L-carbamoylase activity, more preferably is L-enantiospecific.

Whether a given enzyme E_{X}, the polypeptide sequence of which is a variant of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, has carbamoylase activity, L carbamoylase activity and/or is L-enantiospecific may be determined as set forth under items 4.5.4.2 and 4.5.4.3.1, respectively.

The carbamoylase activity of a given L-carbamoylase E_{1V}, the polypeptide sequence of which is a variant of one of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, relative to the carbamoylase activity of an L-carbamoylase E_{1S}, wherein the polypeptide sequence of E_{1S} is selected from of one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, may be quantified in the context of the present invention by the following Assay C:

### 4.5.5.2 Assay C

C-1 Assay A_{L} as set forth under item 4.5.3.1 is conducted, wherein E_{1S} is the enzyme to be tested. The obtained molar amount of the compound according to formula L-(I), wherein R═n-butyl, is determined according to Assay A_{L}.

C-2 Step C-1 is repeated, except that, instead of E_{1S}, E_{1V} is used as the enzyme to be tested.

C-3. Then, the molar amount of the compound according to formula L-(I), wherein R═n-butyl, that is determined in step C-2, is divided by the molar amount of the compound according to formula L-(I), wherein R═n-butyl, that is determined in step C-1, and the obtained ratio is multiplied by 100, giving the carbamoylase activity of L-carbamoylase E_{1V}, relative to the carbamoylase activity of the L-carbamoylase E_{1S}, in %.

### 4.5.6 Preferred Carbamoylase Variants of SEQ ID NOs: 1-9

In the context of the invention, L-carbamoylases E₁, the polypeptide sequence of which is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, are generally denoted as “E_{1S}”.

L-carbamoylases E₁, the polypeptide sequence of which is selected from variants of a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, are generally denoted as “E_{1V}”.

In a preferred embodiment of the method according to the first aspect of the present invention, the reaction in step (c) is catalyzed by an L-carbamoylase E₁, and the polypeptide sequence of the L-carbamoylase E₁ is selected from the group consisting of SEQ ID NO: 1 and variants thereof, SEQ ID NO: 2 and variants thereof, SEQ ID NO: 3 and variants thereof, SEQ ID NO: 4 and variants thereof, SEQ ID NO: 5 and variants thereof, SEQ ID NO: 6 and variants thereof, SEQ ID NO: 7 and variants thereof, SEQ ID NO: 8 and variants thereof, SEQ ID NO: 9 and variants thereof.

More preferably, the reaction in step (c) is catalyzed by an L-carbamoylase E₁, the reaction in step (c) is catalyzed by an L-carbamoylase E₁, and the polypeptide sequence of the L-carbamoylase E₁ is selected from the group consisting of SEQ ID NO: 1 and variants of SEQ ID NO: 1, SEQ ID NO: 2 and variants thereof, SEQ ID NO: 5 and variants thereof, SEQ ID NO: 8 and variants thereof.

More preferably, the reaction in step (c) is catalyzed by an L-carbamoylase E₁, and the polypeptide sequence of the L-carbamoylase E₁ is selected from the group consisting of SEQ ID NO: 1 and variants of SEQ ID NO: 1.

### 4.5.6.1 Preferred Variants of SEQ ID NO: 1

According to the invention, the polypeptide sequence of the L-carbamoylase E₁ may also be a variant of SEQ ID NO: 1.

The L-carbamoylase E₁, the polypeptide sequence of which is SEQ ID NO: 1, is denoted as “E_{101S}”. L-carbamoylases E₁, the polypeptide sequence of which is selected from variants of SEQ ID NO: 1, are generally denoted as “E_{101V}”.

A variant of the polypeptide sequence of SEQ ID NO: 1 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 1.

The polypeptide sequence of a variant of the polypeptide sequence SEQ ID NO: 1 is not identical to SEQ ID NO: 1.

According to the invention, an L-carbamoylase E_{101V} has carbamoylase activity and L-carbamoylase activity, determined as described under items 4.5.4.2 and 4.5.4.3.1.

According to the invention, an L-carbamoylase E_{101V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{101S}, wherein the carbamoylase activity of E_{101V}, relative to the carbamoylase activity of E_{101S}, is determined by Assay C described under item 4.5.5.2.

It is even more preferable according to the invention, that an L-carbamoylase E_{101V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{101S}, wherein the carbamoylase activity of E_{101V}, relative to the carbamoylase activity of E_{101S}, is determined by Assay C described under item 4.5.5.2.

### 4.5.6.2 Preferred Variants of SEQ ID NO: 2

According to the invention, the polypeptide sequence of the L-carbamoylase E₁ may also be a variant of SEQ ID NO: 2.

The L-carbamoylase E₁, the polypeptide sequence of which is SEQ ID NO: 2, is denoted as “E_{102S}”. L-carbamoylases E₁, the polypeptide sequence of which is selected from variants of SEQ ID NO: 2, are generally denoted as “E_{102V}”.

A variant of the polypeptide sequence of SEQ ID NO: 2 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 2.

The polypeptide sequence of a variant of the polypeptide sequence SEQ ID NO: 2 is not identical to SEQ ID NO: 2.

According to the invention, an L-carbamoylase E_{102V} has carbamoylase activity and L-carbamoylase activity, determined as described under items 4.5.4.2 and 4.5.4.3.1.

According to the invention, an L-carbamoylase E_{102V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{102S}, wherein the carbamoylase activity of E_{102V}, relative to the carbamoylase activity of E_{102S}, is determined by Assay C described under item 4.5.5.2.

It is even more preferable according to the invention, that an L-carbamoylase E_{102V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{102S}, wherein the carbamoylase activity of E_{102V}, relative to the carbamoylase activity of E_{102S}, is determined by Assay C described under item 4.5.5.2.

### 4.5.6.3 Preferred Variants of SEQ ID NO: 3

According to the invention, the polypeptide sequence of the L-carbamoylase E₁ may also be a variant of SEQ ID NO: 3.

The L-carbamoylase E₁, the polypeptide sequence of which is SEQ ID NO: 3, is denoted as “E_{103S}”. L-carbamoylases E₁, the polypeptide sequence of which is selected from variants of SEQ ID NO: 3, are generally denoted as “E_{103V}”.

A variant of the polypeptide sequence of SEQ ID NO: 3 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 3.

The polypeptide sequence of a variant of the polypeptide sequence SEQ ID NO: 3 is not identical to SEQ ID NO: 3.

According to the invention, an L-carbamoylase E_{103V} has carbamoylase activity and L-carbamoylase activity, determined as described under items 4.5.4.2 and 4.5.4.3.1.

According to the invention, an L-carbamoylase E_{103V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{103S}, wherein the carbamoylase activity of E_{103V}, relative to the carbamoylase activity of E_{103S}, is determined by Assay C described under item 4.5.5.2.

It is even more preferable according to the invention, that an L-carbamoylase E_{103V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{103S}, wherein the carbamoylase activity of E_{103V}, relative to the carbamoylase activity of E_{103S}, is determined by Assay C described under item 4.5.5.2.

### 4.5.6.4 Preferred Variants of SEQ ID NO: 4

According to the invention, the polypeptide sequence of the L-carbamoylase E₁ may also be a variant of SEQ ID NO: 4.

The L-carbamoylase E₁, the polypeptide sequence of which is SEQ ID NO: 4, is denoted as “E_{104S}”. L-carbamoylases E₁, the polypeptide sequence of which is selected from variants of SEQ ID NO: 4, are generally denoted as “E_{104V}”.

A variant of the polypeptide sequence of SEQ ID NO: 4 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 4.

The polypeptide sequence of a variant of the polypeptide sequence SEQ ID NO: 4 is not identical to SEQ ID NO: 4.

According to the invention, an L-carbamoylase E_{104V} has carbamoylase activity and L-carbamoylase activity, determined as described under items 4.5.4.2 and 4.5.4.3.1.

According to the invention, an L-carbamoylase E_{104V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{104S}, wherein the carbamoylase activity of E_{104V}, relative to the carbamoylase activity of E_{104S}, is determined by Assay C described under item 4.5.5.2.

It is even more preferable according to the invention, that an L-carbamoylase E_{104V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{104S}, wherein the carbamoylase activity of E_{104V}, relative to the carbamoylase activity of E_{104S}, is determined by Assay C described under item 4.5.5.2.

### 4.5.6.5 Preferred Variants of SEQ ID NO: 5

According to the invention, the polypeptide sequence of the L-carbamoylase E₁ may also be a variant of SEQ ID NO: 5.

The L-carbamoylase E₁, the polypeptide sequence of which is SEQ ID NO: 5, is denoted as “E_{105S}”. L-carbamoylases E₁, the polypeptide sequence of which is selected from variants of SEQ ID NO: 5, are generally denoted as “E_{105V}”.

A variant of the polypeptide sequence of SEQ ID NO: 5 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 5.

The polypeptide sequence of a variant of the polypeptide sequence SEQ ID NO: 5 is not identical to SEQ ID NO: 5.

According to the invention, an L-carbamoylase E_{106V} has carbamoylase activity and L-carbamoylase activity, determined as described under items 4.5.4.2 and 4.5.4.3.1.

According to the invention, an L-carbamoylase E_{105V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{105S}, wherein the carbamoylase activity of E_{105V}, relative to the carbamoylase activity of E_{105S}, is determined by Assay C described under item 4.5.5.2.

It is even more preferable according to the invention, that an L-carbamoylase E_{106V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{105S}, wherein the carbamoylase activity of E_{105V}, relative to the carbamoylase activity of E_{105S}, is determined by Assay C described under item 4.5.5.2.

### 4.5.6.6 Preferred Variants of SEQ ID NO: 6

According to the invention, the polypeptide sequence of the L-carbamoylase E₁ may also be a variant of SEQ ID NO: 6.

The L-carbamoylase E₁, the polypeptide sequence of which is SEQ ID NO: 6, is denoted as “E_{106S}”. L-carbamoylases E₁, the polypeptide sequence of which is selected from variants of SEQ ID NO: 6, are generally denoted as “E_{106V}”.

A variant of the polypeptide sequence of SEQ ID NO: 6 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 6.

The polypeptide sequence of a variant of the polypeptide sequence SEQ ID NO: 6 is not identical to SEQ ID NO: 6.

According to the invention, an L-carbamoylase E_{106V} has carbamoylase activity and L-carbamoylase activity, determined as described under items 4.5.4.2 and 4.5.4.3.1.

According to the invention, an L-carbamoylase E_{106V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{106S}, wherein the carbamoylase activity of E_{106V}, relative to the carbamoylase activity of E_{106S}, is determined by Assay C described under item 4.5.5.2.

It is even more preferable according to the invention, that an L-carbamoylase E_{106V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{106S}, wherein the carbamoylase activity of E_{106V}, relative to the carbamoylase activity of E_{106S}, is determined by Assay C described under item 4.5.5.2.

### 4.5.6.7 Preferred Variants of SEQ ID NO: 7

According to the invention, the polypeptide sequence of the L-carbamoylase E₁ may also be a variant of SEQ ID NO: 7.

The L-carbamoylase E₁, the polypeptide sequence of which is SEQ ID NO: 7, is denoted as “E_{107S}”. L-carbamoylases E₁, the polypeptide sequence of which is selected from variants of SEQ ID NO: 7, are generally denoted as “E_{107V}”.

A variant of the polypeptide sequence of SEQ ID NO: 7 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 7.

The polypeptide sequence of a variant of the polypeptide sequence SEQ ID NO: 7 is not identical to SEQ ID NO: 7.

According to the invention, an L-carbamoylase E_{107V} has carbamoylase activity and L-carbamoylase activity, determined as described under items 4.5.4.2 and 4.5.4.3.1.

According to the invention, an L-carbamoylase E_{107V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{107S}, wherein the carbamoylase activity of E_{107V}, relative to the carbamoylase activity of E_{107S}, is determined by Assay C described under item 4.5.5.2.

It is even more preferable according to the invention, that an L-carbamoylase E_{107V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{107S}, wherein the carbamoylase activity of E_{107V}, relative to the carbamoylase activity of E_{107S}, is determined by Assay C described under item 4.5.5.2.

### 4.5.6.8 Preferred Variants of SEQ ID NO: 8

According to the invention, the polypeptide sequence of the L-carbamoylase E₁ may also be a variant of SEQ ID NO: 8.

The L-carbamoylase E₁, the polypeptide sequence of which is SEQ ID NO: 8, is denoted as “E_{108S}”. L-carbamoylases E₁, the polypeptide sequence of which is selected from variants of SEQ ID NO: 8, are generally denoted as “E_{108V}”.

A variant of the polypeptide sequence of SEQ ID NO: 8 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 8.

The polypeptide sequence of a variant of the polypeptide sequence SEQ ID NO: 8 is not identical to SEQ ID NO: 8.

According to the invention, an L-carbamoylase E_{108V} has carbamoylase activity and L-carbamoylase activity, determined as described under items 4.5.4.2 and 4.5.4.3.1.

According to the invention, an L-carbamoylase E_{108V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{108S}, wherein the carbamoylase activity of E_{108V}, relative to the carbamoylase activity of E_{108S}, is determined by Assay C described under item 4.5.5.2.

It is even more preferable according to the invention, that an L-carbamoylase E_{108V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{108S}, wherein the carbamoylase activity of E₁₀₈, relative to the carbamoylase activity of E_{108S}, is determined by Assay C described under item 4.5.5.2.

### 4.5.6.9 Preferred Variants of SEQ ID NO: 9

According to the invention, the polypeptide sequence of the L-carbamoylase E₁ may also be a variant of SEQ ID NO: 9.

The L-carbamoylase E₁, the polypeptide sequence of which is SEQ ID NO: 9, is denoted as “E_{109S}”. L-carbamoylases E₁, the polypeptide sequence of which is selected from variants of SEQ ID NO: 9, are generally denoted as “E_{109V}”.

A variant of the polypeptide sequence of SEQ ID NO: 9 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 9.

The polypeptide sequence of a variant of the polypeptide sequence SEQ ID NO: 9 is not identical to SEQ ID NO: 9.

According to the invention, an L-carbamoylase E_{109V} has carbamoylase activity and L-carbamoylase activity, determined as described under items 4.5.4.2 and 4.5.4.3.1.

According to the invention, an L-carbamoylase E_{109V} preferably has carbamoylase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the carbamoylase activity of the L-carbamoylase E_{109S}, wherein the carbamoylase activity of E_{109V}, relative to the carbamoylase activity of E_{109S}, is determined by Assay C described under item 4.5.5.2.

It is even more preferable according to the invention, that an L-carbamoylase E_{109V} has carbamoylase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the carbamoylase activity of the L-carbamoylase E_{109S}, wherein the carbamoylase activity of E_{109V}, relative to the carbamoylase activity of E_{109S}, is determined by Assay C described under item 4.5.5.2.

### 4.5.7 Preferred Method Conditions in Step (c)

The reaction in step (c) of the method according to the first aspect of the present invention may be carried out under conditions known to the skilled person.

The reaction medium is preferably aqueous, more preferably an aqueous buffer.

Exemplary buffers commonly used in biotransformation reactions and advantageously used herein include Tris, phosphate, or any of Good's buffers, such as 2-(N-morpholino)ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”). In some embodiments, ammonium can act as a buffer. One or more organic solvents can also be added to the reaction.

The buffer preferably contains metal salts, more preferably metal salts such as halogenides of metals, preferably halogenides of monovalent or bivalent or trivalent metals, preferably chlorides of monovalent or bivalent metals, preferably CoCl₂ or MnCl₂, preferably CoCl₂.

The concentration of these metal salts in the reaction medium is preferably in the range from 1 μM to 1 M, more preferably 1 mM to 100 mM, even more preferably 1 to 10 mM.

Preferably, step (c) of the method according to the first aspect of the invention is carried out in a phosphate buffer.

The pH of the reaction medium in step (c) of the method according to the first aspect of the invention is preferably in the range of from 2 to 10, more preferably in the range of from 5 to 8, more preferably 7.2 to 7.5, most preferably 7.5.

Preferably, step (c) of the method according to the first aspect of the invention is carried out at a temperature in the range of from 20° C. to 70° C., more preferably in the range of from 30° C. to 55° C., most preferably 50° C.

Preferably, the total concentration of all carbamoylases E₁ in the reaction solution in step (c) is in the range of from 1 μM to 10 mM, preferably 10 μM to 1 mM, more preferably 0.1 mM to 0.5 mM, most preferably 0.4 mM.

In alternative preferred embodiments, the total concentration of all carbamoylases E₁ in the reaction solution in step (c) is in the range of from 1 μg/l to 10 g/l, preferably 0.1 mg/l to 5 g/l, more preferably 1 mg/l to 1 g/l, more preferably 5 mg/l to 500 mg/l.

Preferably, the initial concentration of all the compounds according to formula L-(II) in the reaction medium in step (c) is in the range of from 1 μM to 1 M, preferably of from 10 μM to 0.5 M, more preferably of from 0.1 mM to 0.1 M, more preferably of from 1 mM to 10 mM, most preferably 1.25 mM.

If compounds according to formula D-(II) are present in the reaction medium in step (c), the initial concentration of all the compounds according to formula D-(II) in the reaction medium is preferably from 1% to 100% the concentration of all the compounds according to formula L-(II), more preferably 10% to 100% the concentration, even more preferably 50 to 100%, even more preferably 100% the concentration of all the compounds according to formula L-(II).

“Initial concentration of all the compounds according to formula L-(II)/D-(II)” refers to the concentration of the respective compound L-(II) or D-(II) respectively, in the reaction medium when the respective compounds are employed in step (c).

### 4.5.8 Step (b)

### 4.5.8.1 Enzymatic Catalysis of Step (b)

In a preferred embodiment of the method according to the first aspect of the invention, the compound according to formula L-(II) is obtained by a step (b) in which a compound according to formula L-(III) is reacted to give a compound according to formula L-(II):

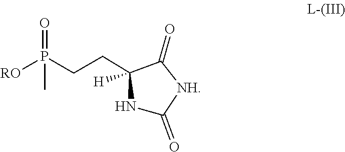

Step (b) gives the starting material for step (c), and R in L-(III) has the same meaning as described for L-(I).

The reaction according step (b) is enzymatically catalyzed, namely it is catalyzed by a hydantoinase E₂.

### 4.5.8.2 Enantioselective and Enantiospecific Catalysis of Step (b)

Step (b) of the method according to the first aspect of the present invention is preferably L-enantioselective, even more preferably L-enantiospecific.

In such a preferred embodiment, L-(III) is in particular employed in step (b) as a mixture Mill comprising, besides L-(III), the enantiomer of L-(III), which is D-(III), wherein D-(III) has the following structure, wherein R in L-(III) and D-(III) is the same:

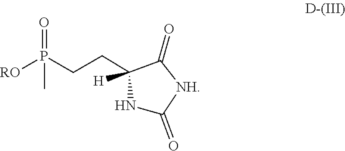

For such mixtures M_{III}, a parallel reaction according to step (b)* may be observed. Namely, in the reaction according to step (b)*, D-(III), i.e. the enantiomer of L-(III), in mixture M_{III} is reacted to give a compound according to formula D-(II):

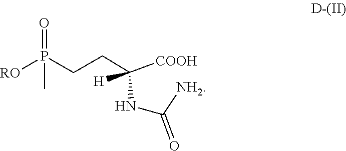

In case that step (b) is “L-enantioselective”, this means that in case a mixture Mil is employed in step (b), then there is either no reaction according to step (b)* or, in case there is a reaction according to step (b)*, then the rate of reaction according to step (b)* is lesser than the rate of the reaction according to step (b).

Step (b) is “L-enantiospecific”, if the rate of reaction according to step (b)* is essentially zero, i.e. there is no reaction according to step (b)*.

In a preferred embodiment, the mixture Mil is a racemic mixture of enantiomer L-(III) and enantiomer D-(III), meaning that the molar ratio of enantiomer L-(III) to enantiomer D-(III) is essentially 1:1.

In other preferred embodiments, the molar ratio of enantiomer L-(III) to enantiomer D-(III) in mixture M_{III} is in the range of from 3:2 to 1:99, more preferably in the range of from 1.01:1 to 1:99, more preferably in the range of from 1:1 to 1:99, more preferably in the range of from 1:1.01 to 1:99, more preferably in the range of from 1:1.01 to 1:9, more preferably in the range of from 1:1.01 to 1:8, more preferably in the range of from 1:1.01 to 1:3.

Alternatively, enantiomer D-(III) is comprised in an excess to L-(III) in mixture M_{III}, meaning that, while L-(III) is present in the mixture M_{III}, the molar ratio of enantiomer L-(III) to enantiomer D-(III) in mixture M_{III} is <1:1, preferably <0.9:1, more preferably <0.75:1, more preferably <0.5:1, more preferably <0.2:1, more preferably <0.1:1, more preferably <0.01:1.

Step (b) is in particular L-enantioselective, if it is preferably catalyzed by an L-hydantoinase E₂, which may be determined by the skilled person as set forth under 4.5.10.3.

In case step (b) is L-enantioselective, the reaction according to step (b) proceeds preferably at a reaction rate that is at least 2 times greater, preferably at least 10 times greater, more preferably at least 100 times greater, even more preferably at least 103 times greater, even more preferably at least 104 times greater, even more preferably at least 105 times greater than the reaction rate at which step (b)* proceeds.

To quantify the factor at which the reaction rate of step (b) proceeds compared to the reaction rate of step (b)*, the following test may be carried out:

(1) An equimolar mixture [molar ratio of the two enantiomers L-(III) and D-(III) is 1:1] is subjected to the respective reactions conditions and the development of the two products L-(II) and D-(II) is monitored over time (e.g. by LC-MS as set forth under item 5.4).

(2) When n_{LII10}=10 mol-% of the initially employed L-(III) has reacted to the product L-(II), the molar amount of D-(II) that was formed by reaction from D-(III) [in mol-% relative to the initially employed D-(II)] is measured (=n_{DII10}).

(3) The ratio of n_{LII10}/n_{DII10}=10/n_{DII10}gives the factor by which the reaction rate of step (b) is higher than the reaction rate of step (b)*.
→ If n_{LII10}/n_{DII10}>1, the reaction rate of step (b) is greater than the reaction rate of step (b)*.
→ If n_{LII10}/n_{DII10}<1, the reaction rate of step (b) is lesser than the reaction rate of step (b)*.
→ If n_{LII10}/n_{DII10}=1, the reaction rates of steps (b and (b)* are the same.

The reaction according to step (b) of the preferred embodiment of the first aspect of the invention is catalyzed by a hydantoinase (“dihydropyrimidinase”) E₂.

Namely, it was surprisingly found that hydantoinases accept compounds of formula L-(III) as substrates and convert them to products according to formulae L-(II), and hence catalyze the reaction according to step (b). This finding is of high scientific and economic value, as it further broadens the scope of synthetic routes based on new starting materials for the production of L-glufosinate P-alkyl esters and L-gluofsinate. Even more surprisingly, it is suggested that L-glufosinate hydantoin, i.e. the compound according to formula L-(III), in which R═H, does not undergo reaction by hydantoinases to give the respective LGA carbamoylate.

In nature, hydantoinases (“dihydropyrimidinases”) generally catalyze the reaction of 5,6-dihydrouracil to produce ureidopropionate (see the following reaction <2A>):

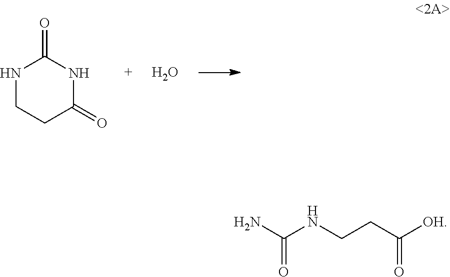 [SMILES: O=C1CCNC(=O)N1 ; O ; NC(=O)NCCC(=O)O]

They also catalyze the ring opening of monosubstituted hydantoins to give the respective carbamoyl amino acid according to the following reaction <2B>, wherein R* may be an organic residue, e.g. a side chain of one of the naturally occurring amino acids.

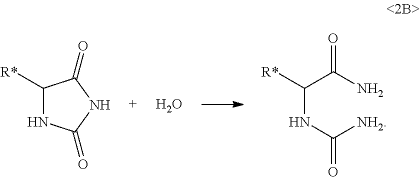

Chapter 1.3 (pages 7 to 11) of the dissertation “Untersuchungen zur Substratspezifität und Enantioselektivität mikrobieller Hydantoinasen/Investigations of substrate specificity and enantioselectivity of microbial hydantoinases” by T. Waniek, University of Stuttgart, 2000 (available under: https://elib.uni-stuttgart.de/bitstream/11682/1511/1/Diss.pdf) gives an overview over hydantoinases.

It was now surprisingly found that hydantoinases also accept substrates in which

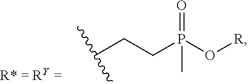

wherein R has the above meaning and wherein preferably

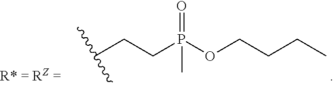 [SMILES: *CCP(C)(=O)OCCCC]

Surprisingly, they supposedly do not accept substrates in which

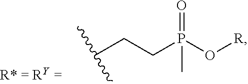

wherein R═H.

In the context of the present invention, a “hydantoinase E₂” is a hydantoinase that catalyzes the following reaction <2C> of a carbamoyl substrate S′_{L} to the respective amino acid product P′_{L}, wherein R*═R^{Y} and preferably R*═R^{Z}:

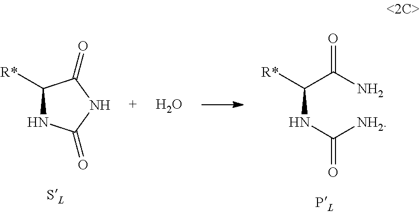

In particular, the hydantoinase E₂ is a “L-hydantoinase”, i.e. it has a greater catalytic activity for reaction <2C> than for reaction <2D>, wherein the substrate S′p in the reaction <2D> is the enantiomer of the substrate S_{L} in the reaction <2C>:

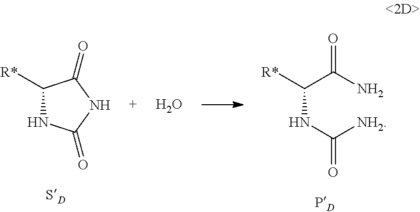

As an L-hydantoinase has a higher catalytic activity for reaction <2C> than for reaction <2D>, it is “L-enantioselective”. An L-hydantoinase that has no catalytic activity for reaction <2D> and thus only has catalytic activity for reaction <2C> is “L-enantiospecific”.

A “D-hydantoinase” is defined as a hydantoinase which is “D-enantioselective”, i.e. it has a higher catalytic activity for reaction <2D> than for reaction <2C>. A D-carbamoylase that does not catalyze reaction <2C> and thus only has catalytic activity for reaction <2D> is “D-enantiospecific”.

A hydantoinase which has the same catalytic activity for reaction <2C> as for reaction <2D>, is referred to as “non enantioselective hydantoinase”.

For determination whether a hydantoinase may be denoted as “L-hydantoinase”, “D-hydantoinase” or “non-enantioselective hydantoinase” in the context of the present invention, the procedure according to Assay E (item 4.5.10) may preferably be used.

The hydantoinase E₂, in particular the L-hydantoinase E₂, that may be used in step (b) of the preferred embodiment of the method according to the first aspect of the invention may originate from Arthrobacter S p., in particular Arthrobacter crystallopoietes, Arthrobacter aurescens, Arthrobacter S p. BT801; Alcaligenes S p., in particular Alcaligenes faecalis S ubsp. faecalis; Bacillus sp., in particular Bacillus fordii; Microbacterium S p., in particular Microbacterium liquefaciens S train AJ3912; Pseudomonas S p., in particular Pseudomonas fluorescens, Pseudomonas aeruginosa.

A hydantoinase E₂, in particular an L-hydantoinase E₂ suitable for the method according to the present invention may be the enzyme HyuH, which originates from Arthrobacter. Another enzyme may be Dht.

Even more preferably, the hydantoinase E₂, in particular the L-hydantoinase E₂, that may preferably be used in step (b) according to the first aspect of the invention may originate from Arthrobacter S p., in particular Arthrobacter crystallopoietes, Arthrobacter aurescens, Arthrobacter sp. BT801, Arthrobacter aurescens DSM 9771, most preferably from Arthrobacter aurescens DSM 9771.

A hydantoinase suitable for the method according to the present invention is described e.g. in WO 01/23582 A1 and by J. M. Clemente-Jiménez, S. Martínez-Rodríguez, F. Rodríguez-Vico, F. J. L. Heras-Vázquez, Recent Pat. Biotechnology 2008, 2, 35-46; G. Latacz, E. Pekala, K. Kiec-Kononowicz, Biotechnologia 2006, 2, 189-205.

Further suitable hydantoinases are described by K. Yokozeki, H. Yoshiteru, K. Kubota, Agric. Biol. Chem. 1987, 51, 737-746.

The hydantoinase E₂ that may be used in preferred step (b) of the method according to the present invention may be a hydantoinase categorized in the EC class EC 3.5.2.2.

The following table 2 gives preferred examples for polypeptide sequences of hydantoinases E₂ that may be preferably used in step (b) of the preferred embodiment of the method according to the first aspect of the invention. The genes encoding the respective hydantoinase E₂ and the respective accession code are indicated as far as known.

TABLE 2
Hydantoinases (EC 3.5.2.2)

| | | GenBank/ | SEQ ID NO: |
|---|---|---|---|
| | | UniProt | of the |
| Strain | Gene name | accession | polypeptide |
| Arthrobacter | hyuH | | SEQ ID NO: 10 |
| aurescens DSM 9771 | | | |
| Pseudomonas | not assigned | S5MPT0 | SEQ ID NO: 11 |
| fluorescens | | | |
| Pseudomonas | dht | Q9I676 | SEQ ID NO: 12 |
| aeruginosa | | | |
| Bacillus fordii | not assigned | ABL14245 | SEQ ID NO: 13 |
| MH602 | | | |
| Arthrobacter | hyuH | AAL55412 | SEQ ID NO: 14 |
| sp. BT801 | | | |
| Alcaligenes faecalis | not assigned | | SEQ ID NO: 15 |
| subsp. faecalis | | | |
| Microbacterium | not assigned | | SEQ ID NO: 16 |
| liquefaciens | | | |
| strain AJ3912 | | | |

In a preferred embodiment of the method according to the first aspect of the present invention, the reaction according to step (b) is catalyzed by an hydantoinase E₂, wherein the polypeptide sequence of E₂ is selected from the group consisting of SEQ ID NO: 10 and variants thereof, SEQ ID NO: 11 and variants thereof, SEQ ID NO: 12 and variants thereof, SEQ ID NO: 13 and variants thereof, SEQ ID NO: 14 and variants thereof, SEQ ID NO: 15 and variants thereof, SEQ ID NO: 16 and variants thereof, preferably SEQ ID NO: 10 and variants thereof.

### 4.5.9 Assays D_{L} and do for Determining Hydantoinase Activity

The skilled person is aware of hydantoinases, in particular L-hydantoinases, that may be used in step (b) of the preferred method according to the first aspect of the invention.

In particular, Assay D_{L}, described in the following, may be used to determine hydantoinase and L-hydantoinase activity of a given enzyme E_{Y} and may advantageously be used according to the invention to determine hydantoinase and L-hydantoinase activity in variants of SEQ ID NO: 10, variants of SEQ ID NO: 11, variants of SEQ ID NO: 12, variants of SEQ ID NO: 13, variants of SEQ ID NO: 14, variants of SEQ ID NO: 15, variants of SEQ ID NO: 16.

For comparative reasons, Assay D_{D} may be used to determine D-hydantoinase activity of a given enzyme E_{Y}.

For the purpose of Assay D_{L} and Assay D_{D}, the molar mass of the enzyme E_{Y} to be tested is calculated as the molar mass of the polypeptide sequence of E_{Y}.

### 4.5.9.1 Assay D_{L}:

To 0.9 ml of an aqueous reaction solution (phosphate buffer, pH 7.2, 10 mM MnCl₂), containing 50 mM of an n-butyl P-ester of hydantoin glufosinate of the formula L-(III), wherein R═n-butyl, are added 400 nmol of E_{Y} in 0.1 ml aqueous phosphate buffer (pH 7.2, 10 mM MnCl₂). The resulting solution is incubated at 25° C., and the pH is held at pH 7.2 by addition of 1 M NaOH. After 300 minutes, the reaction is stopped by addition of 2 M HCl to achieve a pH of 2.5, and the molar amount of the n-butyl P-ester of carbamoyl glufosinate according to formula L-(II), wherein R═n-butyl, is determined. L-(II), wherein R═n-butyl, may be detected by the LC-MS method as described in the example section (item 5.4) for detection of LGA.

### 4.5.9.2 Assay D_{D}:

To 0.9 ml of an aqueous reaction solution (phosphate buffer, pH 7.2, 10 mM MnCl₂), containing 50 mM of an n-butyl P-ester of hydantoin glufosinate of the formula D-(III), wherein R═n-butyl, are added 400 nmol of E_{Y} in 0.1 ml aqueous phosphate buffer (pH 7.2, 10 mM MnCl₂). The resulting solution is incubated at 25° C., and the pH is held at pH 7.2 by addition of 1 M NaOH. After 300 minutes, the reaction is stopped by addition of 2 M HCl to achieve a pH of 2.5, and the molar amount of the n-butyl P-ester of carbamoyl glufosinate according to formula D-(II), wherein R═n-butyl, is determined. D-(II), wherein R═n-butyl, may be detected by the LC-MS method as described in the example section (item 5.4) for detection of LGA.

### 4.5.10 Assay E for Identifying Hydantoinases, L-Hydantoinases, D-Hydantoinases, L- and D-Enantiospecificity

The hydantoinase E₂ according to the invention is preferably an L-hydantoinase, more preferably L-enantiospecific.

Whether a given enzyme E_{Y} may be considered a hydantoinase E₂, in particular an L-hydantoinase, may be determined in the context of the present invention by the following Assay E:

### 4.5.10.1 Assay E:

E-1. Firstly, Assay D_{L} as set forth under item 4.5.9.1 is conducted, and the obtained molar amount of the compound of the formula L-(II), wherein R═n-butyl, is determined according to Assay D_{L}.

E-2 Secondly, Assay D_{D} as set forth under item 4.5.9.2 is conducted, and the obtained molar amount of the compound of the formula D-(II), wherein R═n-butyl, is determined according to Assay D_{D}.

E-3. Then, step E-1 is repeated, except that instead of the addition of 400 nmol E_{X} in 0.1 ml aqueous phosphate buffer (pH 7.2, 10 mM MnCl₂), 0.1 ml aqueous phosphate buffer (pH 7.2, 10 mM MnCl₂) without E_{Y} is added.

E-4 Then, step E-2 is repeated, except that instead of the addition of 400 nmol E_{X} in 0.1 ml aqueous phosphate buffer (pH 7.2, 10 mM MnCl₂), 0.1 ml aqueous phosphate buffer (pH 7.2, 10 mM MnCl₂) without E_{Y} is added.

### 4.5.10.2 Hydantoinase Activity:

If the molar amount of the compound of the formula L-(II), wherein R═n-butyl, that is determined in step E-1, is greater than the molar amount of the compound of formula L-(II), wherein R═n-butyl, that is determined in step E-3, then E_{X} is deemed to have hydantoinase activity, and hence may be considered a hydantoinase E₂ in the context of the invention.

### 4.5.10.3 L-Hydantoinase Activity

### 4.5.10.3.1 L-Hydantoinases

(i) If the molar amount of the compound of the formula L-(II), wherein R═n-butyl, that is determined in step E-1, is greater than the molar amount of the compound of the formula L-(II), wherein R═n-butyl, that is determined in step E-3, and
(ii) if the molar amount of the compound of the formula D-(II), wherein R═n-butyl, that is determined in step E-2, is greater than or the same as the molar amount of the compound of the formula D-(II), wherein R═n-butyl, that is determined in step E-4, and
(iii) if, in addition, the molar amount of the compound of the formula L-(II), wherein R═n-butyl, that is determined in step E-1 is greater than the molar amount of the compound of the formula D-(II), wherein R═n-butyl, that is determined in step E-2,
→ then E_{Y} is deemed to have L-hydantoinase activity, and hence may be considered an L-hydantoinase in the context of the invention. In this case, E_{Y} is deemed to be “L-enantioselective” in the context of the invention.

For the sake of clarity it is pointed out that condition (iii) is automatically fulfilled in those cases in which the molar amount of the compound of formula D-(II), wherein R═n-butyl, that is determined in step E-2 is the same as the molar amount of the compound of formula D-(II), wherein R═n-butyl, that is determined in step E-4.
(i) If the molar amount of the compound of formula L-(II), wherein R═n-butyl, that is determined in step E-1, is greater than the molar amount of the compound of formula L-(II), wherein R═n-butyl, that is determined in step E-3, and
(ii) if the molar amount of the compound of the formula D-(II), wherein R═n-butyl, that is determined in step E-2, is the same as the molar amount of the compound of the formula D-(II), wherein R═n-butyl, that is determined in step E-4, and
→ then E_{Y} is deemed to have L-hydantoinase activity, and hence may be considered a L-hydantoinase in the context of the invention. In this case, E_{Y} is deemed to be not only “L-enantioselective”, but also “L-enantiospecific” in the context of the invention.

For L-hydantoinases E_{Y} that are not L-enantiospecific, the L-enantioselectivity may then be quantified by dividing the molar amount of the compound of formula L-(II), wherein R═n-butyl, that is determined in E-1, by the molar amount of the compound of formula D-(II), wherein R═n-butyl, that is determined in E-2, and then multiplying the obtained value by 100, giving the L-enantioselectivity of E_{Y} in %.

### 4.5.10.3.2 D-Hydantoinases

(i) If the molar amount of the compound of formula L-(II), wherein R═n-butyl, that is determined in step E-1, is greater than or the same as the molar amount of the compound of formula L-(II), wherein R═n-butyl, that is determined in step E-3, and
(ii) if the molar amount of the compound of formula D-(II), wherein R═n-butyl, that is determined in step E-2 is greater than the molar amount of the compound of formula D-(II), wherein R═n-butyl, that is determined in step E-4, and
(iii) if, in addition, the molar amount of the compound of formula D-(II), wherein R═n-butyl, that is determined in step E-2, is greater than the molar amount of the compound of formula L-(II), wherein R═n-butyl, that is determined in step E-1,
→ then E_{Y} is deemed to have D-hydantoinase activity, and hence may be considered a D-hydantoinase. In this case, E_{Y} is “D-enantioselective” in the context of the invention.

For the sake of clarity it is pointed out that condition (iii) is automatically fulfilled in those cases in which the molar amount of the compound of formula L-(II), wherein R═n-butyl, that is determined in step E-1 is the same as the molar amount of the compound of formula L-(II), wherein R═n-butyl, that is determined in step E-3.
(i) If the molar amount of the compound of formula L-(II), wherein R═n-butyl, that is determined in step E-1, is the same as the molar amount of the compound of formula L-(II), wherein R═n-butyl, that is determined in step E-3, and
(ii) if the molar amount of the compound of formula D-(II), wherein R═n-butyl, that is determined in step E-2, is the greater than the molar amount of the compound of formula D-(II), wherein R═n-butyl, that is determined in step E-4,
→ then E_{X} is deemed to have D-hydantoinase activity, and hence may be considered a D-hydantoinase in the context of the invention. In this case, E_{Y} is deemed to be not only “D-enantioselective”, but also “D-enantiospecific” in the context of the invention.

For D-hydantoinases E_{Y} that are not D-enantiospecific, the D-enantioselectivity may then be quantified by dividing the molar amount of the compound of formula D-(II), wherein R═n-butyl, that is determined in step E-2, by the molar amount of the compound of formula L-(II), wherein R═n-butyl, that is determined in step E-1 and then multiplying the obtained value by 100, giving the D-enantioselectivity of E_{X} in %.

### 4.5.10.3.3 Non-Enantioselective Hydantoinases

(i) If the molar amount of the compound of formula L-(II), wherein R═n-butyl, that is determined in step E-1, is greater than the molar amount of the compound of formula L-(II), wherein R═n-butyl, that is determined in step E-3, and
(ii) if the molar amount of the compound of formula D-(II), wherein R═n-butyl, that is determined in step E-2, is greater than the molar amount of the compound of formula D-(II), wherein R═n-butyl, that is determined in step E-4, and
(iii) if, in addition, the molar amount of the compound of formula D-(II), wherein R═n-butyl, that is determined in step E-2 is the same as the molar amount of the compound of formula L-(II), wherein R═n-butyl, that is determined in step E-1,
→ then E_{Y} is deemed to be a non-enantioselective hydantoinase in the context of the invention. In this case, E_{Y} is “non-enantioselective”.

### 4.5.11 Assay F for Identifying Preferred Hydantoinases Variants of SEQ ID NOs: 10-16

### 4.5.11.1 L- and D-Enantioselective Hydantoinase Variants

An enzyme, the polpypetide sequence of which is selected from the group consisting of SEQ ID NO: 10, SEQ ID NO: 11, SEQ ID NO: 12, SEQ ID NO: 13, SEQ ID NO: 14, SEQ ID NO: 15, SEQ ID NO: 16 has hydantoinase, in particular L-hydantoinase activity.

In a preferred embodiment of the method according to the first aspect of the invention, the polypeptide sequence of the hydantoinase, in particular the L-hydantoinase E₂ is selected from the group consisting SEQ ID NO: 10 and variants thereof, SEQ ID NO: 11 and variants thereof, SEQ ID NO: 12 and variants thereof, SEQ ID NO: 13 and variants thereof, SEQ ID NO: 14, and variants thereof, SEQ ID NO: 15 and variants thereof, SEQ ID NO: 16 and variants thereof, more preferably SEQ ID NO: 10 and variants thereof.

The term variant is defined under item 4.3.

In the context of the invention, an enzyme, the polypeptide sequence of which is a variant of one of SEQ ID NO: 10, SEQ ID NO: 11, SEQ ID NO: 12, SEQ ID NO: 13, SEQ ID NO: 14, SEQ ID NO: 15, SEQ ID NO: 16, has hydantoinase activity, preferably L-hydantoinase activity, more preferably is L-enantiospecific.

Whether a given enzyme E_{Y}, the polypeptide sequence of which is a variant of one of SEQ ID NO: 10, SEQ ID NO: 11, SEQ ID NO: 12, SEQ ID NO: 13, SEQ ID NO: 14, SEQ ID NO: 15, SEQ ID NO: 16, has hydantoinase activity, L-hydantoinase activity and is L-enantiospecific may be determined as set forth under items 4.5.10.2 and 4.5.10.3.1, respectively.

The hydantoinase activity of a given hydantoinase E_{2V}, the polypeptide sequence of which is a variant of one of SEQ ID NO: 10, SEQ ID NO: 11, SEQ ID NO: 12, SEQ ID NO: 13, SEQ ID NO: 14, SEQ ID NO: 15, SEQ ID NO: 16, relative to the hydantoinase activity of an hydantoinase E_{2S}, wherein the polypeptide sequence of E_{2S} is selected from SEQ ID NO: 10, SEQ ID NO: 11, SEQ ID NO: 12, SEQ ID NO: 13, SEQ ID NO: 14, SEQ ID NO: 15, SEQ ID NO: 16, may be determined in the context of the present invention by the following Assay F:

### 4.5.11.2 Assay F

F-1 Assay D_{L} as set forth under item 4.5.9.1 is conducted, wherein E_{2S} is the enzyme to be tested. The obtained molar amount of the compound according to formula L-(II), wherein R═n-butyl, is determined according to Assay D_{L}.

F-2 Step F-1 is repeated, except that, instead of E_{2S}, E_{2V} is used as the enzyme to be tested. F-3. Then, the molar amount of the compound according to formula L-(II), wherein R═n-butyl, that is determined in step F-2, is divided by the molar amount of the compound according to formula

L-(II), wherein R═n-butyl, that is determined in step F-1, and the obtained ratio is multiplied by 100, giving the hydantoinase activity of hydantoinase E_{2V} relative to the hydantoinase activity of the hydantoinase E_{2S}, in %.

### 4.5.12 Preferred Hydantoinase Variants of SEQ ID NOs: 10-16

In the context of the invention, hydantoinases E₂, the polypeptide sequence of which is selected from the group consisting of SEQ ID NO: 10, SEQ ID NO: 11; SEQ ID NO: 12, SEQ ID NO: 13, SEQ ID NO: 14, SEQ ID NO: 15, SEQ ID NO: 16, are generally denoted as “E_{2S}”. Hydantoinases E₂, the polypeptide sequence of which is selected from variants of a sequence selected from the group consisting of SEQ ID NO: 10, SEQ ID NO: 11; SEQ ID NO: 12, SEQ ID NO: 13, SEQ ID NO: 14, SEQ ID NO: 15, SEQ ID NO: 16, are generally denoted as “E_{2V}”.

In a preferred embodiment of the method according to the first aspect of the present invention, the reaction in step (b) is catalyzed by a hydantoinase E₂, preferably an L-hydantoinase E₂, and the polypeptide sequence of the hydantoinase E₂, preferably the L-hydantoinase E₂, is selected from the group consisting of SEQ ID NO: 10 and variants thereof, SEQ ID NO: 11 and variants thereof, SEQ ID NO: 12 and variants thereof, SEQ ID NO: 13 and variants thereof, SEQ ID NO: 14 and variants thereof, SEQ ID NO: 15 and variants thereof, SEQ ID NO: 16 and variants thereof. More preferably, the reaction in step (b) is catalyzed by a hydantoinase E₂, preferably an

L-hydantoinase E₂, and the polypeptide sequence of the hydantoinase E₂, preferably the L-hydantoinase E₂, is selected from the group consisting of SEQ ID NO: 10 and variants of SEQ ID NO: 10.

### 4.5.12.1 Preferred Variants of SEQ ID NO: 10

According to the invention, the polypeptide sequence of the hydantoinase E₂, preferably the polypeptide sequence of the L-hydantoinase E₂, may also be a variant of SEQ ID NO: 10.

The hydantoinase E₂, the polypeptide sequence of which is SEQ ID NO: 10, is denoted as “E_{210S}”.

The hydantoinase E₂, the polypeptide sequence of which is selected from variants of SEQ ID NO: 10, are generally denoted as “E_{210V}”.

A variant of the polypeptide sequence of SEQ ID NO: 10 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 10.

The polypeptide sequence of a variant of the polypeptide sequence SEQ ID NO: 10 is not identical to SEQ ID NO: 10.

According to the invention, a hydantoinase E_{210V} has hydantoinase activity and preferably L-hydantoinase activity, determined as described under items 4.5.10.2 and 4.5.10.3.1.

According to the invention, a hydantoinase E_{210V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{210S}, wherein the hydantoinase activity of E_{210V}, relative to the hydantoinase activity of E_{210S} is determined by Assay F described under item 4.5.11.2.

It is even more preferably according to the invention, that a hydantoinase E_{210V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{210S}, wherein the hydantoinase activity of E_{210V}, relative to the hydantoinase activity E_{210S} is determined by Assay F described under item 4.5.11.2.

### 4.5.12.2 Preferred Variants of SEQ ID NO: 11

According to the invention, the polypeptide sequence of the hydantoinase E₂, preferably the polypeptide sequence of the L-hydantoinase E₂, may also be a variant of SEQ ID NO: 11.

The hydantoinase E₂, the polypeptide sequence of which is SEQ ID NO: 11, is denoted as “E_{211S}”. The hydantoinase E₂, the polypeptide sequence of which is selected from variants of SEQ ID NO: 11, are generally denoted as “E_{211V}”.

A variant of the polypeptide sequence of SEQ ID NO: 11 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 11.

The polypeptide sequence of a variant of the polypeptide sequence SEQ ID NO: 11 is not identical to SEQ ID NO: 11.

According to the invention, a hydantoinase E_{211V} has hydantoinase activity and preferably L-hydantoinase activity, determined as described under items 4.5.10.2 and 4.5.10.3.1.

According to the invention, a hydantoinase E_{211V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{211S}, wherein the hydantoinase activity of E_{211V}, relative to the hydantoinase activity of E_{211S} is determined by Assay F described under item 4.5.11.2.

It is even more preferably according to the invention, that a hydantoinase E_{211V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{211S}, wherein the hydantoinase activity of E_{211V}, relative to the hydantoinase activity E_{211S} is determined by Assay F described under item 4.5.11.2.

### 4.5.12.3 Preferred Variants of SEQ ID NO: 12

According to the invention, the polypeptide sequence of the hydantoinase E₂, preferably the polypeptide sequence of the L-hydantoinase E₂, may also be a variant of SEQ ID NO: 12.

The hydantoinase E₂, the polypeptide sequence of which is SEQ ID NO: 12, is denoted as “E_{212S}”. The hydantoinase E₂, the polypeptide sequence of which is selected from variants of SEQ ID NO: 12, are generally denoted as “E_{212V}”.

A variant of the polypeptide sequence of SEQ ID NO: 12 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 12.

The polypeptide sequence of a variant of the polypeptide sequence SEQ ID NO: 12 is not identical to SEQ ID NO: 12.

According to the invention, a hydantoinase E_{212V} has hydantoinase activity and preferably L-hydantoinase activity, determined as described under items 4.5.10.2 and 4.5.10.3.1.

According to the invention, a hydantoinase E_{212V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{212S}, wherein the hydantoinase activity of E_{212V}, relative to the hydantoinase activity of E_{212S} is determined by Assay F described under item 4.5.11.2.

It is even more preferably according to the invention, that a hydantoinase E_{212V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{212S}, wherein the hydantoinase activity of E_{212V}, relative to the hydantoinase activity E_{212S} is determined by Assay F described under item 4.5.11.2.

### 4.5.12.4 Preferred Variants of SEQ ID NO: 13

According to the invention, the polypeptide sequence of the hydantoinase E₂, preferably the polypeptide sequence of the L-hydantoinase E₂, may also be a variant of SEQ ID NO: 13.

The hydantoinase E₂, the polypeptide sequence of which is SEQ ID NO: 13, is denoted as “E_{213S}”. The hydantoinase E₂, the polypeptide sequence of which is selected from variants of SEQ ID NO: 13, are generally denoted as “E_{213V}”.

A variant of the polypeptide sequence of SEQ ID NO: 13 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 13.

The polypeptide sequence of a variant of the polypeptide sequence SEQ ID NO: 13 is not identical to SEQ ID NO: 13.

According to the invention, a hydantoinase E_{213V} has hydantoinase activity and preferably L-hydantoinase activity, determined as described under items 4.5.10.2 and 4.5.10.3.1.

According to the invention, a hydantoinase E_{213V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{213S}, wherein the hydantoinase activity of E_{213V}, relative to the hydantoinase activity of E_{213S} is determined by Assay F described under item 4.5.11.2.

It is even more preferably according to the invention, that a hydantoinase E_{213V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{213S}, wherein the hydantoinase activity of E_{213V}, relative to the hydantoinase activity E_{213S} is determined by Assay F described under item 4.5.11.2.

### 4.5.12.5 Preferred Variants of SEQ ID NO: 14

According to the invention, the polypeptide sequence of the hydantoinase E₂, preferably the polypeptide sequence of the L-hydantoinase E₂, may also be a variant of SEQ ID NO: 14.

The hydantoinase E₂, the polypeptide sequence of which is SEQ ID NO: 14, is denoted as “E_{214S}”. The hydantoinase E₂, the polypeptide sequence of which is selected from variants of SEQ ID NO: 14, are generally denoted as “E_{214V}”.

A variant of the polypeptide sequence of SEQ ID NO: 14 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably >93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 14.

The polypeptide sequence of a variant of the polypeptide sequence SEQ ID NO: 14 is not identical to SEQ ID NO: 14.

According to the invention, a hydantoinase E_{214V} has hydantoinase activity and preferably L-hydantoinase activity, determined as described under items 4.5.10.2 and 4.5.10.3.1.

According to the invention, a hydantoinase E_{214V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{214S}, wherein the hydantoinase activity of E_{214V}, relative to the hydantoinase activity of E_{214S} is determined by Assay F described under item 4.5.11.2.

It is even more preferably according to the invention, that a hydantoinase E_{214V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{214S}, wherein the hydantoinase activity of E_{214V}, relative to the hydantoinase activity E_{214S} is determined by Assay F described under item 4.5.11.2.

### 4.5.12.6 Preferred Variants of SEQ ID NO: 15

According to the invention, the polypeptide sequence of the hydantoinase E₂, preferably the polypeptide sequence of the L-hydantoinase E₂, may also be a variant of SEQ ID NO: 15.

The hydantoinase E₂, the polypeptide sequence of which is SEQ ID NO: 15, is denoted as “E_{215S}”. The hydantoinase E₂, the polypeptide sequence of which is selected from variants of SEQ ID NO: 15, are generally denoted as “E_{215V}”.

A variant of the polypeptide sequence of SEQ ID NO: 15 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 15.

The polypeptide sequence of a variant of the polypeptide sequence SEQ ID NO: 15 is not identical to SEQ ID NO: 15.

According to the invention, a hydantoinase E_{215V} has hydantoinase activity and preferably L-hydantoinase activity, determined as described under items 4.5.10.2 and 4.5.10.3.1.

According to the invention, a hydantoinase E_{215V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{215S}, wherein the hydantoinase activity of E_{215V}, relative to the hydantoinase activity of E_{215S} is determined by Assay F described under item 4.5.11.2.

It is even more preferably according to the invention, that a hydantoinase E_{215V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{215S}, wherein the hydantoinase activity of E_{215V}, relative to the hydantoinase activity E_{215S} is determined by Assay F described under item 4.5.11.2.

### 4.5.12.7 Preferred Variants of SEQ ID NO: 16

According to the invention, the polypeptide sequence of the hydantoinase E₂, preferably the polypeptide sequence of the L-hydantoinase E₂, may also be a variant of SEQ ID NO: 16.

The hydantoinase E₂, the polypeptide sequence of which is SEQ ID NO: 16, is denoted as “E_{216S}”. The hydantoinase E₂, the polypeptide sequence of which is selected from variants of SEQ ID NO: 16, are generally denoted as “E_{216V}”.

A variant of the polypeptide sequence of SEQ ID NO: 16 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 16.

The polypeptide sequence of a variant of the polypeptide sequence SEQ ID NO: 16 is not identical to SEQ ID NO: 16.

According to the invention, a hydantoinase E_{216V} has hydantoinase activity and preferably L-hydantoinase activity, determined as described under items 4.5.10.2 and 4.5.10.3.1.

According to the invention, a hydantoinase E_{216V} preferably has hydantoinase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoinase activity of the hydantoinase E_{216S}, wherein the hydantoinase activity of E_{216V}, relative to the hydantoinase activity of E_{216S} is determined by Assay F described under item 4.5.11.2.

It is even more preferably according to the invention, that a hydantoinase E_{216V} has hydantoinase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoinase activity of the hydantoinase E_{216S}, wherein the hydantoinase activity of E_{216V}, relative to the hydantoinase activity E_{216S} is determined by Assay F described under item 4.5.11.2.

### 4.5.13 Preferred Method Conditions in Step (b)

The reaction in step (b) of the method according to the first aspect of the present invention may be carried out under conditions known to the skilled person.

The reaction medium is preferably aqueous, more preferably an aqueous buffer.

Exemplary buffers commonly used in biotransformation reactions and advantageously used herein include Tris, phosphate, or any of Good's buffers, such as 2-(N-morpholino) ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”).

In some embodiments, ammonium can act as a buffer. One or more organic solvents can also be added to the reaction.

The buffer preferably contains metal salts, more preferably metal salts such as halogenides of metals, preferably halogenides of monovalent or bivalent or trivalent metals, preferably chlorides of monovalent or bivalent metals, preferably CoCl₂ or MnCl₂, preferably CoCl₂.

The concentration of these metal salts in the reaction medium is preferably in the range from 1 μM to 1 M, more preferably 1 mM to 100 mM, even more preferably 1 to 10 mM.

Preferably, step (b) of the method according to the first aspect of the invention is carried out in a phosphate buffer.

The pH of the reaction medium in step (b) of the method according to the first aspect of the invention is preferably in the range of from 2 to 10, more preferably in the range of from 5 to 8, more preferably 7.2 to 7.5, most preferably 7.5.

Preferably, step (b) of the method according to the first aspect of the invention is carried out at a temperature in the range of from 20° C. to 70° C., more preferably in the range of from 30° C. to 55° C., most preferably 50° C.

Preferably, the total concentration of all hydantoinases E₂ in the reaction solution in step (b) is in the range of from 1 μM to 10 mM, preferably 10 μM to 1 mM, more preferably 0.1 mM to 0.5 mM, most preferably 0.4 mM.

In alternative preferred embodiments, the total concentration of all hydantoinases E₂ in the reaction solution in step (b) is in the range of from 1 μg/l to 10 g/l, preferably 0.1 mg/l to 5 g/l, more preferably 1 mg/l to 1 g/l, more preferably 5 mg/l to 500 mg/l.

Preferably, the initial concentration of all the compounds according to formula L-(III) in the reaction medium in step (b) is in the range of from 1 μM to 1 M, preferably of from 10 μM to 0.5 M, more preferably of from 0.1 mM to 0.1 M, more preferably of from 1 mM to 10 mM, most preferably 1.25 mM.

If compounds according to formula D-(III) are present in the reaction medium in step (b), the initial concentration of all the compounds according to formula D-(III) in the reaction medium is preferably from 1% to 100% the concentration of all the compounds according to formula L-(III), more preferably 10% to 100% the concentration, even more preferably 50 to 100%, even more preferably 100% the concentration of all the compounds according to formula L-(III).

Preferably, step (b) is carried out in the same reaction medium in which step (c) is carried out. In this case, preferably, the initial concentration of all the compounds according to formula L-(III) in the reaction medium in step (b) is in the range of from 1 μM to 1 M, preferably of from 10 μM to 0.5 M, more preferably of from 0.1 mM to 0.1 M, more preferably of from 1 mM to 10 mM, most preferably 1.25 mM.

“Initial concentration of all the compounds according to formula L-(III)/D-(III)” refers to the concentration of the respective compound L-(III) or D-(III), respectively, in the reaction medium when the respective compounds are employed in step (b).

### 4.5.14 Step (a)

In a preferred embodiment of the method according to the first aspect of the invention, the compound according to formula L-(III) is obtained by a step (a) in which a compound according to formula D-(III) is reacted to give a compound according to formula L-(III):

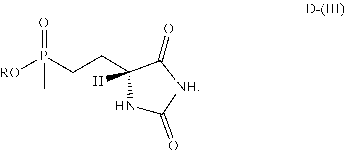

Step (a) gives the starting material for step (b), and R in D-(III) has the same meaning as described for L-(III) [or L-(I)].

The reaction according to step (a) may be carried out enzymatically or non-enzymatically, preferably enzymatically. More preferably, the reaction according to step (a) is catalyzed by a hydantoin racemase E₃.

In preferred step (a), the compound according to formula D-(III) is employed in step (a) as a mixture M_{III} of D-(III) with its enantiomer L-(III).

In a preferred embodiment, the mixture M_{II} is a racemic mixture of enantiomer L-(III) and enantiomer D-(III), meaning that the molar ratio of enantiomer L-(III) to enantiomer D-(III) is essentially 1:1.

In other preferred embodiments, the molar ratio of enantiomer L-(III) to enantiomer D-(III) in mixture M_{III} is in the range of from 3:2 to 1:99, more preferably in the range of from 1.01:1 to 1:99, more preferably in the range of from 1:1 to 1:99, more preferably in the range of from 1:1.01 to 1:99, more preferably in the range of from 1:1.01 to 1:9, more preferably in the range of from 1:1.01 to 1:8, more preferably in the range of from 1:1.01 to 1:3.

Alternatively, enantiomer D-(III) is comprised in an excess to L-(III) in mixture M_{III}, meaning that, while L-(III) is present in the mixture M_{III}, the molar ratio of enantiomer L-(III) to enantiomer D-(III) in mixture M_{III} is <1:1, preferably <0.9:1, more preferably <0.75:1, more preferably <0.5:1, more preferably <0.2:1, more preferably <0.1:1, more preferably <0.01:1.

4.5.14.1 Step (a) without Enzymatic Catalysis

Step (a) may be carried out non-enzymatically, i.e. without the use of an enzyme. The reaction of compounds according to step D-(III) to compounds according to L-(III) proceeds in alkaline solution, as known to the skilled person and as described by Slomka et al., M. Bovarnick & H. T. Clarke, Journal of the American Chemical Society 1938, 60, 2426-2430, by R. A. Lazarus, J. Org. Chem. 1990, 55, 4755-4757, and by A. S. Bommarius, M. Kottenhahn, H. Klenk, K. Drauz: “A direct route from hydantoins to D-amino acids employing a resting cell biocatalyst with D-hydantoinase and D-carbamoylase activity” on page 164 and 167 in “Microbial Reagents in Organic Synthesis” Series C: Mathematical and Physical Sciences-Vol. 381, S. Servi (Ed.), 1992, Springer Science+Business Media, B. V., Dordrecht.

Therefore, if step (a) is carried out non-enzymatically, the conditions that are preferably applied in the reaction medium in which non-enzymatic step (a) is carried out are preferably those that are described for the preferred conditions for step (b) (item 4.5.13), except that the pH is ≥8, preferably in the range of 8 to 12, more preferably 8 to 11, more preferably 8 to 10, even more preferably 8 to 9. As in these embodiments, the preferred conditions in step (a) and (b) with respect to the pH ranges are different, it is preferable that the reaction media in steps (a) and (b) are different.

4.5.14.2 Enzymatic Conversion of D-(III) into L-(III)

Step (a) is preferably carried out enzymatically, i.e. the reaction according to step (a) is preferably catalyzed by a hydantoin racemase E₃.

Namely, it was surprisingly found that hydantoin racemases accept compounds of formula D-(III) as substrates and convert them to products according to formulae L-(III), and hence catalyze the reaction according to step (a), while there is no catalysis of the corresponding compound according to formula D-(III), in which R═H.

This finding is of high scientific and economic value, as it further expands the scope of new starting materials for the production of L-glufosinate alkyl esters and L-glufosinate via new synthetic routes. Moreover, this finding also opens new possibilities of enantioselective production of LGA from racemic mixtures of L-(III) and D-(III), as embodied in the method according to the second aspect o the invention.

In nature, hydantoin racemases catalyze the conversion of one of the two hydantoin enantiomers H_{L} and H_{R} into the other (see the following reaction <3>):

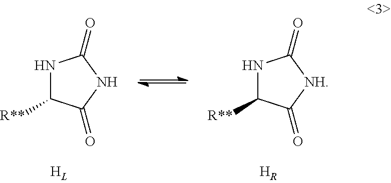

It was now surprisingly found that hydantoin racemases also accept substrates in which

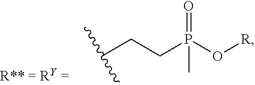

wherein R has the above meaning and wherein preferably

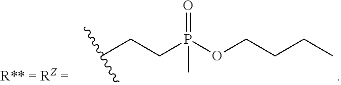 [SMILES: *CCP(C)(=O)OCCCC]

Surprisingly, they do not accept substrates in which

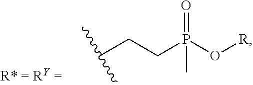

wherein R═H.

Suitable hydantoin racemases are described e.g. in WO 01/23582 A1 and by U. Engel, J. Rudat, C. Syldatk in “The hydantoinase process: recent developments for the production of non-canonical amino acids” in the book “Industrial biocatalysis” by P. Grunwald (Ed.), Pan Stanford Series on Biocatalysis, 2015, pages 817-862, and by F. J. Las Heras-Vazquez, J. M. Clemente-Jimenez, S. Martinez-Rodriguez, F. Rodriguez-Vico in “Hydantoin racemase: the key enzyme for the production of optically pure a-amino acids” in chapter 12 of the book “Modern Biocatalysis: Stereoselective and environmentally friendly reactions” by W. Fessner, T. Anthonsen (Eds), Weinheim: WILEY-VCH Verlag Gmbh & Co, 2009, pages 173-193.

A hydantoin racemase E₃ that may be used in optional step (a) of the method according to the first aspect of the invention may originate from Agrobacterium S p., in particular Agrobacterium S train IP_I-671; Arthrobacter S p., in particular Arthrobacter aurescens, more in particular Arthrobacter aurescens DSM 3745 or Arthrobacter sp. BT801; Flavobacterium S p., in particular Flavobacterium S p. AJ 11199; Microbacterium S p., in particular Microbacterium liquefaciens, preferably Microbacterium liquefaciens AJ 3912; Pasteurella S p., in particular Pasteurella S p. AJ11221; Pseudomonas S p., in particular Pseudomonas S p. NS671; Pyrococcus S p., in particular Pyrococcus horikoshii OT3; Rhodococcus S p., in particular Rhodococcus R04; Sinorhizobium S p., in particular Sinorhizobium meliloti, more in particular Sinorhizobium meliloti CECT 4114, most preferably from Arthrobacter aurescens DSM 3745.

A hydantoin racemase E₃ suitable for the method according to the present invention may be the enzyme HyuR, which originates from Arthrobacter aurescens DSM 3745. Another enzyme may be selected from HyuE, Hyu2, HRase, HyuA, PH1054.

The hydantoin racemase E₃ that may preferably be used in preferred step (a) of the method according to the first aspect of the invention may be categorized in the EC class 5.1.99.5.

The following table 3 gives preferred examples for polypeptide sequences of hydantoin racemase E₃ that may be preferably used in step (a) of the method according to the first aspect of the invention. The genes encoding the respective hydantoin racemase E₃ and the respective accession code are indicated as far as known.

TABLE 3
Hydantoin Racemases (EC 5.1.99.5)

| | | GenBank/ | SEQ ID NO: |
|---|---|---|---|
| | Gene | UniProt | of the |
| Strain | name | accession | polypeptide |
| Arthrobacter | hyuR | | SEQ ID NO: 17 |
| aurescens DSM 3745 | | | |
| Pseudomonas | hyuE | Q00924 | SEQ ID NO: 18 |
| sp. NS671 | | | |
| Rhodococcus R04 | hyu2 | | SEQ ID NO: 19 |
| Microbacterium | “HRase” | | SEQ ID NO: 20 |
| liquefaciens AJ 3912 | | | |
| Sinorhizobium meliloti | hyuA | Q6TMG4 | SEQ ID NO: 21 |
| CECT 4114 | | | |
| Flavobacterium | “HRase” | | SEQ ID NO: 22 |
| sp. AJ11199 | | | |
| Agrobacterium | hyuA | | SEQ ID NO: 23 |
| IP_I-671 | | | |
| Pasteurella | not assigned | | SEQ ID NO: 24 |
| sp. AJ 11221 | | | |
| Pyrococcus | PH1054 | O58781 | SEQ ID NO: 25 |
| horikoshii OT3 | | | |
| Arthrobacter | hyuA | AAL55411 | SEQ ID NO: 26 |
| sp. BT801 | | | |

In a preferred embodiment of the preferred method according to the second aspect of the present invention, the reaction according to step (a) is catalyzed by a hydantoin racemase E₃, wherein the polypeptide sequence of E₃ is selected from the group consisting of SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, SEQ ID NO: 26 and variants thereof.

### 4.5.14.3 Assay G for Determining Hydantoin Racemase Activity

The skilled person is aware of hydantoin racemases, that may be used in preferred step (a) of the method according to the first aspect of the invention.

In particular, Assay G, described in the following, may be used to determine hydantoin racemase activity of a given enzyme E_{Z} and may advantageously be used according to the invention to determine carbamoylase and L-carbamoylase activity in variants of SEQ ID NO: 17, variants of variants of SEQ ID NO: 18, variants of SEQ ID NO: 19, variants of SEQ ID NO: 20, variants of SEQ ID NO: 21, variants of SEQ ID NO: 22, variants of variants of SEQ ID NO: 23, variants of SEQ ID NO: 24, variants of SEQ ID NO: 25, variants of SEQ ID NO: 26.

For the purpose of Assay G, the molar mass of the enzyme E_{Z} to be tested is calculated as the molar mass of the polypeptide sequence of E_{Z}.

### Assay G:

To 0.9 ml of an aqueous reaction solution (phosphate buffer, pH 7.2, 10 mM Mg₂Cl), containing 50 mM of the pure D-enantiomer of an n-butyl ester of hydantoin glufosinate of the formula D-(III), wherein R═n-butyl, are added 400 nmol of E_{Z} in 0.1 ml aqueous phosphate buffer (pH 7.2, 10 mM MnCl₂).

The resulting solution is incubated at 25° C., and the pH is held at pH 7.2 by addition of 0.5 M. After 300 minutes, the reaction is stopped by addition of 2 M HCl to achieve a pH of 2.5. The molar amount of the L-enantiomer of formula L-(III), wherein R═n-butyl, is measured, at least every 3 minutes (determination by LC-MS, e.g. by the LC-MS method described in the example section, item 5.4, for the detection of LGA).

### 4.5.14.4 Assay H for Identifiying Hydantoin Racemases

Whether a given enzyme E_{Z} may be considered a hydantoin racemase E₃, may be determined in the context of the present invention by the following Assay H:

H-1 Firstly, Assay G as set forth under item 4.5.14.3 is conducted, and the obtained molar amount of L-(III) is determined according to Assay G.

H-2 Then, step H-1 is repeated, except that instead of the addition of 400 nmol E_{Z} in 0.1 ml aqueous phosphate buffer (pH 7.2, 10 mM MnCl₂), 0.1 ml aqueous phosphate buffer (pH 7.2, 10 mM MnCl₂) without E_{Z} is added.

### 4.5.14.5 Hydantoin Racemase Activity

If the molar amount of the compound of formula L-(III), wherein R═n-butyl, that is determined in step H-1 is greater than the molar amount of the compound of formula L-(III), wherein R═n-butyl, that is determined in step H-2, then E_{Z} is deemed to have hydantoin racemase activity, and hence may be considered a hydantoin racemase E₃ in the context of the invention.

### 4.5.14.6 Assay J for Identifying Preferred Hydantoin Racemase Variants of SEQ ID NO: 17-26

An enzyme, the polpypetide sequence of which is selected from the group consisting of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, has hydantoin racemase activity.

In a preferred embodiment of the method according to the first aspect of the invention, the polypeptide sequence of the hydantoin racemase E₃ is selected from the group consisting of SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, SEQ ID NO: 26 and variants thereof.

The term “variant” is defined under item 4.3.

In the context of the invention, an enzyme, the polypeptide sequence of which is a variant of one of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, has hydantoin racemase activity.

Whether a given enzyme E_{Z}, the polypeptide sequence of which is a variant of one SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, has hydantoin racemase activity may be determined as set forth under items 4.5.14.4 and 4.5.14.5.

The hydantoin racemase activity of a given hydantoin racemase E_{3V}, the polypeptide sequence of which is a variant of one of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, relative to the hydantoin racemase activity of a hydantoin racemase E_{3S}, wherein the polypeptide sequence of E_{3S} is selected from SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, may be quantified in the context of the present invention by the following Assay J.

### Assay J:

J-1 Firstly, Assay G as set forth under item 4.5.14.3 is conducted, wherein E_{3S} is the enzyme to be tested. The molar amount of the compound according to formula L-(III), wherein R═n-butyl, is determined according to Assay G.

J-2 Step J-1 is repeated, except that, instead of E_{3S}, E_{3V} is used as the enzyme to be tested.

J-3. Then, the molar amount of the compound according to formula L-(III), wherein R═n-butyl, is determined in step J-2, is divided by the molar amount of the compound according to formula L-(III), wherein R═n-butyl, is determined in step J-1, and the obtained ratio is multiplied by 100, giving the hydantoin racemase activity of hydantoin racemase E_{3V}, relative to the hydantoin racemase activity of the hydantoin racemase E_{3S}, in %.

### 4.5.14.7 Preferred Hydantoin Racemase Variants SEQ ID NO: 17-26

In the context of the present invention, hydantoin racemase E₃, the polypeptide sequence of which is selected from the group consisting of SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, are generally denoted as “E_{3S}”.

Hydantoin racemases E₃, the polypeptide sequence of which is selected from variants of a sequence selected from the group consisting of f SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, are generally denoted as “E_{3V}”.

In a preferred embodiment of the method according to the first aspect of the present invention, the reaction in step (a) is catalyzed by a hydantoin racemase E₃, and the polypeptide sequence of the hydantoin racemase E₃ is selected from the group consisting of SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, SEQ ID NO: 26 and variants thereof. More preferably, the reaction in step (a) is catalyzed by a hydantoin racemase E₃, and the polypeptide sequence of the hydantoin racemase E₃ is selected from the group consisting of SEQ ID NO: 1 and variants of SEQ ID NO: 1.

### 4.5.14.7.1 Preferred Variants of SEQ ID NO: 17

According to the invention, the polypeptide sequence of the hydantoin racemase E₃ may also be a variant of SEQ ID NO: 17.

The hydantoin racemase E₃, the polypeptide sequence of which is SEQ ID NO: 17, is denoted as “E_{317S}”. Hydantoin racemases E₃, the polypeptide sequence of which is is selected from variants of SEQ ID NO: 17, are generally denoted as “E_{317V}”.

A variant of the polypeptide sequence of SEQ ID NO: 17 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 17.

The polypeptide sequence of a variant of the polypeptide sequence SEQ ID NO: 17 is not identical to SEQ ID NO: 17.

According to the invention, a hydantoin racemase E_{317V} has hydantoin racemase activity, determined as described under item 4.5.14.4 and 4.5.14.5.

According to the invention, a hydantoin racemase E_{317V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{317S}, wherein the hydantoin racemase activity of E_{317V}, relative to the hydantoin racemase activity E_{317S} is determined by Assay J described under item 4.5.14.6.

It is even more preferably according to the invention, that a hydantoin racemase E_{317V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{317S}, wherein the hydantoin racemase activity of E_{317V}, relative to the hydantoin racemase activity of E_{317S} is determined by Assay J described under item 4.5.14.6.

### 4.5.14.7.2 Preferred Variants of SEQ ID NO: 18

According to the invention, the polypeptide sequence of the hydantoin racemase E₃ may also be a variant of SEQ ID NO: 18.

The hydantoin racemase E₃, the polypeptide sequence of which is SEQ ID NO: 18, is denoted as “E_{318S}”. Hydantoin racemases E₃, the polypeptide sequence of which is selected from variants of SEQ ID NO: 18, are generally denoted as “E_{318V}”.

A variant of the polypeptide sequence of SEQ ID NO: 18 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 18.

The polypeptide sequence of a variant of the polypeptide sequence SEQ ID NO: 18 is not identical to SEQ ID NO: 18.

According to the invention, a hydantoin racemase E_{318V} has hydantoin racemase activity, determined as described under item 4.5.14.4 and 4.5.14.5.

According to the invention, a hydantoin racemase E_{318V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{318S}, wherein the hydantoin racemase activity of E_{318V}, relative to the hydantoin racemase activity E_{318S} is determined by Assay J described under item 4.5.14.6.

It is even more preferably according to the invention, that a hydantoin racemase E_{318V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{318S}, wherein the hydantoin racemase activity of E_{318V}, relative to the hydantoin racemase activity of E_{318S} is determined by Assay J described under item 4.5.14.6.

### 4.5.14.7.3 Preferred Variants of SEQ ID NO: 19

According to the invention, the polypeptide sequence of the hydantoin racemase E₃ may also be a variant of SEQ ID NO: 19.

The hydantoin racemase E₃, the polypeptide sequence of which is SEQ ID NO: 19, is denoted as “E_{319S}”. Hydantoin racemases E₃, the polypeptide sequence of which is selected from variants of SEQ ID NO: 19, are generally denoted as “E_{319V}”.

A variant of the polypeptide sequence of SEQ ID NO: 19 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 19.

The polypeptide sequence of a variant of the polypeptide sequence SEQ ID NO: 19 is not identical to SEQ ID NO: 19.

According to the invention, a hydantoin racemase E_{319V} has hydantoin racemase activity, determined as described under item 4.5.14.4 and 4.5.14.5.

According to the invention, a hydantoin racemase E_{319V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{319S}, wherein the hydantoin racemase activity of E_{319V}, relative to the hydantoin racemase activity E_{319S} is determined by Assay J described under item 4.5.14.6.

It is even more preferably according to the invention, that a hydantoin racemase E_{319V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{319S}, wherein the hydantoin racemase activity of E_{319V}, relative to the hydantoin racemase activity of E_{319S} is determined by Assay J described under item 4.5.14.6.

### 4.5.14.7.4 Preferred Variants of SEQ ID NO: 20

According to the invention, the polypeptide sequence of the hydantoin racemase E₃ may also be a variant of SEQ ID NO: 20.

The hydantoin racemase E₃, the polypeptide sequence of which is SEQ ID NO: 20, is denoted as “E_{320S}”. Hydantoin racemases E₃, the polypeptide sequence of which is selected from variants of SEQ ID NO: 20, are generally denoted as “E_{320V}”.

A variant of the polypeptide sequence of SEQ ID NO: 20 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 20.

The polypeptide sequence of a variant of the polypeptide sequence SEQ ID NO: 20 is not identical to SEQ ID NO: 20.

According to the invention, a hydantoin racemase E_{320V} has hydantoin racemase activity, determined as described under item 4.5.14.4 and 4.5.14.5.

According to the invention, a hydantoin racemase E_{320V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{320S}, wherein the hydantoin racemase activity of E_{320V}, relative to the hydantoin racemase activity E_{320S} is determined by Assay J described under item 4.5.14.6.

It is even more preferably according to the invention, that a hydantoin racemase E_{320V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{320S}, wherein the hydantoin racemase activity of E_{320V}, relative to the hydantoin racemase activity of E_{320S} is determined by Assay J described under item 4.5.14.6.

### 4.5.14.7.5 Preferred Variants of SEQ ID NO: 21

According to the invention, the polypeptide sequence of the hydantoin racemase E₃ may also be a variant of SEQ ID NO: 21.

The hydantoin racemase E₃, the polypeptide sequence of which is SEQ ID NO: 21, is denoted as “E_{321S}”. Hydantoin racemases E₃, the polypeptide sequence of which is selected from variants of SEQ ID NO: 21, are generally denoted as “E_{321V}”.

A variant of the polypeptide sequence of SEQ ID NO: 21 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 21.

The polypeptide sequence of a variant of the polypeptide sequence SEQ ID NO: 21 is not identical to SEQ ID NO: 21.

According to the invention, a hydantoin racemase E_{321V} has hydantoin racemase activity, determined as described under item 4.5.14.4 and 4.5.14.5.

According to the invention, a hydantoin racemase E_{321V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{321S}, wherein the hydantoin racemase activity of E_{321V}, relative to the hydantoin racemase activity E_{321S} is determined by Assay J described under item 4.5.14.6.

It is even more preferably according to the invention, that a hydantoin racemase E_{321V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{321S}, wherein the hydantoin racemase activity of E_{321V}, relative to the hydantoin racemase activity of E_{321S} is determined by Assay J described under item 4.5.14.6.

### 4.5.14.7.6 Preferred Variants of SEQ ID NO: 22

According to the invention, the polypeptide sequence of the hydantoin racemase E₃ may also be a variant of SEQ ID NO: 22.

The hydantoin racemase E₃, the polypeptide sequence of which is SEQ ID NO: 22, is denoted as “E_{322S}”. Hydantoin racemases E₃, the polypeptide sequence of which is selected from variants of SEQ ID NO: 22, are generally denoted as “E_{322V}”.

A variant of the polypeptide sequence of SEQ ID NO: 22 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 22.

The polypeptide sequence of a variant of the polypeptide sequence SEQ ID NO: 22 is not identical to SEQ ID NO: 22.

According to the invention, a hydantoin racemase E_{322V} has hydantoin racemase activity, determined as described under item 4.5.14.4 and 4.5.14.5.

According to the invention, a hydantoin racemase E_{322V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{322S}, wherein the hydantoin racemase activity of E_{322V}, relative to the hydantoin racemase activity E_{322S} is determined by Assay J described under item 4.5.14.6.

It is even more preferably according to the invention, that a hydantoin racemase E_{322V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{322S}, wherein the hydantoin racemase activity of E_{322V}, relative to the hydantoin racemase activity of E_{322S} is determined by Assay J described under item 4.5.14.6.

### 4.5.14.7.7 Preferred Variants of SEQ ID NO: 23

According to the invention, the polypeptide sequence of the hydantoin racemase E₃ may also be a variant of SEQ ID NO: 23.

The hydantoin racemase E₃, the polypeptide sequence of which is SEQ ID NO: 23, is denoted as “E_{323S}”. Hydantoin racemases E₃, the polypeptide sequence of which is is selected from variants of SEQ ID NO: 23, are generally denoted as “E_{323V}”.

A variant of the polypeptide sequence of SEQ ID NO: 23 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 23.

The polypeptide sequence of a variant of the polypeptide sequence SEQ ID NO: 23 is not identical to SEQ ID NO: 23.

According to the invention, a hydantoin racemase E_{323V} has hydantoin racemase activity, determined as described under item 4.5.14.4 and 4.5.14.5.

According to the invention, a hydantoin racemase E_{323V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{323S}, wherein the hydantoin racemase activity of E_{323V}, relative to the hydantoin racemase activity E_{323S} is determined by Assay J described under item 4.5.14.6.

It is even more preferably according to the invention, that a hydantoin racemase E_{323V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{323S}, wherein the hydantoin racemase activity of E_{323V}, relative to the hydantoin racemase activity of E_{323S} is determined by Assay J described under item 4.5.14.6.

### 4.5.14.7.8 Preferred Variants of SEQ ID NO: 24

According to the invention, the polypeptide sequence of the hydantoin racemase E₃ may also be a variant of SEQ ID NO: 24.

The hydantoin racemase E₃, the polypeptide sequence of which is SEQ ID NO: 24, is denoted as “E_{324S}”. Hydantoin racemases E₃, the polypeptide sequence of which is selected from variants of SEQ ID NO: 24, are generally denoted as “E_{324V}”.

A variant of the polypeptide sequence of SEQ ID NO: 24 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 24.

The polypeptide sequence of a variant of the polypeptide sequence SEQ ID NO: 24 is not identical to SEQ ID NO: 24.

According to the invention, a hydantoin racemase E_{324V} has hydantoin racemase activity, determined as described under item 4.5.14.4 and 4.5.14.5.

According to the invention, a hydantoin racemase E_{324V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{324S}, wherein the hydantoin racemase activity of E_{324V}, relative to the hydantoin racemase activity E_{324S} is determined by Assay J described under item 4.5.14.6.

It is even more preferably according to the invention, that a hydantoin racemase E_{324V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{324S}, wherein the hydantoin racemase activity of E_{324V}, relative to the hydantoin racemase activity of E_{324S} is determined by Assay J described under item 4.5.14.6.

### 4.5.14.7.9 Preferred Variants of SEQ ID NO: 25

According to the invention, the polypeptide sequence of the hydantoin racemase E₃ may also be a variant of SEQ ID NO: 25.

The hydantoin racemase E₃, the polypeptide sequence of which is SEQ ID NO: 25, is denoted as “E_{325S}”. Hydantoin racemases E₃, the polypeptide sequence of which is selected from variants of SEQ ID NO: 25, are generally denoted as “E_{325V}”.

A variant of the polypeptide sequence of SEQ ID NO: 25 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 25.

The polypeptide sequence of a variant of the polypeptide sequence SEQ ID NO: 25 is not identical to SEQ ID NO: 25.

According to the invention, a hydantoin racemase E_{325V} has hydantoin racemase activity, determined as described under item 4.5.14.4 and 4.5.14.5.

According to the invention, a hydantoin racemase E_{325V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{325S}, wherein the hydantoin racemase activity of E_{325V}, relative to the hydantoin racemase activity E_{325S} is determined by Assay J described under item 4.5.14.6.

It is even more preferably according to the invention, that a hydantoin racemase E_{325V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{325S}, wherein the hydantoin racemase activity of E_{325V}, relative to the hydantoin racemase activity of E_{325S} is determined by Assay J described under item 4.5.14.6.

### 4.5.14.7.10 Preferred Variants of SEQ ID NO: 26

According to the invention, the polypeptide sequence of the hydantoin racemase E₃ may also be a variant of SEQ ID NO: 26.

The hydantoin racemase E₃, the polypeptide sequence of which is SEQ ID NO: 26, is denoted as “E_{326S}”. Hydantoin racemases E₃, the polypeptide sequence of which is selected from variants of SEQ ID NO: 26, are generally denoted as “E_{326V}”.

A variant of the polypeptide sequence of SEQ ID NO: 26 is a polypeptide with sequence identity of at least 60%, preferably ≥65%, more preferably ≥70%, more preferably ≥75%, more preferably ≥80%, more preferably ≥85%, more preferably ≥90%, more preferably ≥91%, more preferably ≥92%, more preferably ≥93%, more preferably ≥94%, more preferably ≥95%, more preferably ≥96%, more preferably ≥97%, more preferably ≥98%, more preferably ≥99%, more preferably ≥99.9% sequence identity to polypeptide sequence SEQ ID NO: 26.

The polypeptide sequence of a variant of the polypeptide sequence SEQ ID NO: 26 is not identical to SEQ ID NO: 26.

According to the invention, a hydantoin racemase E_{326V} has hydantoin racemase activity, determined as described under item 4.5.14.4 and 4.5.14.5.

According to the invention, a hydantoin racemase E_{326V} preferably has hydantoin racemase activity of at least 1%, preferably of at least 10%, more preferably of at least 20%, more preferably of at least 30%, more preferably of at least 40%, more preferably of at least 50%, more preferably of at least 60%, more preferably of at least 70%, more preferably of at least 80%, more preferably of at least 90%, more preferably of at least 99%, more preferably of at least 100% the hydantoin racemase of the hydantoin racemase E_{326S}, wherein the hydantoin racemase activity of E_{326V}, relative to the hydantoin racemase activity E_{326S} is determined by Assay J described under item 4.5.14.6.

It is even more preferably according to the invention, that a hydantoin racemase E_{326V} has hydantoin racemase activity in the range of 1 to 1000%, preferably in the range of 5 to 500%, more preferably in the range of 10 to 400%, more preferably in the range of 40 to 200%, more preferably in the range of 50 to 150%, more preferably in the range of 60 to 140%, more preferably in the range of 70 to 130%, more preferably in the range of 80 to 120%, more preferably in the range of 90 to 110%, more preferably 100% the hydantoin racemase activity of the hydantoin racemase E_{326S}, wherein the hydantoin racemase activity of E_{326V}, relative to the hydantoin racemase activity of E_{326S} is determined by Assay J described under item 4.5.14.6.

### 4.5.15 Preferred Method Conditions in Step (a)

In this preferred embodiment, in which step (a) is catalyzed by a hydantoin racemase E₃, the reaction in step (a) may be carried out under conditions known to the skilled person.

The reaction medium is preferably aqueous, more preferably an aqueous buffer.

Exemplary buffers commonly used in biotransformation reactions and advantageously used herein include Tris, phosphate, or any of Good's buffers, such as 2-(N-morpholino)ethanesulfonic acid

(“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”). In some embodiments, ammonium can act as a buffer. One or more organic solvents can also be added to the reaction.

The buffer preferably contains metal salts, more preferably metal salts such as halogenides of monovalent or bivalent metals (e.g. CoCl₂, MnCl₂).

The concentration of these metal salts in the reaction medium is preferably in the range from 1 μM to 1 M, more preferably 1 mM to 100 mM, even more preferably 1 to 10 mM.

Preferably, step (a) of the method according to the first aspect of the invention is carried out in a phosphate buffer.

The pH of the reaction medium in step (a) of the method according to the first aspect of the invention is preferably in the range of from 2 to 10, more preferably in the range of from 5 to 8, more preferably 7.2 to 7.5, most preferably 7.5.

Preferably, step (a) of the method according to the first aspect of the invention is carried out at a temperature in the range of from 20° C. to 70° C., more preferably in the range of from 30° C. to 55° C., most preferably 50° C.

In this preferred embodiment, in which step (a) is catalyzed by a hydantoin racemase E₃, the preferred reaction conditions in step (a) are the same as described for steps (b) and (c), confer items 4.5.7 and 4.5.13, respectively. It is even more preferred to carry out step (a) concomitantly with steps (b) and (c).

Preferably, the total concentration of all hydantoin racemases E₃ in the reaction solution in step (a) is in the range of from 1 μM to 10 mM, preferably 10 μM to 1 mM, more preferably 0.1 mM to 0.5 mM, most preferably 0.4 mM.

In alternative preferred embodiments, the total concentration of all hydantoin racemases E₃ in the reaction solution in step (a) is in the range of from 1 μg/l to 10 g/l, preferably 0.1 mg/l to 5 g/l, more preferably 1 mg/l to 1 g/l, more preferably 5 mg/l to 500 mg/l.

Preferably, step (a) is carried out in the same reaction medium in which steps (c) and (b) are carried out. The advantage is that this allows for a one-pot synthesis in which all the steps (a), (b), and (c) are carried out.

Preferably, the initial concentration of all the compounds according to formula D-(III) in the reaction medium in step (a) is in the range of from 1 μM to 1 M, preferably of from 10 μM to 0.5 M, more preferably of from 0.1 mM to 0.1 M, more preferably of from 1 mM to 10 mM, most preferably 1.25 mM.

If compounds according to formula L-(III) are present in the reaction medium in step (a), the initial concentration of all the compounds according to formula L-(III) in the reaction medium is preferably from 1 to a 100 times the concentration of all the compounds according to formula D-(III), more preferably 1 to 10 times the concentration, even more preferably 1 to 2 times even more preferably the same as the concentration of all the compounds according to formula D-(III).

“Initial concentration of all the compounds according to formula L-(III)/D-(III)” refers to the concentration of the respective compound L-(III) or D-(III), respectively, in the reaction medium when the respective compounds are employed in step (a).

### 4.6 Second Aspect: Method for Production of an L-Glufosinate P-Alkyl Ester

In a second aspect, the present invention relates to a further method for the production of an L-glufosinate P-alkyl ester according to formula L-(I):

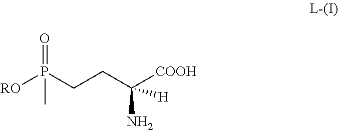

from a mixture M_{IIIA} comprising both enantiomers L-(III) and D-(III):

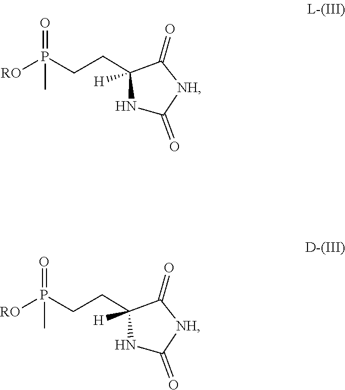

wherein R in formulae L-(I), L-(III), D-(III) is an alkyl group or aryl group. In particular, R is selected from the group consisting of alkyl group, phenyl group, benzyl group. Preferably, R is an alkyl group, more preferably an alkyl group with 1 to 10, even more preferably with 1 to 6, even more preferably with 1 to 4 carbon atoms. Even more preferably R═ethyl or n-butyl, most preferably R═n-butyl.

In this method according to the second aspect of the invention, a mixture M_{IIIA} comprising both enantiomers L-(III) and D-(III), wherein:

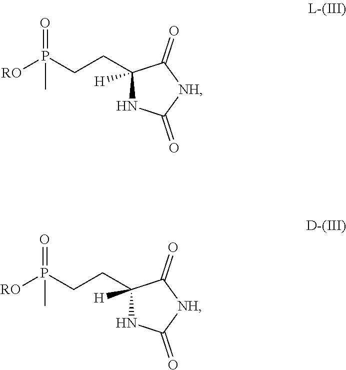

is provided, and subjected to at least two enzymatic reactions (ii) and (iii), giving a composition M_{I} comprising L-(I) and optionally D-(I). Therefore, the reaction according to the preferred second aspect of the invention is enzymatic.

The method for the production of an L-glufosinate P-alkyl ester according to formula L-(I), according to the second aspect of the invention is L-enantioselective, in particular L-enantiospecific, because M_{I} either comprises L-(I), but not D-(I) (in which case it is “L-enantiospecific”) or, when M_{I} comprises both enantiomers L-(I) and D-(I), the molar ratio of L-(I) to D-(I) in M_{I} is greater than the molar ratio of L-(III) to D-(III) in M_{IIIA} (in which case it is “L-enantioselective, but not L-enantiospecific”).

In a particular embodiment of the method according to the second aspect of the invention, M_{I} comprises both enantiomers L-(I) and D-(I) and the molar ratio of L-(I) to D-(I) in M_{I} is greater than the molar ratio of L-(III) to D-(III) in M_{IIIA} (in which case the method according to the second aspect of the invention is “L-enantioselective, but not L-enantiospecific”).

For sake of clarity it is pointed out that the feature “both enantiomers L-(III) and D-(III)” means that L-(III) is the enantiomer of D-(III), i.e. the only difference between L-(III) and D-(III) is the chirality at the sp³-hybridized carbon atom of the hydantoin (5-membered) ring.

Likewise, it is pointed out that the feature “both enantiomers L-(I) and D-(I)” means that L-(I) is the enantiomer of D-(I), i.e. the only difference between L-(I) and D-(I) is the chirality at the α carbon atom (i.e. the carbon atom adjacent to the carbonyl carbon atom).

In the method according to the second aspect of the invention, it is possible to enrich L-(I) over its enantiomer D-(I) in the product composition M_{I}, which means that the molar ratio of L-(I) to its enantiomer D-(I) in M_{I} obtained in step (iii) is greater than the molar ratio of L-(III) to its enantiomer D-(III) in mixture M_{IIIA} provided in step (i-A).

In particular, in case the method according to the second aspect of the invention is “L-enantioselective, but not L-enantiospecific”, the portion of L-enantiomers L-(I) per molecule D-enantiomer D-(I) in M_{I} is greater than the portion of L-enantiomers L-(III) per molecule D-enantiomer D-(III) in M_{IIIA}, in particular by a factor of X_{L}, wherein X_{L} is at least 1.1, preferably at least 1.5, more preferably at least 2, more preferably at least 5, even more preferably at least 9, even more preferably at least 99. In another preferred embodiment X_{L} is in the range of 1.1 to 99, preferably in the range of 1.5 to 9, even more preferably in the range of 2 to 5.

4.6.1 Step (i-A)

In a first step of the method according to the second aspect of the invention, a mixture M_{IIIA} comprising both enantiomers L-(III) and D-(III) is provided.

In a preferred embodiment, the mixture M_{IIIA} is a racemic mixture of enantiomer L-(III) and enantiomer D-(III), meaning that the molar ratio of enantiomer L-(III) to enantiomer D-(III) is essentially 1:1.

Alternatively, enantiomer D-(III) is comprised in an excess to L-(III) in mixture M_{IIIA}, meaning that, while L-(III) is present in mixture M_{IIIA}, the molar ratio of enantiomer L-(III) to enantiomer D-(III) in mixture M_{IIIA} is <1:1, preferably <0.9:1, more preferably <0.75:1, more preferably <0.5:1, more preferably <0.2:1, more preferably <0.1:1, more preferably <0.01:1.

In other preferred embodiments, the molar ratio of enantiomer L-(III) to enantiomer D-(III) in mixture M_{IIIA} is in the range of 3:2 to 1:99, more preferably in the range 1:1 to 1:99, more preferably in the range of 1.01:1 to 1:99, more preferably in the range 1:1.01 to 1:9, more preferably in the range 1:1.01 to 1:8, more preferably in the range 1:1.01 to 1:3.

A mixture of these enantiomers can be obtained by the skilled person, for example by organic synthesis as set forth in DE 31 42 036 A1 and by Slomka et al. E. Ware, Chem. Rev. 1950, 46, 403-470 and C. Avendaño & J. C. Menendez, “Hydantoin and its derivates” in Kirk-Othmer Encyclopedia of Chemical Technology 2000 give a general overview over hydantoin chemistry.

Preferably, the mixture M_{IIIA} is provided in step (i-A) in a reaction medium.

Namely, the reaction medium in step (i-A) is preferably aqueous, more preferably an aqueous buffer.

Exemplary buffers commonly used in biotransformation reactions and advantageously used in step (i-A) include Tris, phosphate, or any of Good's buffers, such as 2-(N-morpholino)ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”). In some embodiments, ammonium can act as a buffer. One or more organic solvents can also be added to the reaction.

The buffer preferably contains metal salts, more preferably metal salts such as halogenides of monovalent or bivalent metals (e.g. CoCl₂, MnCl₂).

The concentration of these metal salts in the reaction medium in step (i-A) is preferably in the range from 1 μM to 1 M, more preferably 1 mM to 100 mM, even more preferably 1 to 10 mM.

Preferably, step (i-A) of the method according to the second aspect of the invention is carried out in a phosphate buffer.

The pH of the reaction medium in step (i-A) of the method according to the first aspect of the invention is preferably in the range of from 2 to 10, more preferably in the range of from 5 to 8, more preferably 7.2 to 7.5, most preferably 7.5.

More preferably, the concentration of all the compounds according to formula D-(III) in the reaction medium provided in step (i-A) is in the range of from 1 μM to 1 M, preferably of from 10 μM to 0.5 M, more preferably of from 0.1 mM to 0.1 M, more preferably of from 1 mM to 10 mM, most preferably 1.25 mM.

Concerning the compounds according to formula L-(III) that are present in the reaction medium preferably provided in step (i-A), the concentration of all the compounds according to formula L-(III) in the reaction medium is preferably from 1 to a 100 times the concentration of all the compounds according to formula D-(III), more preferably 1 to 10 times the concentration, even more preferably 1 to 2 times even more preferably the same as the concentration of all the compounds according to formula D-(III).

4.6.2 Optional Step (i-B)

In an optional step (i-B), at least a part of the compounds D-(III) comprised by the mixture M_{IIIA} are reacted into L-(III), giving a composition M_{IIIB} comprising L-(III) and optionally its enantiomer D-(III). It goes without saying that the molar ratio of enantiomer L-(III) to enantiomer D-(III) in mixture M_{IIIB} is greater than in mixture M_{IIIA}.

In case step (i-B) is carried out, it is preferably carried out as described for step (a) under items 4.5.14 and 4.5.15.

In particular, the reaction according to step (i-B) is catalyzed by a hydantoin racemase E₃ as described with respect to step (a).

Preferably, the hydantoin racemase E₃ used in step (i-B) is categorized in the EC class 5.1.99.5. In another embodiment, the polypeptide sequence of the hydantoin racemase E₃ used in step (i-B) is selected from the group consisting of SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, SEQ ID NO: 26 and variants thereof, most preferably selected from SEQ ID NO: 17 and variants thereof.

Preferably, the hydantoin racemase E₃ used in step (i-B) is selected from the group consisting of E_{317S} and E_{317V} (both described under item 4.5.14.7.1), E_{318S} and E_{318V} (both described under item 4.5.14.7.2), E_{319S} and E_{319V} (both described under item 4.5.14.7.3), E_{320S} and E_{320V} (both described under item 4.5.14.7.4), E_{321S} and E_{321V} (both described under item 4.5.14.7.5), E_{322S} and E_{322V} (both described under item 4.5.14.7.6), E_{323S} and E_{323V} (both described under item 4.5.14.7.7), E_{324S} and E_{324V} (both described under item 4.5.14.7.8), E_{325S} and E_{325V} (both described under item 4.5.14.7.9), E_{326S} and E_{326V} (both described under item 4.5.14.7.10), more preferably from E_{317S} and E_{317V}, even more preferably from E_{317S}.

The reaction conditions in step (i-B) are preferably as described with respect to step (a) under item 4.5.15.

Namely, the reaction medium in step (i-B) is preferably aqueous, more preferably an aqueous buffer.

Exemplary buffers commonly used in biotransformation reactions and advantageously used in step (i-B) include Tris, phosphate, or any of Good's buffers, such as 2-(N-morpholino)ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”).

In some embodiments, ammonium can act as a buffer. One or more organic solvents can also be added to the reaction.

The buffer preferably contains metal salts, more preferably metal salts such as halogenides of monovalent or bivalent metals (e.g. CoCl₂, MnCl₂).

The concentration of these metal salts in the reaction medium in step (i-B) is preferably in the range from 1 μM to 1 M, more preferably 1 mM to 100 mM, even more preferably 1 to 10 mM.

Preferably, step (i-B) of the method according to the second aspect of the invention is carried out in a phosphate buffer.

The pH of the reaction medium in step (i-B) of the method according to the first aspect of the invention is preferably in the range of from 2 to 10, more preferably in the range of from 5 to 8, more preferably 7.2 to 7.5, most preferably 7.5.

Preferably, step (i-B) of the method according to the first aspect of the invention is carried out at a temperature in the range of from 20° C. to 70° C., more preferably in the range of from 30° C. to 55° C., most preferably 50° C.

Preferably, the total concentration of all hydantoin racemases E₃ in the reaction solution in step (i-B) is in the range of from 1 μM to 10 mM, preferably 10 μM to 1 mM, more preferably 0.1 mM to 0.5 mM, most preferably 0.4 mM.

In alternative preferred embodiments, the total concentration of all hydantoin racemases E₃ in the reaction solution in step (i-B) is in the range of from 1 μg/l to 10 g/l, preferably 0.1 mg/l to 5 g/l, more preferably 1 mg/l to 1 g/l, more preferably 5 mg/l to 500 mg/l.

Preferably, step (i-B) is carried out in the same reaction medium in which steps (ii) and (iii) are carried out. The advantage is that this allows for a one-pot synthesis in which all the steps (i-B), (ii), and (iii) are carried out.

At the end of optional step (i-B), a composition M_{IIIB} comprising L-(III) and optionally its enantiomer D-(III) is obtained, preferably a mixture M_{IIIB} comprising L-(III) and its enantiomer D-(III) is obtained.

### 4.6.3 Step (ii)

In step (ii) of the method according to the second aspect of the invention, the mixture M_{IIIa} or, in case step (i-B) is carried out, composition M_{IIIB}, is subjected to step (b) as described above under item 4.5.8-4.5.13.

In other words: in those embodiments, in which step (i-B) is not carried out, M_{IIIA} is subjected to step (b) as described above under item 4.5.8-4.5.13.

In case step (i-B) is carried out, Mus obtained after step (i-B) is subjected to step (b) as described above under item 4.5.8-4.5.13.

The reaction according to step (ii) is catalyzed by a hydantoinase E₂ as described with respect to step (b), preferably by an L-hydantoinase E₂ as described with respect to step (b).

More preferably, the hydantoinase E₂, even more preferably the L-hydantoinase E₂ used in step (ii) is categorized in the EC class 3.5.2.2.

In another preferred embodiment, the polypeptide sequence of the hydantoinase E₂, even more preferably the L-hydantoinase E₂ used in step (ii) is selected from the group consisting of SEQ ID NO: 10 and variants thereof, SEQ ID NO: 11 and variants thereof, SEQ ID NO: 12 and variants thereof, SEQ ID NO: 13 and variants thereof, SEQ ID NO: 14 and variants thereof, SEQ ID NO: 15 and variants thereof, SEQ ID NO: 16 and variants thereof, even more preferably from SEQ ID NO: 10 and variants thereof.

Preferably, the hydantoinase E₂, even more preferably the L-hydantoinase E₂ used in step (ii) is selected from the group consisting of E_{210S} and E_{210V} (both described under item 4.5.12.1), E₂₁₁S and E_{211V} (both described under item 4.5.12.2), E_{212S} and E_{212V} (both described under item 4.5.12.3), E_{213S} and E_{213V} (both described under item 4.5.12.4), E_{214S} and E_{214V} (both described under item 4.5.12.5), E_{215S} and E_{215V} (both described under item 4.5.12.6), E_{216S} and E_{216V} (both described under item 4.5.12.7), more preferably from E_{210S} and E_{210V}, even more preferably from E_{210S}.

The reaction conditions in step (ii) are preferably as described with respect to step (b) under item 4.5.13.

Namely, the reaction medium in step (ii) is preferably aqueous, more preferably an aqueous buffer.

Exemplary buffers commonly used in biotransformation reactions and advantageously used in step (ii) include Tris, phosphate, or any of Good's buffers, such as 2-(N-morpholino)ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”). In some embodiments, ammonium can act as a buffer. One or more organic solvents can also be added to the reaction.

The buffer preferably contains metal salts, more preferably metal salts such as halogenides of monovalent or bivalent metals (e.g. CoCl₂, MnCl₂).

The concentration of these metal salts in the reaction medium is preferably in the range from 1 μM to 1 M, more preferably 1 mM to 100 mM, even more preferably 1 to 10 mM.

Preferably, step (ii) of the method according to the second aspect of the invention is carried out in a phosphate buffer.

The pH of the reaction medium in step (ii) of the method according to the first aspect of the invention is preferably in the range of from 2 to 10, more preferably in the range of from 5 to 8, more preferably 7.2 to 7.5, most preferably 7.5.

Preferably, step (ii) of the method according to the first aspect of the invention is carried out at a temperature in the range of from 20° C. to 70° C., more preferably in the range of from 30° C. to 55° C., most preferably 50° C.

Preferably, the total concentration of all hydantoinases E₂ in the reaction solution in step (ii) is in the range of from 1 μM to 10 mM, preferably 10 μM to 1 mM, more preferably 0.1 mM to 0.5 mM, most preferably 0.4 mM.

In alternative preferred embodiments, the total concentration of all hydantoinases E₂ in the reaction solution in step (ii) is in the range of from 1 μg/l to 10 g/l, preferably 0.1 mg/l to 5 g/l, more preferably 1 mg/l to 1 g/l, more preferably 5 mg/l to 500 mg/l.

Preferably, step (ii) is carried out in the same reaction medium in which step (iii) is carried out.

At the end of step (ii), a composition M_{II} comprising L-(II) and optionally its enantiomer D-(II) is obtained, preferably a mixture M_{I} comprising L-(II) and its enantiomer D-(II) is obtained:

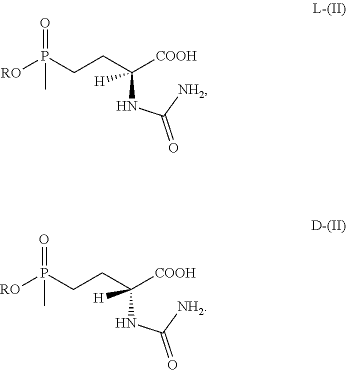

R in formulae L-(II) and D-(II) in composition M_{II} has the same meaning as described for L-(I).

4.6.4 Step (iii)

In step (iii) of the method according to the second aspect of the invention, M_{II} obtained in step (ii) is subjected to step (c) as described above under items 4.5.1 to 4.5.7 wherein the reaction according to step (iii) is catalyzed by an L-carbamoylase E₁.

This brings about that the L-enantiomer L-(I) is enriched over the D-enantiomer D-(I) in M_{I}, i.e., in case M_{I} comprises both enantiomers L-(I) and D-(I), the molar ratio of L-(I) to D-(I) in M_{I} is greater than the molar ratio of L-(III) to D-(III) in M_{IIIA}.

More preferably, the L-carbamoylase E₁ used in step (iii) is categorized in the EC class 3.5.1.87. In another preferred embodiment, the polypeptide sequence of the L-carbamoylase E₁ used in step (iii) is selected from the group consisting of SEQ ID NO: 1 and variants thereof, SEQ ID NO: 2 and variants thereof, SEQ ID NO: 3 and variants thereof, SEQ ID NO: 4 and variants thereof, SEQ ID NO: 5 and variants thereof, SEQ ID NO: 6 and variants thereof, SEQ ID NO: 7 and variants thereof, SEQ ID NO: 8 and variants thereof, SEQ ID NO: 9 and variants thereof.

Preferably, the L-carbamoylase E₁ used in step (iii) is selected from the group consisting of E₁₀₁S and E_{101V} (both described under item 4.5.6.1), E_{102S} and E_{102V} (both described under item 4.5.6.2), E_{103S} and E_{103V} (both described under item 4.5.6.3), E_{104S} and E_{104V} (both described under item 4.5.6.4), E_{105S} and E_{105V} (both described under item 4.5.6.5), E_{106S} and E_{106V} (both described under item 4.5.6.6), E_{107S} and E_{107V} (both described under item 4.5.6.7), E_{108S} and E_{108V} (both described under item 4.5.6.8), E_{109S} and E_{109V} (both described under item 4.5.6.9), more preferably from E₁₀₁S and E_{101V}, even more preferably from E₁₀₁S.

The reaction conditions in step (iii) are preferably as described with respect to step (c) under item 4.5.7.

Namely, the reaction medium in step (iii) is preferably aqueous, more preferably an aqueous buffer.

Exemplary buffers commonly used in biotransformation reactions and advantageously used in step (iii) include Tris, phosphate, or any of Good's buffers, such as 2-(N-morpholino)ethanesulfonic acid (“MES”), N-(2-acetamido)iminodiacetic acid (“ADA”), piperazine-N,N-bis(2-ethanesulfonic acid) (“PIPES”), N-(2-acetamido)-2-aminoethanesulfonic acid (“ACES”), P-hydroxy-4-morpholinepropanesulfonic acid (“MOPSO”), cholamine chloride, 3-(N-morpholino)propanesulfonic acid (“MOPS”), N,N-Bis(2-hydroxyethyl)-2-aminoethanesulfonic acid (“BES”), 2-[1,3-dihydroxy-2-(hydroxymethyl)propan-2-yl]amino]ethanesulfonic acid (“TES”), 4-(2-hydroxyethyl)-1-piperazineethanesulfonic acid (“HEPES”), 3-(Bis(2-hydroxyethyl)amino)-2-hydroxypropane-1-sulfonic acid (“DIPSO”), acetamidoglycine, 3-(N-Tris(hydroxymethyl)methylamino(-2-hydroxypropane)sulfonic acid (“TAPSO”), piperazine-N,N-bis(2-hydroxypropanesulfonic acid) (“POPSO”), 4-(2-Hydroxyethyl)piperazine-1-(2-hydroxypropanesulfonic acid) (“HEPPSO”), 3-[4-(2-Hydroxyethyl)-1-piperazinyl]propanesulfonic acid (“HEPPS”), tricine, glycinamide, bicine, or 3-[1,3-dihydroxy-2-(hydroxymethyl)propan-2-yl]amino]propane-1-sulfonic acid (“TAPS”).

In some embodiments, ammonium can act as a buffer. One or more organic solvents can also be added to the reaction.

The buffer preferably contains metal salts, more preferably metal salts such as halogenides of monovalent or bivalent metals (e.g. CoCl₂, MnCl₂).

The concentration of these metal salts in the reaction medium is preferably in the range from 1 μM to 1 M, more preferably 1 mM to 100 mM, even more preferably 1 to 10 mM.

Preferably, step (iii) of the method according to the second aspect of the invention is carried out in a phosphate buffer.

The pH of the reaction medium in step (iii) of the method according to the first aspect of the invention is preferably in the range of from 2 to 10, more preferably in the range of from 5 to 8, more preferably 7.2 to 7.5, most preferably 7.5.

Preferably, step (iii) of the method according to the first aspect of the invention is carried out at a temperature in the range of from 20° C. to 70° C., more preferably in the range of from 30° C. to 55° C., most preferably 50° C.

Preferably, the total concentration of all carbamoylases E₁ in the reaction solution in step (iii) is in the range of from 1 μM to 10 mM, preferably 10 μM to 1 mM, more preferably 0.1 mM to 0.5 mM, most preferably 0.4 mM.

In alternative preferred embodiments, the total concentration of all carbamoylases E₁ in the reaction solution in step (iii) is in the range of from 1 μg/l to 10 g/l, preferably 0.1 mg/l to 5 g/l, more preferably 1 mg/l to 1 g/l, more preferably 5 mg/l to 500 mg/l.

At the end of step (i), a composition M_{I} comprising L-(I) and optionally its enantiomer D-(I) is obtained, preferably a mixture M_{I} comprising L-(I) and its enantiomer D-(I) is obtained.

### 4.7 Saponification

The L-glufosinate P-alkyl ester according to formula L-(I):produced with the method according to the first or second aspect can then be saponified to produce LGA, for example in an acidic aqueous medium, preferably at pH<7, even more preferably at an pH between <6, more preferably at an pH<3, even more preferably at an pH of <1. These saponification conditions are known to the skilled person and described e.g. by H. J. Zeiss, J. Org. Chem. 1991, 56, 1783-1788.

### 5. Examples

### 5.1 Example 1 Identification of Suitable Enzymes and Construction of Plasmids

Genes of different origins encoding a hydantoinase (dihydropyrimidinase, EC 3.5.2.2), L-carbamoylase (N-carbamoyl-L-amino-acid hydrolase, EC 3.5.1.87) and hydantoin racemase (EC 5.1.99.5) were tested for their ability to react with different hydantoin substrates according to structure L-(III) and D-(III) to form the respective enantioselective L-glufosinate derivative according to structure L-(I).

### 5.1.1 Examined Enzymes

Details of the strains and genes of the respective enzymes that were used in the examples are summarized in table 4.

TABLE 4

| | | | | SEQ ID NO: | SEQ ID NO: |
|---|---|---|---|---|---|
| | | | | of the poly- | of the poly- |
| Plasmid | Enzyme | Donor Strain | Gene name | nucleotides | peptides |
| pOM22c | L-carbamoylase | Arthrobacter | hyuC | SEQ ID NO: 27 | SEQ ID NO: 1 |
| | | sp. DSM 3747 | | | |
| pOM22c | hydantoinase | Arthrobacter | hyuH | SEQ ID NO: 28 | SEQ ID NO: 10 |
| | | sp. DSM 9771 | | | |
| pOM21c | hydantoin racemase | Arthrobacter | hyuR | SEQ ID NO: 29 | SEQ ID NO: 17 |
| | | sp. DSM 3745 | | | |

### 5.1.2 Cloning of the Enzymes

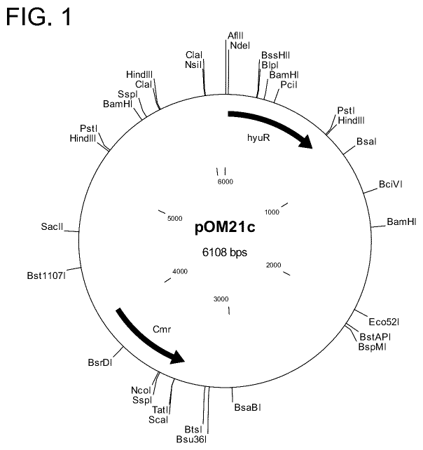

Cloning of the hydantoin racemase and generation of the plasmid pOM21c (FIG. 1) was carried out as described in example 1 of WO 2004/111227 A2. In particular, a polynucleotide was used which comprised the respective gene (SEQ ID NO: 29) and additional sequences for Ndel and Pstl restriction sites.

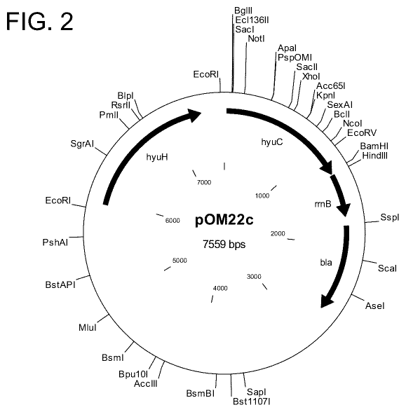

Cloning of hydantoinase and L-carbamoylase into the rhamnose expression vector pJOE4036 was carried out in a plasmid derivative of the rhamnose expression vector pJOE4036. Polynucleotides comprising the genes of the respective enzymes (SEQ ID NOs: 27, 28) were synthesized by GeneArt (ThermoFisher Scientific (Waltham, USA)). The polynucleotides carried additional sequences for EcoR1 and HindIII restriction sites. Both enzymes were cloned into pJOE4036 using those restriction sites resulting in the plasmid pOM22c, under the control of a rhamnose promotor (FIG. 2).

### 5.2 Example 2: Production of Strains Positive for Hydantoinase, L-Carbamoylase and Hydantoin Racemase

Chemically competent E. coli ET5 cells (as described in WO 2004/042047 A1) were transformed with 10 ng of the plasmid pOM22c generated according to Example 1.

The generated strain which was positive for hydantoinase- and carbamoylase was rendered chemically competent and transformed with 10 ng of the plasmid pOM21c.

An E. coli ET5 strain transformed with pOM21c or pOM22c was incubated under shaking (250 U/min) at 30° C. for 18 hours in LB medium containing ampicillin (100 μg/l), chloramphenicol (50 μg/l), and 2 g/l rhamnose.

The biomass was separated by centrifugation, resuspended in 50 mM phosphate buffer (pH 7.2) and applied in biotransformation tests in the following examples. The concentration of the biomass in the solution was 40-50 g/l. The solution was used as catalyst (“catalyst 1”) in the following.

The concentration of the respective polypeptide carbamoylase, hydantoinase and racemase in the obtained solution may be determined by SDS page and analysis of the respective bands via the software GelQuant® (BiochemLabSolutions).

### 5.3 Inventive Examples 11 and 12 and Comparative Examples C1 and C2: Production of P-Alkyl Phosphinothricin Ester Using the Strains Produced in Examples 1 and 2

In the following examples, different hydantoin substrates were tested to determine whether the respective polpypetide catalyzed the reaction of the respective substrate enantioselectively to give the corresponding L amino acid.

### 5.3.1 Comparative Example C1

In comparative example C1, a racemic mixture M_{C1} of 1.25 mmol L-(IV)_{c1} and 1.25 mmol D-(IV)_{c1}, wherein L-(IV)_{c1} and D-(IV)_{c1} are hydantoins with the following formulae, was used as substrate:

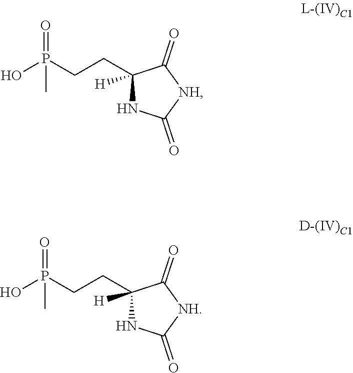

The mixture M_{C1} was dissolved in a stirring reactor with 25 ml water. 2.4 g of the catalyst 1 was added, followed by the addition of 50 μl CoCl₂ Solution. The suspension was set to pH 7.5 with 0.5 M NaOH. The total volume was replenished with water to 50 ml, so that the concentration of each enantiomer L-(IV)_{c1} and D-(IV)_{c1} was 0.025 mol/l and the concentration of CoCl₂ in the final solution was 1 mM. v. The pH was held between 7.0 and 7.5 by HCl-titration or NaOH-titration. The temperature was maintained at 37° C. by a thermostat during the reaction.

The reaction was stopped after 120 hours by addition of 2 N HCl until pH 2.5 was reached. The biomass was separated by centrifugation or filtration.

The enzyme reaction was monitored by ninhydrin test to determine the formation of amino acids. No formation of amino acids was detected by the ninhydrine test.

### 5.3.2 Comparative Example C2

In comparative example C2, a racemic mixture M_{C2} of 1.25 mmol L-(V)_{C2} and 1.25 mmol D-(V)_{C2}, wherein L-(V)_{C2} and D-(V)_{C2} are carbamoylates with the following formulae, was used as substrate:

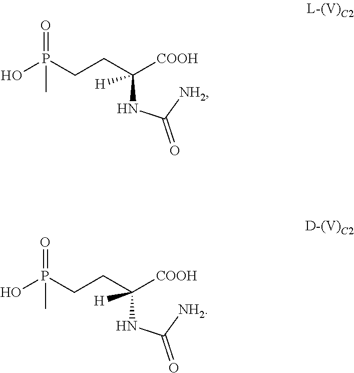

The mixture M_{C2} was dissolved in a stirring reactor with 25 ml water. 2.4 g of the catalyst 1 was added, followed by the addition of 50 μl CoCl₂ Solution. The suspension was set to pH 7.5 with 0.5 M NaOH. The total volume was replenished with water to 50 ml, so that the concentration of each enantiomer L-(V)_{C2} and D-(V)_{C2} was 0.025 mol/l and the concentration of CoCl₂ in the final solution was 1 mM. The pH was held between 7.0 and 7.5 by HCl-titration or NaOH-titration. The temperature was maintained at 37° C. by a thermostat during the reaction.

The reaction was stopped after 120 hours by addition of 2 N HCl until pH 2.5 was reached. The biomass was separated by centrifugation or filtration.

The enzyme reaction was monitored by ninhydrin test to determine the formation of amino acids. No formation of amino acids was detected by the ninhydrine test.

### 5.3.3 Inventive Example 11

In inventive example 11, a racemic mixture M_{I1} of 1.25 mmol L-(VI)_{I1} and 1.25 mmol D-(VI)_{I1}, wherein L-(VI)_{I1} and D-(VI)_{I1} were hydantoins with the following formulae, was used as substrate:

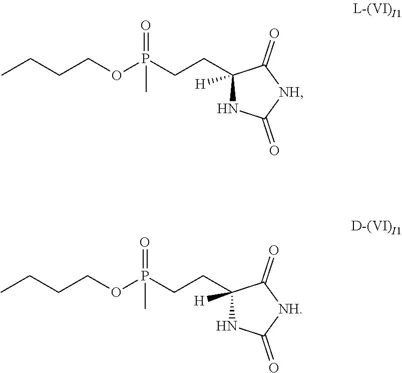 [SMILES: CCCCOP(C)(=O)CC[C@@H]1NC(=O)NC1=O ; CCCCOP(C)(=O)CC[C@H]1NC(=O)NC1=O]

The mixture M_{I1} was dissolved in a stirring reactor with 25 ml water. 2.4 g of the catalyst 1 was added, followed by the addition of 50 μl CoCl₂ Solution. The suspension was set to pH 7.5 with 0.5 M NaOH. The total volume was replenished with water to 50 ml, so that the concentration of each enantiomer L-(VI)_{I1} and D-(VI)_{I1} was 0.025 mol/l and the concentration of CoCl₂ in the final solution was 1 mM. The pH was held between 7.0 and 7.5 by HCl-titration or NaOH-titration. The temperature was maintained at 37° C. by a thermostat during the reaction.

The reaction was stopped after 120 hours by addition of 2 N HCl until pH 2.5 was reached. The biomass was separated by centrifugation or filtration.

The enzyme reaction was monitored by ninhydrin test to determine the formation of amino acids. Formation of amino acids was detected by the ninhydrine test.

In a final step the reaction mixture was saponified at 100° C. for 10 hours by adding 6 M HCl to obtain L-glufosinate and, if present, D-glufosinate. The final reaction mixture was analysed by LC-MS with a CR-I column as described under item 5.4 to determine the enantiomeric excess (“ee”) of either D- or L-glufosinate. An ee of LGA of 79% over the D-enantiomer was detected. The ee of the L-enantiomer (“ee_{L}”) is determined by the following formula in the context of the invention, wherein m_{L} and m_{D} are the detected molar masses of L- and D-glufosinate, respectively:

$$e {e}_{L} = \frac{( {m}_{L} - {m}_{D} ) \times 100 \%}{( {m}_{L} + {m}_{D} )} .$$

### 5.3.4 Inventive Example 12

In inventive example I2, a racemic mixture M_{I2} of 1.25 mmol L-(VII)_{I2} and 1.25 mmol D-(VII)_{I2}, wherein L-(VII)_{I2} and D-(VII)_{I2} were hydantoins with the following formulae, was used as substrate:

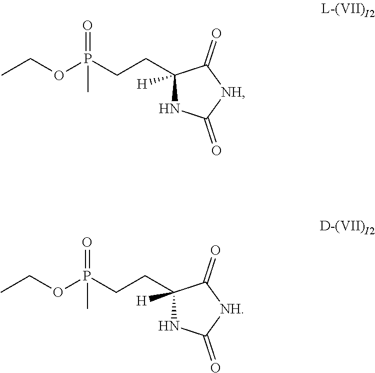

The mixture M_{I2} was dissolved in a stirring reactor with 25 ml water. 2.4 g of the catalyst 1 was added, followed by the addition of 50 μl CoCl₂ Solution. The suspension was set to pH 7.5 with 0.5 M NaOH. The total volume was replenished with water to 50 ml, so that the concentration of each enantiomer L-(VII)₁₂ and D-(VII) was 0.025 mol/l and the concentration of CoCl₂ in the final solution was 1 mM. The pH was held between 7.0 and 7.5 by HCl-titration or NaOH-titration. The temperature was maintained at 37° C. by a thermostat during the reaction.

The reaction was stopped after 120 hours by addition of 2 N HCl until pH 2.5 was reached. The biomass was separated by centrifugation or filtration.

The enzyme reaction was monitored by ninhydrin test to determine the formation of amino acids. Formation of amino acids was detected by the ninhydrine test.

In a final step the reaction mixture was saponified at 100° C. for 10 hours by adding 6 M HCl to obtain L-glufosinate and, if present, D-glufosinate. The final reaction mixture was analysed by LC-MS with a CR-I column as described under item 5.4 to determine the enantiomeric excess (“ee”) of either D- or L-glufosinate. An ee of LGA of 77% over the D-enantiomer was detected.

### 5.3.5 Results

The results of the examples C1, C2, 11, 12 are summarized in table 5.

TABLE 5

| Example | substrate | Ninhydrine test | ee_{L} |
|---|---|---|---|
| C1 | L-(IV)_{C1}, D-(IV)_{C1} | Negative | — |
| C2 | L-(V)_{C2}, D-(V)_{C2} | Negative | — |
| I1 | L-(VI)_{I1}, D-(VI)_{I1} | Positive | 79% |
| I2 | L-(VII)_{I2}, D-(VII)_{I2} | Positive | 77% |

### 5.3.6 Conclusion 1

The results as summarized in table 5 show that certain hydantoin esters such as the alkylated hydantoin esters can be used as substrates for the enzymatic enantioselective synthesis of L-glufosinate (11, 12). In contrast, substrates in which the phosphinic acid group is not protected are not accepted by the catalytic system (C1, C2). In this regard, C2 suggests that at least one reason for this is that the L-carbamoylase enzyme does not accept the respective substrate in which the phosphinic acid function is not protected by an ester group.

Moreover, in contrast to the process for enantioselective production according to the prior art (CN 111662325 A), the ee excess could be maintained throughout the process, because saponification is carried out not at the hydantoin stage, but at the stage of the amino acid ester.

### 5.4 Analytical Methods

### Analysis Method 1

L-glufosinate and D-glufosinate were detected by LC-MS (“Liquid Chromatography —Mass Spectrometry”) with a chiral column [Daicel CROWNPAK CR-I-(−)] as follows. For hydantoins, a Daicel Chiralpak IA-U column may also be used.

This detection method may also be used for detection and quantification of the
the LGA P-(n-butyl) ester according to formula L-(I), wherein R═n-butyl, according to Assay A_{L} (item 4.5.3.1);
the D-glufosinate P-(n-butyl) ester according to formula D-(I), wherein R═n-butyl, according to Assay A_{D} (item 4.5.3.2);
the carbamoyl LGA P-(n-butyl) ester according to formula L-(II), wherein R═n-butyl, according to Assay D_{L} (item 4.5.9.1);
the carbamoyl D-glufosinate P-(n-butyl) ester according to formula D-(II), wherein R═n-butyl, according to Assay D_{D} (item 4.5.9.2);
the L-enantiomer of a n-butyl ester of hydantoin glufosinate of the formula L-(III), wherein R═n-butyl, according to Assay G (item 4.5.14.3).
the D-enantiomer of a n-butyl ester of hydantoin glufosinate of the formula D-(III), wherein R═n-butyl, according to Assay G (item 4.5.14.3).

### 5.4.1 Acquisition Method Details

CR-I-(−), 3.0 mm I.D. ×150 mm, 5 μm, Crownpak, Part. No. 54784
MS QQQ Mass Spectrometer 6420, Agilent
ESI+
MS2 SIM, Glufosinat m/z 182.06 Unit Positive
Centroid

### 5.4.2 Source Parameter

Parameter Value (+); Gas Temp (° C.): 350; Gas Flow (l/min): 12; Nebulizer (psi): 25; Capillary (V): 3000; Sampler Module: G1329B; Injection Volume 1.00 μL; 5° C.

### 5.4.3 Binary Pump Module

G1312B
Flow: 0.200 mL/min;
Channel A: H₂O with trifluoroacetic acid (TFA) pH1.15;
Channel B: ACN (acetonitrile);
isocratic 80.0% A/20.0% B; stoptime 7.00 min
Column Comp. Module: G1316A
Temperature 5.0° C.

### Analysis Method 2

A further analytical method was used to determine the yield of the LGA P-(n-butyl) ester according to formula L-(I), wherein R═n-butyl, and the D-glufosinate P-(n-butyl) ester according to formula D-(I), wherein R═n-butyl. In particular, this method was used to determine the yield of L-(VIII)₁₃ and D-(VIII)₁₃, as summarized in table 6 hereinafter.

In this method, LC-MS (“Liquid Chromatography —Mass Spectrometry”) with a chiral column [Chiralpak 3 μm ZWIX (+), 150×3 mm, DAICEL] was used as follows.

### 5.4.4 Acquisition Method Details

ZWIX (+), 150×3 mm; 3 μm
MS QQQ Mass Spectrometer 6420, Agilent
ESI+
MS2 SIM, Glufosinat-carbamoyl-butyl ester m/z 238.1 Unit Positive
Centroid

### 5.4.5 Source Parameter

Parameter Value (+); Gas Temp (° C.): 350; Gas Flow (I/min): 12; Nebulizer (psi): 50; Capillary (V): 4000; Sampler Module: G1329B; Injection Volume 1.00 μL; 5° C.

### 5.4.6 Binary Pump Module

G1312B
Flow: 0.300 mL/min;
isocratic 49.0% acetonitrile/49.0% methanol, 2.0% H₂O; stoptime 15.00 min
Column Comp. Module: G1316A
Temperature 5.0° C.

### 5.5 Example 3 Identification of Further Carbamoylase Enzymes and Construction of Plasmids

In a further test series, genes encoding L-carbamoylases (N-carbamoyl-L-amino-acid hydrolases, EC 3.5.1.87) of different origins were tested for their ability to react with different carbamoyl substrates according to structure L-(II) and D-(II) to form the respective L-glufosinate/D-glufosinate derivative according to structures L-(I) and D-(I), respectively. Cloning and expression of the respective L-carbamoylase gene was essentially carried out as described by B. Wilms, A. Wiese, C. Syldatk, R. Mattes, J. Altenbuchner, M. Pietzsch, Journal of Biotechnology 1999, 68, 101-113 (hereinafter “Wilms et al.”), in particular, as set forth in the following.

### 5.5.1 Examined Enzymes

Details of the strains and genes of the respective L-carbamoylase that were used in the examples are summarized in table 5.

TABLE 5

| | | | SEQ ID NO: |
|---|---|---|---|
| | | Gene | of the |
| Plasmid | Donor Strain | name | polypeptides |
| pOM17c | Arthrobacter | hyuC | SEQ ID NO: 1 |
| | sp. DSM 3747 | | |
| pOM17c | Geobacillus | amaB | SEQ ID NO: 2 |
| {Prha}[amaB_Gst] | stearothermophilus | | |
| pOM17c | Pseudomonas | atcC | SEQ ID NO: 3 |
| {Prha}[atc_Ps] | sp. QR-101 | | |
| pOM17c | Paenarthrobacter | hyuC | SEQ ID NO: 5 |
| {Prha}[hyuC_Pau] | aurescens | | |
| pOM17c | Arthrobacter | hyuC | SEQ ID NO: 8 |
| {Prha}[hyuC_Asp | sp. BT801 | | |
| (co_Ec)] | | | |

### 5.5.2 Cloning of the Enzymes

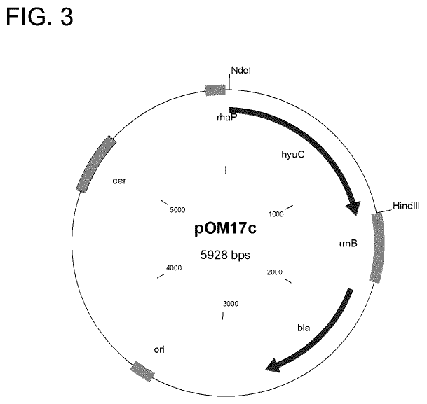

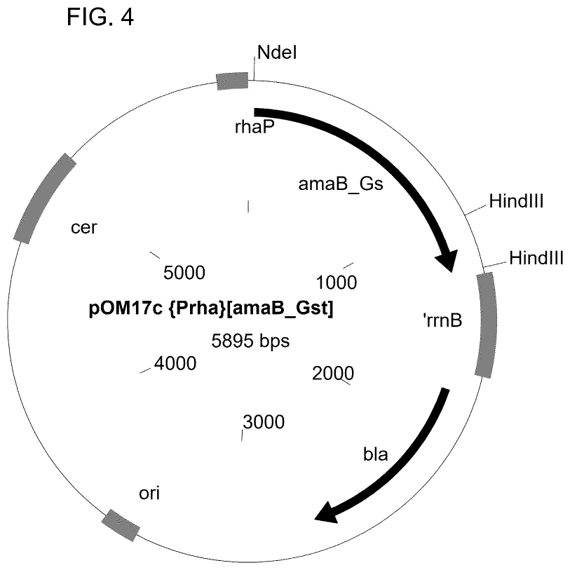

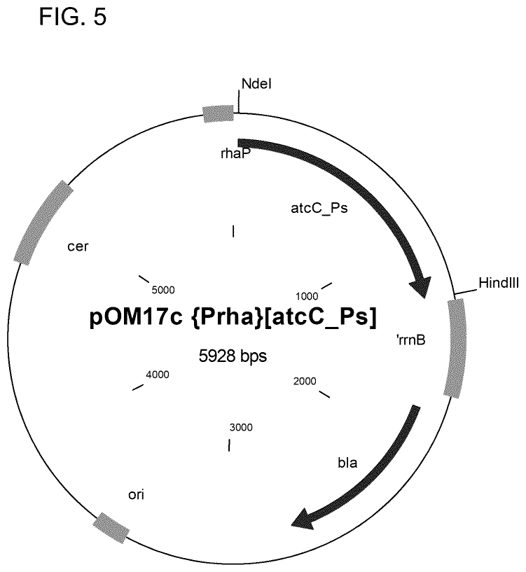

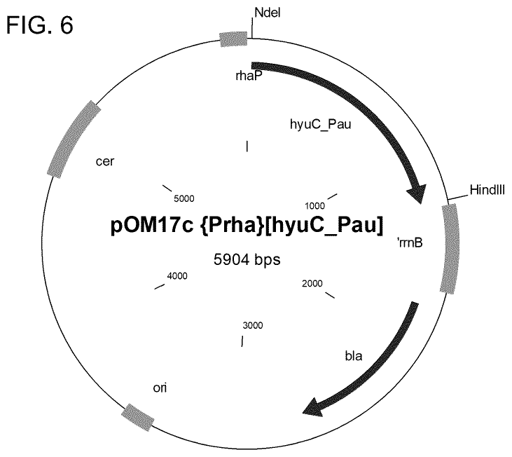

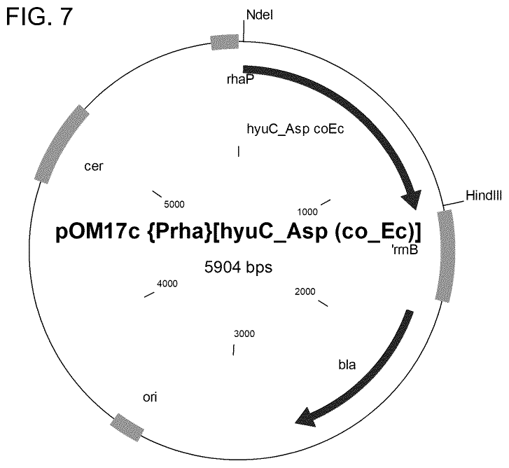

Cloning of the respective L-carbamoylase gene into the rhamnose expression vector pJOE4036 was carried out in a plasmid derivative of the rhamnose expression vector pJOE4036. Polynucleotides comprising the genes of the repective enzymes were synthesized by GeneArt (ThermoFisher Scientific (Waltham, USA)). The polynucleotides carried additional sequences for ndel and HindIII restriction sites. Each one of the genes encoding the enzymes was cloned into pJOE4036 using those restriction sites resulting in the respective plasmid, i.e. pOM17c for expression of SEQ ID NO: 1 (shown in FIG. 3), pOM17c{Prha}[amaB_Gst] for expression of SEQ ID NO: 2 (shown in FIG. 4), pOM17c{Prha}[atcC_Ps] for expression of SEQ ID NO: 3 (shown in FIG. 5), pOM17c{Prha}[hyuc_Pau] for expression of SEQ ID NO: 5 (shown in FIG. 6), pOM17c {Prha}[hyuC_Asp (co_Ec)] for expression of SEQ ID NO: 8 (shown in FIG. 7), each under the control of a rhamnose promotor.

### 5.6 Example 4: Production of Strains Positive for L-Carbamoylase

Chemically competent E. coli ET5 cells (as described in WO 2004/042047 A1) were transformed with 10 ng of the respective plasmid generated according to Example 3.

An E. coli ET5 strain transformed with the respective plasmid was incubated under shaking (250 U/min) at 30° C. for 18 hours in LB medium containing ampicillin (100 μg/l), and 2 g/l rhamnose.

The biomass was separated by centrifugation, resuspended in 50 mM phosphate buffer (pH 7.2) and applied in biotransformation tests in the following examples. The concentration of the biomass in the solution was 12.2 g/l. The solution was used as catalyst (“catalyst 2”) in the following.

The concentration of the respective polypeptide carbamoylase in the obtained solution may be determined by SDS page and analysis of the respective bands via the software GelQuant® (BiochemLabSolutions).

In the following examples, the different carbamolyases were tested to determine whether the respective polpypetide catalyzed the reaction of the carbamoyl substrate enantioselectively to give the corresponding L amino acid.

### 5.6.1 Inventive Example 13

In inventive example 13, a racemic mixture M_{I3} of 0.25 mmol L-(VII)_{I3} and 0.25 mmol D-(VII)_{I3}, wherein L-(VII)_{I3} and D-(VII)_{I3} were carbamoyles with the following formulae, was used as substrate:

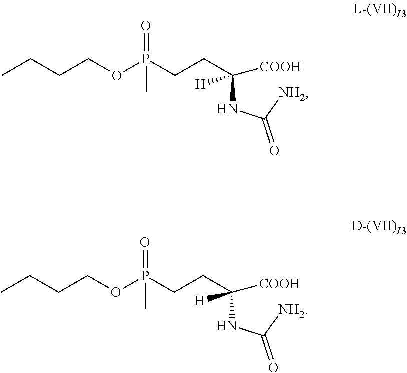 [SMILES: CCCCOP(C)(=O)CC[C@H](NC(N)=O)C(=O)O ; CCCCOP(C)(=O)CC[C@@H](NC(N)=O)C(=O)O]

The mixture M_{I3} was dissolved in a stirring reactor with 25 ml water. 2.4 g of the catalyst 2 was added, followed by the addition of CoCl₂ Solution. The suspension was set to pH 7.4 with 0.5 M NaOH. The total volume was replenished with water to 50 ml, so that the concentration of each compound L-(VII)_{I3} and D-(VII)_{I3} was 5 mmol/l and the concentration of CoCl₂ in the final solution was 1 mM. The pH was held between 7.4 by HCl-titration or NaOH-titration. The temperature was maintained at 37° C. by a thermostat during the reaction.

The reaction was stopped after 48 hours by addition of 2 N HCl until pH 2.5 was reached. The biomass was separated by centrifugation or filtration.

The enzyme reaction was monitored by ninhydrin test to determine the formation of amino acids.

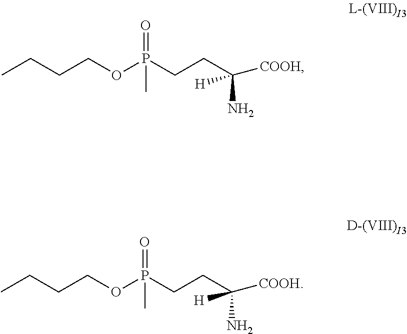

The overall conversion of L-(VII)_{I3} to the respective product L-(VIII)_{I3} and of D-(VII)_{I3} to the respective product D-(VIII)_{I3} after 48 hours was quantified and is shown in table 6. For this analysis, the column: Chiralpak 3 μm ZWIX (+), 150×3 mm, DAICEL was used.

Table 6 gives the yield of L-(VIII)_{I3} relative to the compound L-(VII)_{I3} employed and the yield of D-(VIII)_{I3} relative to the compound D-(VII)_{I3} employed.

TABLE 6

| | | | Conversion of | Conversion of |
|---|---|---|---|---|
| | | | L-(VII)_{I3} → | D-(VII)_{I3} → |
| | Gene | SEQ | L-(VIII)_{I3}, | D-(VIII)_{I3}, |
| Donor Strain | name | ID NO: | in % | in % |
| Arthrobacter | hyuC | SEQ ID | 86.4 | n.d. |
| sp. DSM 3747 | | NO: 1 | | |
| Geobacillus | amaB | SEQ ID | 61.8 | n.d. |
| stearothermophilus | | NO: 2 | | |
| Pseudomonas | atcC | SEQ ID | 42.6 | n.d. |
| sp. QR-101 | | NO: 3 | | |
| Paenarthrobacter | hyuC | SEQ ID | 77.6 | n.d. |
| aurescens | | NO: 5 | | |
| Arthrobacter | hyuC | SEQ ID | 36.2 | n.d. |
| sp. BT801 | | NO: 8 | | |

n.d. = “not detectable”.

### 5.6.2 Conclusion 2

The results as summarized in table 6 are further evidence that certain carbamoylate esters such as the alkylated carbamoylate esters can be used as substrates for the enzymatic enantioselective synthesis of L-glufosinate. It further shows that these carbamoylases specifically catalyse the conversion of the L-glufosinate carbamoylate, but not the D-glufosinate carbamoylate.

1. A method for the production of an L-glufosinate P-alkyl ester according to formula L-(I), the method comprising:
reacting a compound according to formula L-(II) to give a compound according to formula L-(I): 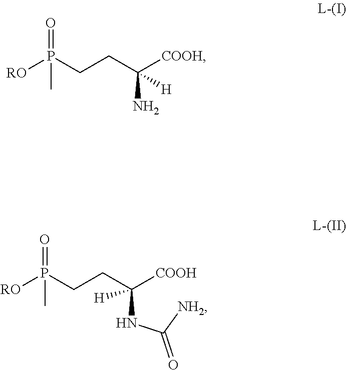
wherein the reaction of L-(II) to give L-(I) is catalyzed by a carbamoylase E₁,
and wherein R is an alkyl group or an aryl group.

2. The method according to claim 1, wherein R is an alkyl group.

3. The method according to claim 1, wherein the reaction of L-(II) to give L-(I) is catalyzed by an L-carbamoylase E₁.

4. The method according to claim 3, wherein the L-carbamoylase E₁ is categorized in the EC class 3.5.1.87.

5. The method according to claim 3, wherein a polypeptide sequence of the L-carbamoylase E₁ is selected from the group consisting of SEQ ID NO: 1 and variants thereof, SEQ ID NO: 2 and variants thereof, SEQ ID NO: 3 and variants thereof, SEQ ID NO: 4 and variants thereof, SEQ ID NO: 5 and variants thereof, SEQ ID NO: 6 and variants thereof, SEQ ID NO: 7 and variants thereof, SEQ ID NO: 8 and variants thereof, and SEQ ID NO: 9 and variants thereof.

6. The method according to claim 1, wherein the compound according to formula L-(II) is obtained by reacting a compound according to formula L-(III) to give a compound according to formula L-(II): 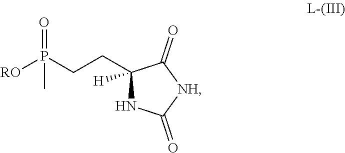
wherein the reaction of L-(III) to give L-(II) is catalyzed by a hydantoinase E₂,
and wherein R has the same meaning as described for L-(I).

7. The method according to claim 6, wherein the hydantoinase E_{Z} is categorized in the EC class 3.5.2.2.

8. The method according to claim 6, wherein a polypeptide sequence of the hydantoinase E₂ is selected from the group consisting of SEQ ID NO: 10 and variants thereof, SEQ ID NO: 11 and variants thereof, SEQ ID NO: 12 and variants thereof, SEQ ID NO: 13 and variants thereof, SEQ ID NO: 14 and variants thereof, SEQ ID NO: 15 and variants thereof, and SEQ ID NO: 16 and variants thereof.

9. The method according to claim 6, wherein the hydantoinase E_{Z} is an L-hydantoinase E₂.

10. The method according to claim 6, wherein the compound according to formula L-(III) is obtained by reacting a compound according to formula D-(III) to give a compound according to formula L-(III): 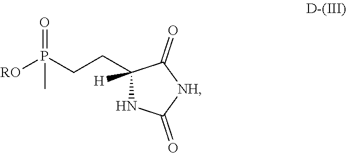
and wherein R has the same meaning as described for L-(I).

11. The method according to claim 10, wherein the reaction of D-(III) to give L-(III) is catalyzed by a hydantoin racemase E₃.

12. The method according to claim 11, wherein the hydantoin racemase E₃ is categorized in the EC class 5.1.99.5.

13. The method according to claim 11, wherein a the polypeptide sequence of the hydantoin racemase E₃ is selected from the group consisting of SEQ ID NO: 17 and variants thereof, SEQ ID NO: 18 and variants thereof, SEQ ID NO: 19 and variants thereof, SEQ ID NO: 20 and variants thereof, SEQ ID NO: 21 and variants thereof, SEQ ID NO: 22 and variants thereof, SEQ ID NO: 23 and variants thereof, SEQ ID NO: 24 and variants thereof, SEQ ID NO: 25 and variants thereof, and SEQ ID NO: 26 and variants thereof.

14. A method for the production of an L-glufosinate P-alkyl ester according to formula L-(I), wherein R is an alkyl group or aryl group: 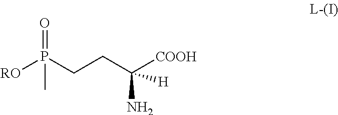
the method comprising:
(i-A) providing a mixture M_{IIIA} comprising both enantiomers L-(III) and D-(III),
wherein R has the same meaning as described for L-(I): 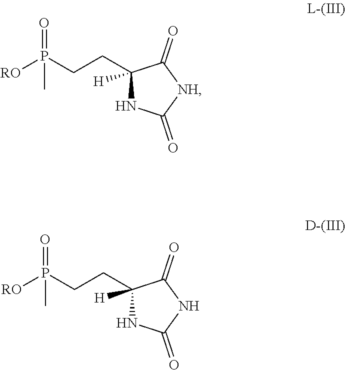
(i-B) optionally reacting at least a part of the compounds D-(III) comprised by the mixture M_{IIIA} into L-(III), giving a composition M_{IIIB} comprising L-(III) and optionally its enantiomer D-(III);
(ii) subjecting mixture M_{IIIA} or, in case (i-B) is carried out, composition M_{IIIB}, to a reaction where a compound according to formula L-(III) is reacted to give a compound according to formula L-(II), giving a composition M_{II} comprising L-(II) and optionally its enantiomer D-(II), wherein L-(II) and D-(II) have the following formulae and wherein R in formulae L-(II) and D-(II) has the same meaning as described for L-(I): 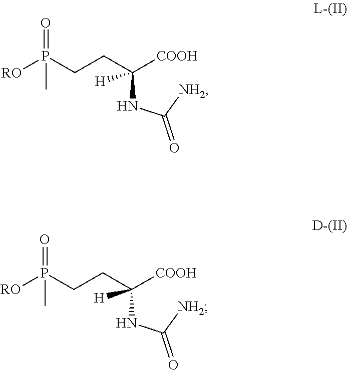
(iii) subjecting M_{II} to a reaction where a compound according to formula L-(II) is reacted to give a compound according to formula L-(I), giving a composition M_{I} comprising L-(I) and optionally its enantiomer D-(I), wherein, in case M_{I} comprises both enantiomers L-(I) and D-(I), a molar ratio of L-(I) to D-(I) in M_{I} is greater than a molar ratio of L-(III) to D-(III) in M_{IIIA}, wherein D-(I) has the following formula and wherein R in formula D-(I) has the same meaning as described for L-(I):

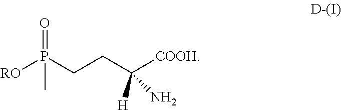

15. The method according to claim 1, wherein the compound according to formula L-(I) is saponified to give L-glufosinate.

16. The method according to claim 14, wherein the compound according to formula L-(I) is saponified to give L-glufosinate.
